(19) United States
(12) Patent Application Publication
(10) Pub. No.: US 2025/0040934 A1
(43) Pub. Date: Feb. 6, 2025

(54) Janjua Toroidal (JAN-T) Intrasaccular Aneurysm Occlusion Device

(71) Applicant: Robert A. Connor, Wyoming, MN (US)

(72) Inventor: Robert A. Connor, Wyoming, MN (US)

(73) Assignee: Aneuclose LLC, Ham Lake, MN (US)

(21) Appl. No.: 18/920,939

(22) Filed: Oct. 20, 2024

Related U.S. Application Data
(63) Continuation-in-part of application No. 18/760,322, filed on Jul. 1, 2024.
(63) Continuation-in-part of application No. 18/674,996, filed on May 27, 2024.
Application No. 18/760,322 is a continuation-in-part of application No. 18/674,996, filed on May 27, 2024.
Application No. 18/674,996 is a continuation-in-part of application No. 18/613,053, filed on Mar. 21, 2024, now abandoned.
Application No. 18/613,053 is a continuation-in-part of application No. 17/970,510, filed on Oct. 20, 2022, now abandoned.
Application No. 18/674,996 is a continuation-in-part of application No. 18/613,053, filed on Mar. 21, 2024, now abandoned.
Application No. 18/613,053 is a continuation-in-part of application No. 18/519,055, filed on Nov. 26, 2023, now Pat. No. 12,004,750.
Application No. 18/613,053 is a continuation-in-part of application No. 18/519,055, filed on Nov. 26, 2023, now Pat. No. 12,004,750.
Application No. 18/519,055 is a continuation-in-part of application No. 18/135,153, filed on Apr. 15, 2023, now abandoned.
Application No. 18/519,055 is a continuation-in-part of application No. 18/374,602, filed on Sep. 28, 2023, now Pat. No. 11,944,316.
Application No. 18/374,602 is a continuation-in-part of application No. 18/135,153, filed on Apr. 15, 2023, now abandoned.
Application No. 18/135,153 is a continuation-in-part of application No. 17/970,510, filed on Oct. 20, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 18/374,602 is a continuation-in-part of application No. 18/135,153, filed on Apr. 15, 2023, now abandoned.
Application No. 18/135,153 is a continuation-in-part of application No. 17/970,510, filed on Oct. 20, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 18/135,153 is a continuation-in-part of application No. 17/970,510, filed on Oct. 20, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 17/829,313 is a continuation-in-part of application No. 17/476,845, filed on Sep. 16, 2021, now Pat. No. 11,583,289.
Application No. 17/965,502 is a continuation-in-part of application No. 17/829,313, filed on May 31, 2022, now abandoned.
Application No. 17/829,313 is a continuation-in-part of application No. 17/476,845, filed on Sep. 16, 2021, now Pat. No. 11,583,289.
Application No. 17/829,313 is a continuation-in-part of application No. 17/485,390, filed on Sep. 25, 2021, now Pat. No. 11,471,164.
Application No. 17/485,390 is a continuation-in-part of application No. 17/476,845, filed on Sep. 16, 2021, now Pat. No. 11,583,289.
Application No. 17/476,845 is a continuation-in-part of application No. 17/472,674, filed on Sep. 12, 2021, now abandoned.
Application No. 17/472,674 is a continuation-in-part of application No. 17/467,680, filed on Sep. 7, 2021, now abandoned.
Application No. 17/467,680 is a continuation-in-part of application No. 17/466,497, filed on Sep. 3, 2021, now Pat. No. 11,357,511.
Application No. 17/466,497 is a continuation-in-part of application No. 17/353,652, filed on Jun. 21, 2021, now abandoned.
Application No. 17/353,652 is a continuation-in-part of application No. 17/220,002, filed on Apr. 1, 2021, now Pat. No. 11,464,518.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/211,446 is a continuation-in-part of application No. 16/693,267, filed on Nov. 23, 2019, now abandoned.
Application No. 16/693,267 is a continuation-in-part of application No. 16/660,929, filed on Oct. 23, 2019, now Pat. No. 11,471,163.
Application No. 16/693,267 is a continuation-in-part of application No. 16/660,929, filed on Oct. 23, 2019, now Pat. No. 11,471,163.
Application No. 16/660,929 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 15/865,822, filed on Jan. 9, 2018, now Pat. No. 10,716,573.
Application No. 15/865,822 is a continuation-in-part of application No. 15/081,909, filed on Mar. 27, 2016, now abandoned.
Application No. 15/081,909 is a continuation-in-part of application No. 14/526,600, filed on Oct. 29, 2014, now abandoned.
Application No. 14/526,600 is a continuation-in-part of application No. 15/080,915, filed on Mar. 25, 2016, now Pat. No. 10,028,747.
Application No. 15/080,915 is a continuation-in-part of application No. 14/526,600, filed on Oct. 29, 2014, now abandoned.
Application No. 15/081,909 is a continuation-in-part of application No. 14/526,600, filed on Oct. 29, 2014, now abandoned.
Application No. 15/080,915 is a continuation-in-part of application No. 14/526,600, filed on Oct. 29, 2014, now abandoned.
Application No. 14/526,600 is a continuation-in-part of application No. 12/989,048, filed on Oct. 21, 2010, now Pat. No. 8,974,487.
(60) Provisional application No. 63/119,774, filed on Dec. 1, 2020.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/720,173, filed on Aug. 21, 2018.
(60) Provisional application No. 62/589,754, filed on Nov. 22, 2017.
(60) Provisional application No. 62/472,519, filed on Mar. 16, 2017.
(60) Provisional application No. 62/589,754, filed on Nov. 22, 2017.
(60) Provisional application No. 62/472,519, filed on Mar. 16, 2017.
(60) Provisional application No. 62/444,860, filed on Jan. 11, 2017.
(60) Provisional application No. 61/897,245, filed on Oct. 30, 2013.
(60) Provisional application No. 61/126,047, filed on May 1, 2008.
(60) Provisional application No. 61/126,027, filed on May 1, 2008.

(51) Int. Cl. A61B 17/12

(52) U.S. Cl. CPC A61B 17/12113; A61B 17/12031; A61B 17/1214; A61B 17/12181; A61B 2017/1205

(57) ABSTRACT

This invention is a torus (or torus-section) shaped intrasaccular aneurysm occlusion device. A torus (or torus section) shape can be modeled by revolving a convex shape around an axis of revolution which is coplanar with the convex shape. The convex shape which is revolved can be a circle, an ellipse, or half of a yin-yang symbol. The device can further comprise one or more embolic members or embolic material which is inserted into the aneurysm sac through a central opening in the neck bridge.

### CROSS-REFERENCE TO RELATED APPLICATIONS

This application is a continuation-in-part of U.S. patent application Ser. No. 18/760,322 filed on 2024 Jul. 1 and a continuation-in-part of U.S. patent application Ser. No. 18/674,996 filed on 2024 May 27. U.S. patent application Ser. No. 18/760,322 was a continuation-in-part of U.S. patent application Ser. No. 18/674,996 filed on 2024 May 27, a continuation-in-part of U.S. patent application Ser. No. 18/613,053 filed on 2024 Mar. 21, and a continuation-in-part of U.S. patent application Ser. No. 17/970,510 filed on 2022 Oct. 20.
U.S. patent application Ser. No. 18/674,996 was a continuation-in-part of U.S. Pat. No. 18,613,053 filed on 2024 Mar. 21 and a continuation-in-part of U.S. patent application Ser. No. 18/519,055 filed on 2023 Nov. 26. U.S. patent application Ser. No. 18/613,053 was a continuation-in-part of U.S. patent application Ser. No. 18/519,055 filed on 2023 Nov. 26 and a continuation-in-part of U.S. patent application Ser. No. 18/135,153 filed on 2023 Apr. 15.
U.S. patent application Ser. No. 18/519,055 was a continuation-in-part of U.S. patent application Ser. No. 18/374,602 filed on 2023 Sep. 28, a continuation-in-part of U.S. patent application Ser. No. 18/135,153 filed on 2023 Apr. 15, a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31.
U.S. patent application Ser. No. 18/374,602 was a continuation-in-part of U.S. patent application Ser. No. 18/135,153 filed on 2023 Apr. 15, a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31.
U.S. patent application Ser. No. 18/135,153 was a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31. U.S. patent application Ser. No. 17/970,510 was a continuation-in-part of U.S. patent application Ser. No. 17/965,502 filed on 2022 Oct. 13, a continuation-in-part of U.S. patent application Ser. No. 17/829,313 filed on 2022 May 31, and a continuation-in-part of U.S. patent application Ser. No. 17/476,845 filed on 2021 Sep. 16.
U.S. patent application Ser. No. 17/829,313 was a continuation-in-part of U.S. patent application Ser. No. 17/485,390 filed on 2021 Sep. 25, was a continuation-in-part of U.S. patent application Ser. No. 17/476,845 filed on 2021 Sep. 16, was a continuation-in-part of U.S. patent application Ser. No. 17/472,674 filed on 2021 Sep. 12, was a continuation-in-part of U.S. patent application Ser. No. 17/467,680 filed on 2021 Sep. 7, was a continuation-in-part of U.S. patent application Ser. No. 17/466,497 filed on 2021 Sep. 3, was a continuation-in-part of U.S. patent application Ser. No. 17/353,652 filed on 2021 Jun. 21, was a continuation-in-part of U.S. patent application Ser. No. 17/220,002 filed on 2021 Apr. 1, was a continuation-in-part of U.S. patent application Ser. No. 17/214,827 filed on 2021 Mar. 27, was a continuation-in-part of U.S. patent application Ser. No. 17/211,446 filed on 2021 Mar. 24, was a continuation-in-part of U.S. patent application Ser. No. 16/693,267 filed on 2019 Nov. 23, and was a continuation-in-part of U.S. patent application Ser. No. 16/660,929 filed on 2019 Oct. 23.
U.S. patent application Ser. No. 17/220,002 was a continuation-in-part of U.S. patent application Ser. No. 17/214,827 filed on 2021 Mar. 27. U.S. patent application Ser. No. 17/220,002 was a continuation-in-part of U.S. patent application Ser. No. 17/211,446 filed on 2021 Mar. 24. U.S. patent application Ser. No. 17/220,002 claimed the priority benefit of U.S. provisional patent application 63/119,774 filed on 2020 Dec. 1. U.S. patent application Ser. No. 17/220,002 was a continuation-in-part of U.S. patent application Ser. No. 16/693,267 filed on 2019 Nov. 23. U.S. patent application Ser. No. 17/220,002 was a continuation-in-part of U.S. patent application Ser. No. 16/660,929 filed on 2019 Oct. 23.
U.S. patent application Ser. No. 16/693,267 was a continuation-in-part of U.S. patent application Ser. No. 16/660,929 filed on 2019 Oct. 23. U.S. patent application Ser. No. 16/693,267 claimed the priority benefit of U.S. provisional patent application 62/794,609 filed on 2019 Jan. 19. U.S. patent application Ser. No. 16/693,267 claimed the priority benefit of U.S. provisional patent application 62/794,607 filed on 2019 Jan. 19. U.S. patent application Ser. No. 16/693,267 was a continuation-in-part of U.S. patent application Ser. No. 16/541,241 filed on 2019 Aug. 15. U.S. patent application Ser. No. 16/693,267 was a continuation-in-part of U.S. patent application Ser. No. 15/865,822 filed on 2018 Jan. 9 which issued as U.S. Pat. No. 10,716,573 on 2020 Jul. 21. U.S. patent application Ser. No. 16/693,267 was a continuation-in-part of U.S. patent application Ser. No. 15/861,482 filed on 2018 Jan. 3.
U.S. patent application Ser. No. 16/660,929 claimed the priority benefit of U.S. provisional patent application 62/794,609 filed on 2019 Jan. 19. U.S. patent application Ser. No. 16/660,929 claimed the priority benefit of U.S. provisional patent application 62/794,607 filed on 2019 Jan. 19. U.S. patent application Ser. No. 16/660,929 was a continuation-in-part of U.S. patent application Ser. No. 16/541,241 filed on 2019 Aug. 15. U.S. patent application Ser. No. 16/660,929 was a continuation-in-part of U.S. patent application Ser. No. 15/865,822 filed on 2018 Jan. 9 which issued as U.S. Pat. No. 10,716,573 on 2020 Jul. 21. U.S. patent application Ser. No. 16/660,929 was a continuation-in-part of U.S. patent application Ser. No. 15/861,482 filed on 2018 Jan. 3.
U.S. patent application Ser. No. 16/541,241 claimed the priority benefit of U.S. provisional patent application 62/794,609 filed on 2019 Jan. 19. U.S. patent application Ser. No. 16/541,241 claimed the priority benefit of U.S. provisional patent application 62/794,607 filed on 2019 Jan. 19. U.S. patent application Ser. No. 16/541,241 claimed the priority benefit of U.S. provisional patent application 62/720,173 filed on 2018 Aug. 21. U.S. patent application Ser. No. 16/541,241 was a continuation-in-part of U.S. patent application Ser. No. 15/865,822 filed on 2018 Jan. 9 which issued as U.S. Pat. No. 10,716,573 on 2020 Jul. 21
U.S. patent application Ser. No. 15/865,822 claimed the priority benefit of U.S. provisional patent application 62/589,754 filed on 2017 Nov. 22. U.S. patent application Ser. No. 15/865,822 claimed the priority benefit of U.S. provisional patent application 62/472,519 filed on 2017 Mar. 16. U.S. patent application Ser. No. 15/865,822 was a continuation-in-part of U.S. patent application Ser. No. 15/081,909 filed on 2016 Mar. 27. U.S. patent application Ser. No. 15/865,822 was a continuation-in-part of U.S. patent application Ser. No. 14/526,600 filed on 2014 Oct. 29.
U.S. patent application Ser. No. 15/861,482 claimed the priority benefit of U.S. provisional patent application 62/589,754 filed on 2017 Nov. 22. U.S. patent application Ser. No. 15/861,482 claimed the priority benefit of U.S. provisional patent application 62/472,519 filed on 2017 Mar. 16. U.S. patent application Ser. No. 15/861,482 claimed the priority benefit of U.S. provisional patent application 62/444,860 filed on 2017 Jan. 11. U.S. patent application Ser. No. 15/861,482 was a continuation-in-part of U.S. patent application Ser. No. 15/080,915 filed on 2016 Mar. 25 which issued as U.S. Pat. No. 10,028,747 on 2018 Jul. 24. U.S. patent application Ser. No. 15/861,482 was a continuation-in-part of U.S. patent application Ser. No. 14/526,600 filed on 2014 Oct. 29.
U.S. patent application Ser. No. 15/081,909 was a continuation-in-part of U.S. patent application Ser. No. 14/526,600 filed on 2014 Oct. 29. U.S. patent application Ser. No. 15/080,915 was a continuation-in-part of U.S. patent application Ser. No. 14/526,600 filed on 2014 Oct. 29. U.S. patent application Ser. No. 14/526,600 claimed the priority benefit of U.S. provisional patent application 61/897,245 filed on 2013 Oct. 30. U.S. patent application Ser. No. 14/526,600 was a continuation-in-part of U.S. patent application Ser. No. 12/989,048 filed on 2010 Oct. 21 which issued as U.S. Pat. No. 8,974,487 on 2015 Mar. 10. U.S. patent application Ser. No. 12/989,048 claimed the priority benefit of U.S. provisional patent application 61/126,047 filed on 2008 May 1. U.S. patent application Ser. No. 12/989,048 claimed the priority benefit of U.S. provisional patent application 61/126,027 filed on 2008 May 1.

The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to aneurysm occlusion devices and methods.

### Introduction

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain. Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Even among those who survive, approximately one-half suffer significant and permanent deterioration of brain function. Better alternatives for cerebral aneurysm treatment are needed.

### Review of the Relevant Art

U.S. patent application No. 20020169473 (Sepetka et al., Nov. 14, 2002, “Devices and Methods for Treating Vascular Malformations”) discloses a device with a primary coil to provide structural integrity and secondary windings to fill interstitial spaces. U.S. patent applications 20060155323 (Porter et al., Jul. 13, 2006, “Intra-Aneurysm Devices”) and 20190298379 (Porter et al., Oct. 3, 2019, “Intra-Aneurysm Devices”) and also U.S. Pat. No. 10,265,075 (Porter et al., Apr. 23, 2019, “Intra-Aneurysm Devices”) disclose an occlusive device having a neck and a dome. U.S. patent application No. 20080281350 (Sepetka et al., Nov. 13, 2008, “Aneurysm Occlusion Devices”) discloses an implantable occlusion device with a concave or cup-shaped shape after implantation.

U.S. patent application No. 20110022149 (Cox et al., Jan. 27, 2011, “Methods and Devices for Treatment of Vascular Defects”) discloses a device with first ends secured to a first ring and second ends secured to a second ring with the first and second rings being disposed substantially concentric to the longitudinal axis. U.S. patent application No. 20110208227 (Becking, Aug. 25, 2011, “Filamentary Devices for Treatment of Vascular Defects”) discloses braid-balls for aneurysm occlusion. U.S. patent application No. 20120165919 (Cox et al., Jun. 28, 2012, “Methods and Devices for Treatment of Vascular Defects”) and U.S. patent application No. 20140052233 (Cox et al., Feb. 20, 2014, “Methods and Devices for Treatment of Vascular Defects”) disclose an expandable wire body support structure having a low profile radially constrained state, an expanded relaxed state with a substantially spherical or globular configuration having a smooth outer surface, and a porous permeable layer comprising a braided wire occlusive mesh.

U.S. patent application No. 20120283768 (Cox et al., Nov. 8, 2012, “Method and Apparatus for the Treatment of Large and Giant Vascular Defects”) discloses the deployment of multiple permeable shell devices within a single vascular defect. U.S. patent application No. 20120316598 (Becking et al., Dec. 13, 2012, “Multiple Layer Filamentary Devices for Treatment of Vascular Defects”) and U.S. Pat. No. 9,585,669 (Becking et al., Mar. 17, 2017, “Multiple Layer Filamentary Devices for Treatment of Vascular Defects”) disclose braid balls for aneurysm occlusion. U.S. patent application 20130245667 (Marchand et al., Sep. 19, 2013, “Filamentary Devices and Treatment of Vascular Defects”) discloses a self-expanding resilient permeable shell wherein filaments are bundled and secured to each other at a proximal end.

U.S. Pat. No. 8,597,320 (Sepetka et al., Dec. 3, 2013, “Devices and Methods for Treating Vascular Malformations”) discloses a method of filling an aneurysm by advancing a device with a proximal collar and a distal collar through a vascular system and then positioning the device within an aneurysm. U.S. patent applications 20140135811 (Divino et al., May 15, 2014, “Occlusive Devices”), 20140135812 (Divino et al., May 15, 2014, “Occlusive Devices”), 20190282242 (Divino et al., Sep. 19, 2019, “Occlusive Devices”), and 20190290286 (Divino et al., Sep. 26, 2019, “Occlusive Devices”) and also U.S. Pat. No. 10,327,781 (Divino et al., Jun. 25, 2019, “Occlusive Devices”) disclose multiple expandable structures, wherein each of the expandable structures has a unique shape or porosity profile.

U.S. patent application No. 20140200607 (Sepetka et al., Jul. 17, 2014, “Occlusive Device”), U.S. patent application No. 20190274691 (Sepetka et al., Sep. 12, 2019, “Occlusive Device”), and U.S. Pat. No. 11,045,203 (Sepetka et al., Jun. 29, 2021, “Occlusive Device”) disclose multiple sequentially deployed occlusive devices that are connected together to create an extended length. U.S. patent application 20140330299 (Rosenbluth et al., Nov. 6, 2014, “Embolic Occlusion Device and Method”), U.S. patent application No. 20180303486 (Rosenbluth et al., Oct. 25, 2018, “Embolic Occlusion Device and Method”), and U.S. patent application No. 20210259699 (Rosenbluth et al., Aug. 26, 2021, “Embolic Occlusion Device and Method”) disclose an occlusion device with a tubular braided member having a first end and a second end and extending along a longitudinal axis, the tubular braided member having a repeating pattern of larger diameter portions and smaller diameter portions arrayed along the longitudinal axis.

U.S. patent application No. 20140358178 (Hewitt et al., Dec. 4, 2014, “Filamentary Devices for Treatment of Vascular Defects”), U.S. Pat. No. 9,078,658 (Hewitt et al., Jul. 14, 2015, “Filamentary Devices for Treatment of Vascular Defects”), U.S. patent application No. 20160249934 (Hewitt et al., Sep. 1, 2016, “Filamentary Devices for Treatment of Vascular Defects”), U.S. Pat. No. 9,955,976 (Hewitt et al., May 1, 2018, “Filamentary Devices for Treatment of Vascular Defects”), U.S. patent application 20180206849 (Hewitt et al., Jul. 26, 2018, “Filamentary Devices for the Treatment of Vascular Defects”), U.S. patent application No. 20210007754 (Milhous et al., Jan. 14, 2021, “Filamentary Devices for Treatment of Vascular Defects”), U.S. Pat. No. 10,939,914 (Hewitt et al., Mar. 9, 2021, “Filamentary Devices for the Treatment of Vascular Defects”), and U.S. patent application No. 20210275184 (Hewitt et al., Sep. 9, 2021, “Filamentary Devices for Treatment of Vascular Defects”) disclose occlusion devices with permeable shells made of woven braided mesh having a variable mesh density and/or porosity.

U.S. Pat. No. 8,998,947 (Aboytes et al., Apr. 7, 2015, “Devices and Methods for the Treatment of Vascular Defects”) discloses an expandable implant that with a first configuration in which the first portion and the second portion are substantially linearly aligned and a second configuration in which the second portion at least partially overlaps the first portion. U.S. Pat. No. 9,039,726 (Becking. May 26, 2015, “Filamentary Devices for Treatment of Vascular Defects”) discloses braid-balls for aneurysm occlusion. U.S. patent applications 20150272589 (Lorenzo, Oct. 1, 2015, “Aneurysm Occlusion Device”) and 20190008522 (Lorenzo, Jan. 10, 2019, “Aneurysm Occlusion Device”) and also U.S. Pat. No. 11,076,860 (Lorenzo, Aug. 3, 2021, “Aneurysm Occlusion Device”) disclose a tubular structure which is constrained by a control ring.

U.S. patent applications 20150313605 (Griffin, Nov. 5, 2015, “Occlusion Device”) and 20190059909 (Griffin, Feb. 28, 2019, “Occlusion Device”) and also U.S. Pat. No. 10,130,372 (Griffin, Nov. 20, 2018, “Occlusion Device”) and U.S. Pat. No. 11,389,174 (Griffin, Jul. 19, 2022, “Occlusion Device”) disclose an occlusion device with a substantially solid marker having a proximal end, and a distal end; and a low profile resilient mesh body attached to the distal end of the marker. U.S. patent applications 20160022445 (Ruvalcaba et al., Jan. 28, 2016, “Occlusive Device”) and 20190343664 (Ruvalcaba et al., Nov. 14, 2019, “Occlusive Device”) U.S. Pat. No. 11,389,309 (Ruvalcaba et al., Jul. 19, 2022, “Occlusive Device”) disclose an aneurysm embolization device having a single, continuous piece of material that is shape set into a plurality of distinct structural components.

U.S. patent application No. 20160249935 (Hewitt et al., Sep. 1, 2016, “Devices for Therapeutic Vascular Procedures”), U.S. patent application No. 20160367260 (Hewitt et al., Dec. 22, 2016, “Devices for Therapeutic Vascular Procedures”), U.S. Pat. No. 9,629,635 (Hewitt et al., Apr. 25, 2017, “Devices for Therapeutic Vascular Procedures”), and U.S. patent application No. 20170128077 (Hewitt et al., May 11, 2017. “Devices for Therapeutic Vascular Procedures”) disclose a self-expanding resilient permeable shell and a metallic coil secured to the distal end of the permeable shell. U.S. patent application No. 20160249937 (Marchand et al., Sep. 1, 2016, “Multiple Layer Filamentary Devices for Treatment of Vascular Defects”), U.S. Pat. No. 9,918,720 (Marchand et al., Mar. 20, 2018, “Multiple Layer Filamentary Devices for Treatment of Vascular Defects”), and U.S. Pat. No. 10,238,393 (Marchand et al., Mar. 26, 2019, “Multiple Layer Filamentary Devices for Treatment of Vascular Defects”) disclose a permeable shell and an inner structure configured to occlude blood flow.

U.S. Pat. No. 9,492,174 (Hewitt et al., Nov. 15, 2016, “Filamentary Devices for Treatment of Vascular Defects”), U.S. patent application No. 20170095254 (Hewitt et al., Apr. 6, 2017, “Filamentary Devices for Treatment of Vascular Defects”), U.S. Pat. No. 10,136,896 (Hewitt et al., Nov. 27, 2018, “Filamentary Devices for Treatment of Vascular Defects”), U.S. patent application No. 20190192166 (Hewitt et al., Jun. 27, 2019, “Filamentary Devices for Treatment of Vascular Defects”), U.S. patent application No. 20200289124 (Rangwala et al., Sep. 17, 2020, “Filamentary Devices for Treatment of Vascular Defects”), U.S. Pat. No. 10,813,645 (Hewitt et al., Oct. 27, 2020, “Filamentary Devices for Treatment of Vascular Defects”), and U.S. patent application No. 20210106337 (Hewitt et al., Apr. 15, 2021, “Filamentary Devices for Treatment of Vascular Defects”) disclose a self-expanding permeable shell having a radially constrained elongated state configured for delivery within a catheter lumen, an expanded state with a globular and longitudinally shortened configuration relative to the radially constrained state, and a plurality of elongate filaments that are woven together.

U.S. patent application No. 20170079661 (Bardsley et al., Mar. 23, 2017, “Occlusive Devices”), U.S. Pat. No. 10,314,593 (Bardsley et al., Jun. 11, 2019, “Occlusive Devices”), and U.S. patent application 20190269411 (Bardsley et al., Sep. 5, 2019, “Occlusive Devices”) disclose an implant with a single- or dual-layer braided body with variable porosity. U.S. patent application No. 20170079662 (Rhee et al., Mar. 23, 2017. “Occlusive Devices”) discloses an implant with a frame and a mesh component, wherein the mesh component has a first porosity and the frame has a second porosity. U.S. patent application No. 20170156734 (Griffin, Jun. 8, 2017, “Occlusion Device”), U.S. Pat. No. 10,285,711 (Griffin, May 14, 2019, “Occlusion Device”), U.S. patent application No. 20190269414 (Griffin, Sep. 5, 2019, “Occlusion Device”), U.S. patent application No. 20210153871 (Griffin, May 27, 2021, “Occlusion Device”), and U.S. patent application No. 20220313274 (Griffin, Oct. 6, 2022, “Occlusion Device”) disclose a continuous compressible mesh structure comprising axial mesh carriages configured end to end, wherein each end of each carriage is a pinch point in the continuous mesh structure.

U.S. patent application No. 20170224350 (Shimizu et al., Aug. 10, 2017, “Devices for Vascular Occlusion”), U.S. Pat. No. 10,729,447 (Shimizu et al., Aug. 4, 2020, “Devices for Vascular Occlusion”), U.S. patent application No. 20200323534 (Shimizu et al., Oct. 15, 2020, “Devices for Vascular Occlusion”). U.S. Pat. No. 10,980,545 (Bowman et al., Apr. 20, 2021, “Devices for Vascular Occlusion”), U.S. patent application No. 20210228214 (Bowman et al., Jul. 29, 2021, “Devices for Vascular Occlusion”), and U.S. patent application No. 20210228214 (Bowman et al., Jul. 29, 2021, “Devices for Vascular Occlusion”) disclose an occlusive device, an occlusive device delivery system, method of using, method of delivering an occlusive device, and method of making an occlusive device to treat various intravascular conditions. U.S. patent application No. 20170258473 (Plaza et al., Sep. 14, 2017, “Systems and Methods for Delivery of Stents and Stent-Like Devices”) discloses an elongate tubular member having a lumen, an expandable stent, and a delivery device which is placed in a cerebral vessel adjacent to an aneurysm.

U.S. patent application No. 20170281194 (Divino et al., Oct. 5, 2017, “Embolic Medical Devices”) discloses an occlusive device with an elongate member having opposing first and second side edges which extend longitudinally along the member and a member width, wherein this member has a collapsed configuration in which the first and second side edges are curled toward each other about a longitudinal axis of the member. U.S. patent application No. 20180000489 (Marchand et al., Jan. 4, 2018, “Filamentary Devices for Treatment of Vascular Defects”) discloses a self-expanding resilient permeable shell having a plurality of elongate resilient filaments with a woven structure. U.S. patent application No. 20180070955 (Greene et al., Mar. 15, 2018, “Embolic Containment”) discloses a method of treating a neurovascular arteriovenous malformation with liquid embolic and dimethyl sulfoxide.

U.S. patent application No. 20180242979 (Lorenzo, Aug. 30, 2018, “Aneurysm Device and Delivery System”) and U.S. Pat. No. 10,751,066 (Lorenzo, Aug. 25, 2020, “Aneurysm Device and Delivery System”) disclose a self-expanding braided tubular member. U.S. patent application No. 20190053811 (Garza et al., Feb. 21, 2019, “Flow Attenuation Device”) and U.S. Pat. No. 11,071,551 (Garza et al., Jul. 27, 2021, “Flow Attenuation Device”) disclose an embolic device for treating aneurysms with a desired porosity only at discrete sections. U.S. patent application No. 20190192168 (Lorenzo et al., Jun. 27, 2019, “Aneurysm Device and Delivery Method”) and U.S. Pat. No. 10,716,574 (Lorenzo et al., Jul. 21, 2020, “Aneurysm Device and Delivery Method”) discloses a self-expanding braid for treating an aneurysm, including a method for inverting and buckling a proximal segment.

U.S. patent application No. 20190223878 (Lorenzo et al., Jul. 25, 2019, “Aneurysm Device and Delivery System”) and U.S. patent application No. 20200397447 (Lorenzo et al., Dec. 24, 2020, “Aneurysm Device and Delivery System”) discloses an expandable segment which radially expands inside an outer occlusive sack. U.S. patent application No. 20190223881 (Hewitt et al., Jul. 25, 2019, “Devices for Therapeutic Vascular Procedures”) discloses a self-expanding resilient permeable shell made from elongate resilient filaments with a distal region that extends beyond the distal end of the permeable shell. U.S. Pat. No. 10,398,441 (Warner et al., Sep. 3, 2019, “Vascular Occlusion”) discloses a vascular treatment system with a containment device, a pusher, and a stopper ring. U.S. patent application No. 20190343532 (Divino et al., Nov. 14, 2019, “Occlusive Devices”) discloses a device with at least one expandable structure which is adapted to transition from a compressed configuration to an expanded configuration when released into an aneurysm.

U.S. Pat. No. 10,478,194 (Rhee et al., Nov. 19, 2019, “Occlusive Devices”) and U.S. patent application No. 20200038032 (Rhee et al., Feb. 6, 2020, “Occlusive Devices”) disclose an implant with a frame and a mesh component, wherein the mesh component has a first porosity and the frame has a second porosity. U.S. patent application No. 20190365385 (Gorochow et al., Dec. 5, 2019, “Aneurysm Device and Delivery System”) and U.S. Pat. No. 10,939,915 (Gorochow et al., Mar. 9, 2021, “Aneurysm Device and Delivery System”) discloses a braid, wherein translating the braid causes a delivery portion to expand and form a distal sack as well as invert into itself. U.S. patent application 20200029973 (Walzman, Jan. 30, 2020, “Mash Cap for Ameliorating Outpouchings”) discloses an embolic device comprising a control element, a catheter element, a delivery microcatheter hypotube, a detachment element, a mesh disc, a distal opening, and at least one attached extension arm.

U.S. patent application No. 20200038034 (Maguire et al., Feb. 6, 2020, “Vessel Occluder”) discloses a vessel occluder with an expandable mesh portion having a flexible membrane that expands within a cavity of the expandable mesh portion. U.S. Pat. No. 10,610,231 (Marchand et al., Apr. 7, 2020, “Filamentary Devices for Treatment of Vascular Defects”) discloses a self-expanding resilient permeable shell with a plurality of elongate resilient filaments with a woven structure, wherein the plurality of filaments includes small filaments and large filaments, and wherein the small filaments have a transverse dimension which is smaller than the transverse dimension of the large filaments.

U.S. patent application No. 20200113576 (Gorochow et al., Apr. 16, 2020, “Folded Aneurysm Treatment Device and Delivery Method”) and U.S. patent application No. 20210196284 (Gorochow et al., Jul. 1, 2021, “Folded Aneurysm Treatment Device and Delivery Method”) disclose an implant having a braided section that folds to form an outer occlusive sack extending across a neck of an aneurysm to engage a wall of the aneurysm from within a sac of the aneurysm and an inner occlusive sack forming a trough nested within the outer occlusive sack.

U.S. Pat. No. 10,653,425 (Gorochow et al., May 19, 2020, “Layered Braided Aneurysm Treatment Device”), U.S. patent application No. 20200367893 (Xu et al., Nov. 26, 2020, “Layered Braided Aneurysm Treatment Device”), U.S. patent application No. 20200367898 (Gorochow et al., Nov. 26, 2020, “Layered Braided Aneurysm Treatment Device”), U.S. Pat. No. 11,413,046 (Xu et al., Aug. 16, 2022, “Layered Braided Aneurysm Treatment Device”), and U.S. patent application No. 20200367900 (Pedroso et al., Nov. 26, 2020, “Layered Braided Aneurysm Treatment Device With Corrugations”) disclose a tubular braid comprising an open end, a pinched end, and a predetermined shape; wherein, in the predetermined shape, the tubular braid comprises: a first segment extending from the open end to a first inversion, a second segment encircled by the open end such that the second segment is only partially surrounded by the first segment and extending from the first inversion to a second inversion, and a third segment surrounded by the second segment and extending from the second inversion to the pinched end.

U.S. patent application No. 20200155333 (Franano et al., May 21, 2020, “Ballstent Device and Methods of Use”) discloses a rounded, thin-walled, expandable metal structure and a flexible, elongated delivery device. U.S. patent application No. 20200187952 (Walsh et al., Jun. 18, 2020, “Intrasaccular Flow Diverter for Treating Cerebral Aneurysms”) and U.S. patent application 20220151632 (Walsh et al., May 19, 2022, “Intrasaccular Flow Diverter for Treating Cerebral Aneurysms”) disclose a stabilizing frame with two parts, the first part sized to anchor within the sac of the aneurysm and the exterior part sized to anchor against a region of the blood vessel wall adjacent the aneurysm neck.

U.S. patent application No. 20200187953 (Hamel et al., Jun. 18, 2020, “Devices, Systems, and Methods for the Treatment of Vascular Defects”) discloses a mesh comprising a first end portion, a second end portion, and a length extending between the first and second end portions, and a first lateral edge, a second lateral edge, and a width extending between the first and second lateral edges. U.S. patent application No. 20200205841 (Aboytes et al., Jul. 2, 2020, “Devices, Systems, and Methods for the Treatment of Vascular Defects”) and U.S. patent application No. 20210378681 (Aboytes et al., Dec. 9, 2021, “Devices, Systems, and Methods for the Treatment of Vascular Defects”) disclose aneurysm occlusion devices with a first configuration in which a first portion and a second portion are substantially linearly aligned and a second configuration in which the second portion at least partially overlaps the first portion.

U.S. patent application No. 20200281603 (Marchand et al., Sep. 10, 2020, “Filamentary Devices for Treatment of Vascular Defects”) discloses a permeable shell including a really swell polymer. U.S. patent application No. 20200289125 (Dholakia et al., Sep. 17, 2020, “Filamentary Devices Having a Flexible Joint for Treatment of Vascular Defects”) discloses an implant with a first permeable shell having a proximal end with a concave or recessed section and a second permeable shell having a convex section that mates with the concave or recessed section. U.S. patent application 20200289126 (Hewitt et al., Sep. 17, 2020, “Filamentary Devices for Treatment of Vascular Defects”), U.S. Pat. No. 11,317,921 (Hewitt et al., May 3, 2022, “Filamentary Devices for Treatment of Vascular Defects”), and U.S. patent application No. 20220257258 (Hewitt et al., Aug. 18, 2022, “Filamentary Devices for Treatment of Vascular Defects”) disclose a permeable shell or mesh with a stiffer proximal portion at the neck of an aneurysm.

U.S. patent application No. 20200305885 (Soto Del Valle et al, Oct. 1, 2020, “Aneurysm Treatment Device”) discloses an occlusion device that expands to form a cup shape within an aneurysm sac. U.S. patent application No. 20200305886 (Soto Del Valle et al, Oct. 1, 2020, “Aneurysm Treatment Device”) and U.S. patent application No. 20220225997 (Soto Del Valle et al., Jul. 21, 2022, “Aneurysm Treatment Device”) disclose a device with an expandable sack with a free open end and an elongated looping portion. U.S. patent application No. 20200367896 (Zaidat et al., Nov. 26, 2020, “Systems and Methods for Treating Aneurysms”) discloses an apparatus for treating an aneurysm in a blood vessel with a first tubular mesh having a first end and a second end coupled together at a proximal end of the occlusion element.

U.S. patent application No. 20200367904 (Becking et al., Nov. 26, 2020, “Multiple Layer Filamentary Devices for Treatment of Vascular Defects”) and U.S. patent application No. 20220022886 (Becking et al., Jan. 27, 2022, “Multiple Layer Filamentary Devices for Treatment of Vascular Defects”) disclose braid-balls suitable for aneurysm occlusion. U.S. patent application 20200367906 (Xu et al., Nov. 26, 2020, “Aneurysm Treatment With Pushable Ball Segment”) and U.S. patent application No. 20230016312 (Xu et al., Jan. 19, 2023, “Aneurysm Treatment with Pushable Implanted Braid”) disclose a braided implant with a retractable dual proximal layer. U.S. patent application No. 20200375606 (Lorenzo, Dec. 3, 2020, “Aneurysm Method and System”) discloses a braided implant which is invertible about the distal implant end.

U.S. patent application No. 20200375607 (Soto Del Valle et al., Dec. 3, 2020, “Aneurysm Device and Delivery System”) discloses a method of expanding mesh segments to form an outer occlusive sack and an inner occlusive sack. U.S. patent application No. 20200405347 (Walzman, Dec. 31, 2020, “Mesh Cap for Ameliorating Outpouchings”) discloses a self-expandable occluding device which covers the neck of an outpouching and serves as a permanent embolic plug. U.S. patent application 20210007755 (Lorenzo et al., Jan. 14, 2021, “Intrasaccular Aneurysm Treatment Device with Varying Coatings”) discloses an aneurysm intrasaccular implant with coated regions. U.S. Pat. No. 10,905,430 (Lorenzo et al., Feb. 2, 2021, “Aneurysm Device and Delivery System”) discloses an expandable segment which radially expands inside an outer occlusive sack.

U.S. patent application No. 20210052279 (Porter et al., Feb. 25, 2021, “Intra-Aneurysm Devices”) discloses a device including an upper member that sits against the dome of an aneurysm, a lower member that sits in the neck of the aneurysm, and a means of adjusting the overall dimensions of the device. U.S. patent application No. 20210085333 (Gorochow et al., Mar. 25, 2021, “Inverting Braided Aneurysm Treatment System and Method”), U.S. Pat. No. 11,278,292 (Gorochow et al., Mar. 22, 2022, “Inverting Braided Aneurysm Treatment System and Method”), and U.S. patent application 20220104829 (Gorochow et al., Apr. 7, 2022, “Inverting Braided Aneurysm Treatment System and Method”) disclose a tubular braid with an intrasaccular section, an intravascular section, a pinched section, and a predetermined shape. U.S. patent application No. 20210106338 (Gorochow, Apr. 15, 2021, “Spiral Delivery System for Embolic Braid”) discloses a braided implant having a spiral segment.

U.S. patent application No. 20210128160 (Li et al., May 6, 2021, “Systems and Methods for Treating Aneurysms”) discloses delivering an occlusive member to an aneurysm sac in conjunction with an embolic element. U.S. patent application No. 20210128160 (Li et al., May 6, 2021, “Systems and Methods for Treating Aneurysms”), U.S. patent application No. 20210128167 (Patel et al., May 6, 2021, “Systems and Methods for Treating Aneurysms”), U.S. patent application No. 20210128168 (Nguyen et al., May 6, 2021, “Systems and Methods for Treating Aneurysms”) and disclose delivering an occlusive member (e.g., an expandable braid) to an aneurysm sac in conjunction with an embolic element (e.g., coils, embolic material).

U.S. patent application No. 20210128161 (Nageswaran et al., May 6, 2021, “Aneurysm Treatment Device”) discloses an aneurysm treatment system including a conduit with a distal portion, a coupler slidably coupled to the distal portion, an occlusive member coupled to the coupler, and a securing member coupled to the conduit proximal to the coupler. U.S. patent application 20210128162 (Rhee et al., May 6, 2021, “Devices, Systems, and Methods for Treatment of Intracranial Aneurysms”) discloses introduction of an embolic element to a space between an occlusive member and an inner surface of the aneurysm wall. U.S. patent application No. 20210128162 (Rhee et al., May 6, 2021, “Devices, Systems, and Methods for Treatment of Intracranial Aneurysms”) discloses delivering an occlusive member to an aneurysm cavity and transforming a shape of the occlusive member within the cavity, including delivering an embolic element between the occlusive member and the aneurysm wall.

U.S. patent application No. 20210128165 (Pulugurtha et al., May 6, 2021, “Systems and Methods for Treating Aneurysms”) discloses an occlusive member configured to be positioned within an aneurysm sac and a distal conduit coupled to the occlusive member and having a first lumen extending therethrough. U.S. patent application No. 20210128165 (Pulugurtha et al., May 6, 2021, “Systems and Methods for Treating Aneurysms”) and U.S. Pat. No. 11,305,387 (Pulugurtha et al., Apr. 19, 2022, “Systems and Methods for Treating Aneurysms”) disclose a distal conduit coupled to an occlusive member with a first lumen extending therethrough and a proximal conduit with a second lumen extending therethrough. U.S. patent application No. 20210128167 (Patel et al., May 6, 2021, “Systems and Methods for Treating Aneurysms”) discloses delivering an occlusive member to an aneurysm sac in conjunction with an embolic element.

U.S. patent applications 20210128168 (Nguyen et al., May 6, 2021. “Systems and Methods for Treating Aneurysms”) and 20230023511 (Nguyen et al., Jan. 26, 2023, “Systems and Methods for Treating Aneurysms”) disclose delivering an occlusive member to an aneurysm sac in conjunction with an embolic element. U.S. patent application No. 20210128169 (Li et al., May 6, 2021, “Devices, Systems, and Methods for Treatment of Intracranial Aneurysms”) and U.S. patent application 20210153872 (Nguyen et al., May 27, 2021, “Devices, Systems, and Methods for Treatment of Intracranial Aneurysms”) disclose delivering an occlusive member to an aneurysm cavity and deforming a shape of the occlusive member via introduction of an embolic element to a space between the occlusive member and an inner surface of the aneurysm wall.

U.S. patent applications 20210128169 (Li et al., May 6, 2021, “Devices, Systems, and Methods for Treatment of Intracranial Aneurysms”), 20210153872 (Nguyen et al., May 27, 2021, “Devices, Systems, and Methods for Treatment of Intracranial Aneurysms”), 20230294223 (Li et al., Sep. 21, 2023, “Devices, Systems, and Methods for Treatment of Intracranial Aneurysms”), and 20230373040 (Nguyen et al., Nov. 23, 2023, “Devices, Systems, and Methods for Treatment of Intracranial Aneurysms”) disclose delivering an occlusive member to an aneurysm cavity and deforming a shape of the occlusive member via introduction of an embolic element to a space between the occlusive member and the aneurysm wall. U.S. patent application No. 20210129275 (Nguyen et al., May 6, 2021, “Devices, Systems, and Methods for Treating Aneurysms”) discloses methods of manufacturing an occlusive device including conforming a mesh to a forming assembly and setting a shape of the mesh based on the forming assembly.

U.S. patent applications 20210129275 (Nguyen et al., May 6, 2021, “Devices, Systems, and Methods for Treating Aneurysms”) and 20230311254 (Nguyen et al., Oct. 5, 2023, “Devices, Systems, and Methods for Treating Aneurysms”) disclose methods of manufacturing an aneurysm occlusion device including shaping a mesh with a forming assembly comprising multiple forming members, a mandrel, and/or one or more coupling elements. U.S. patent application No. 20210137526 (Lee et al., May 13, 2021. “Embolic Devices for Occluding Body Lumens”) discloses an embolic device with a first segment forming a first three-dimensional structure, wherein the first three-dimensional structure defines a cavity; and a second segment forming a second three-dimensional structure; wherein the cavity of the first three-dimensional structure is configured to accommodate at least a majority of the second three-dimensional structure.

U.S. patent application No. 20210145449 (Gorochow, May 20, 2021, “Implant Delivery System with Braid Cup Formation”) discloses an implant system with an engagement wire, a pull wire, and a braided implant having a distal ring thereon. U.S. Pat. No. 11,013,516 (Franano et al., May 25, 2021, “Expandable Body Device and Method of Use”) discloses a single-lobed, thin-walled, expandable body comprising gold, platinum, or silver. U.S. patent application No. 20210169495 (Gorochow et al., Jun. 10, 2021, “Intrasaccular Inverting Braid with Highly Flexible Fill Material”) and U.S. Pat. No. 11,602,350 (Gorochow et al., Mar. 14, 2023, “Intrasaccular Inverting Braid with Highly Flexible Fill Material”) disclose a tubular braided implant which is delivered as a single layer braid, inverted into itself during deployment to form at least two nested sacks and includes additional braid material that can fill the innermost sack.

U.S. patent application No. 20210169498 (Gorochow, Jun. 10, 2021, “Delivery of Embolic Braid”) discloses a braided implant delivery system which attaches a braided implant having a band to a delivery tube, positions the braided implant within an aneurysm, and then releases the band from the delivery tube. U.S. Pat. No. 11,033,275 (Franano et al., Jun. 15, 2021, “Expandable Body Device and Method of Use”) discloses devices, designs, methods of manufacturing and using hollow gold structures that can be folded, wrapped, and compressed. U.S. patent application No. 20210177429 (Lorenzo, Jun. 17, 2021, “Aneurysm Method and System”) discloses a vaso-occlusive device with at least two nested sacks.

U.S. patent application No. 20210186518 (Gorochow et al., Jun. 24, 2021, “Implant Having an Intrasaccular Section and Intravascular Section”) and U.S. Pat. No. 11,457,926 (Gorochow et al., Oct. 4, 2022. “Implant Having an Intrasaccular Section and Intravascular Section”) disclose a tubular braid with an intrasaccular section, an intravascular section, a pinched section, and a predetermined shape. U.S. Pat. No. 11,051,825 (Gorochow, Jul. 6, 2021, “Delivery System for Embolic Braid”) discloses a braided implant which is attached to a releasing component that can be detachably engaged with a delivery tube and a pull wire. U.S. Pat. No. 11,058,430 (Gorochow et al., Jul. 13, 2021, “Aneurysm Device and Delivery System”) discloses a braid with a proximal expandable portion for positioning inside an aneurysm and sealing across the neck of the aneurysm.

U.S. Pat. No. 11,058,431 (Pereira et al., Jul. 13, 2021, “Systems and Methods for Treating Aneurysms”) discloses an inverted mesh tube having an outer layer and an inner layer, wherein the outer layer transitions to the inner layer at an inversion fold located at or adjacent to the distal end of the occlusion element. U.S. patent application No. 20210228214 (Bowman et al., Jul. 29, 2021, “Devices for Vascular Occlusion”) discloses a method of using and delivering an occlusive device. U.S. Pat. No. 11,076,861 (Gorochow et al., Aug. 3, 2021, “Folded Aneurysm Treatment Device and Delivery Method”) discloses an implant with a fold which defines an annular ridge and a radiopaque marker band.

U.S. patent application No. 20210244420 (Aboytes et al., Aug. 12, 2021, “Devices and Methods for the Treatment of Vascular Defects”) discloses aneurysm occlusion devices with a first configuration in which a first portion and a second portion are substantially linearly aligned and a second configuration in which the second portion at least partially overlaps the first portion. U.S. patent application No. 20210275187 (Franano et al., Sep. 9, 2021, “Expandable Body Device and Method of Use”) discloses medical devices comprising a single-lobed, thin-walled, expandable body. U.S. patent application No. 20210275779 (Northrop, Sep. 9, 2021, “Actuating Elements for Bending Medical Devices”) discloses an actuating element causes a tube to bend.

U.S. patent application No. 20210282784 (Sepetka et al., Sep. 16, 2021, “Occlusive Device”) discloses a device comprising a plurality of braided wires and an embolic coil. U.S. patent application No. 20210282785 (Dholakia et al., Sep. 16, 2021, “Devices Having Multiple Permeable Shells for Treatment of Vascular Defects”) a device with a plurality of permeable shells connected by a plurality of coils. U.S. patent application No. 20210282789 (Vu et al., Sep. 16, 2021, “Multiple Layer Devices for Treatment of Vascular Defects”) discloses a first permeable shell and a second permeable shell, where the second permeable shell sits within an interior cavity of the first permeable shell. U.S. Pat. No. 11,123,077 (Lorenzo et al., Sep. 21, 2021, “Intrasaccular Device Positioning and Deployment System”) discloses implant deployment systems including a braided implant that can be detachably attached to a delivery tube by an expansion ring.

U.S. patent application No. 20210330331 (Lorenzo, Oct. 28, 2021, “Aneurysm Occlusion Device”) and U.S. Pat. No. 11,154,302 (Lorenzo et al., Oct. 26, 2021, “Aneurysm Occlusion Device”) disclose an occlusion device with a substantially annular body disposed on the proximal end region of the device. U.S. patent application No. 20210338247 (Gorochow, Nov. 4, 2021, “Double Layer Braid”) discloses a double layered braid for treating an aneurysm. U.S. patent application No. 20210338250 (Gorochow et al., Nov. 4, 2021, “Intrasaccular Flow Diverter”) and U.S. Pat. No. 11,523,831 (Gorochow et al., Dec. 13, 2022, “Intrasaccular Flow Diverter”) disclose an interior fill braid physically which is inverted over itself to form a proximal inverted end and an opposite free end and a dome braid disposed distally of and secured to the interior fill braid.

U.S. Pat. No. 11,166,731 (Wolfe et al., Nov. 9, 2021. “Systems and Methods for Treating Aneurysms”) discloses an inverted mesh tube having an outer layer and an inner layer, the outer layer transitioning to the inner layer at an inversion fold. U.S. patent application No. 20210346032 (Patterson et al., Nov. 11, 2021, “Devices for Treatment of Vascular Defects”) discloses an expandable stent for placement in a parent vessel proximal, near, or adjacent an aneurysm. U.S. patent application No. 20210353299 (Hamel et al., Nov. 18, 2021, “Devices, Systems, and Methods for the Treatment of Vascular Defects”) discloses a mesh that is curved along its length with an undulating contour across at least a portion of one or both of its length or its width.

U.S. patent application No. 20210353300 (Kottenmeier et al., Nov. 18, 2021, “Systems and Methods for Treatment of Defects in the Vasculature”) discloses aneurysm occlusion methods and systems including an expandable stent. U.S. Pat. No. 11,179,159 (Cox et al., Nov. 23, 2021, “Methods and Devices for Treatment of Vascular Defects”) discloses a device comprising a first hub, a second hub, a support structure having a longitudinal axis, the support structure disposed between the first hub and the second hub, the support structure including a plurality of struts, and a layer of material disposed over the plurality of struts, wherein the first hub is cylindrical and connected to an end of each of the struts of the plurality of struts. U.S. Pat. No. 11,185,335 (Badruddin et al., Nov. 30, 2021, “System for and Method of Treating Aneurysms”) discloses an apparatus for treating an aneurysm with an occlusion element disposed on a wire, wherein the occlusion element includes a cover for covering a neck of an aneurysm and an inner anchoring member.

U.S. Pat. No. 11,202,636 (Zaidat et al., Dec. 21, 2021, “Systems and Methods for Treating Aneurysms”), U.S. patent application No. 20220022884 (Wolfe et al., Jan. 27, 2022, “Systems and Methods for Treating Aneurysms”), and U.S. patent application No. 20220211383 (Pereira et al., Jul. 7, 2022, “Systems and Methods for Treating Aneurysms”) disclose an apparatus for treating an aneurysm including an occlusion element configured to be releasably coupled to an elongate delivery shaft and a distal end, a proximal end, and a longitudinal axis extending between the distal end and the proximal end. U.S. patent application No. 20220031334 (Aguilar, Feb. 3, 2022, “Expandable Devices for Treating Body Lumens”) discloses an occlusive device comprising an expandable mesh including an outer mesh and an inner mesh disposed within the outer mesh, a connection portion positioned at or within an inner cavity of the inner mesh and configured to be detachably coupled to a delivery member.

U.S. patent application No. 20220031334 (Aguilar, Feb. 3, 2022, “Expandable Devices for Treating Body Lumens”) discloses an occlusive device comprising an expandable mesh including an outer mesh and an inner mesh disposed within the outer mesh. U.S. patent application No. 20220039804 (Rangwala et al., Feb. 10, 2022, “Flow-Diverting Implant and Delivery Method”) discloses a saddle-shaped braided mesh diverter that covers the neck of an aneurysm. U.S. patent application 20220054141 (Zaidat et al., Feb. 24, 2022, “Systems and Methods for Treating Aneurysms”) discloses an apparatus for treating an aneurysm in a blood vessel with a first tubular mesh having a first end and a second end coupled together at a proximal end of the occlusion element.

U.S. patent application No. 20220087681 (Xu et al., Mar. 24, 2022, “Inverting Braided Aneurysm Implant with Dome Feature”) discloses an implant with a dome feature configured to press into aneurysm walls near the aneurysm's dome and facilitate securement of the braid across the aneurysm's neck. U.S. Pat. No. 11,337,706 (Soto Del Valle et al., May 24, 2022, “Aneurysm Treatment Device”) discloses an implant having an elongated portion and an expandable braided sack portion. U.S. patent application No. 20220175389 (Wallace et al., Jun. 9, 2022, “Vaso-Occlusive Devices Including a Friction Element”) discloses a vaso-occlusive implant with a friction element between a soft braided member and a coil.

U.S. patent application No. 20220192678 (Hewitt et al., Jun. 23, 2022, “Filamentary Devices for Treatment of Vascular Defects”) discloses an implant having a first permeable shell having a proximal hub and an open distal end and a second permeable shell having a distal hub and an open proximal end. U.S. patent application No. 20220202425 (Gorochow et al., Jun. 30, 2022, “Semispherical Braided Aneurysm Treatment System and Method”) discloses a tubular braid with three segments and two inversions, one of the three segments extending between the two inversions and forming a sack. U.S. patent application No. 20220249098 (Milhous et al., Aug. 11, 2022, “Filamentary Devices for Treatment of Vascular Defects”) discloses a permeable implant with a plurality of scaffolding filaments. U.S. patent application No. 20220257260 (Hewitt et al., Aug. 18, 2022, “Filamentary Devices for Treatment of Vascular Defects”) discloses an implant having multiple mesh layers.

U.S. Pat. No. 11,426,175 (Morita et al., Aug. 30, 2022, “Expansile Member”) discloses an occlusive system comprising: a catheter; a shell deliverable through the catheter, a delivery pusher detachably connected to the shell and configured to navigate the shell through the catheter, wherein the shell has a globular shaped portion. U.S. Pat. No. 11,471,162 (Griffin, Oct. 18, 2022, “Occlusion Device”) discloses an occlusion device for implantation into a body lumen or aneurysm which has a continuous compressible mesh structure comprising axial mesh carriages configured end to end, wherein each end of each carriage is a pinch point in the continuous mesh structure. U.S. patent application No. 20220330947 (Henkes et al., Oct. 20, 2022, “Implant for the Treatment of Aneurysms”) discloses an implant which is rolled up relative to a radial axis in order to form a balled-up configuration.

U.S. Pat. No. 11,497,504 (Xu et al., Nov. 15, 2022, “Aneurysm Treatment with Pushable Implanted Braid”) discloses a braided implant with a retractable dual proximal layer. U.S. Pat. No. 11,498,165 (Patel et al., Nov. 15, 2022, “Systems and Methods for Treating Aneurysms”) discloses an occlusive implant with a conduit which receives a liquid embolic. U.S. patent application 20220370078 (Chen et al., Nov. 24, 2022, “Vaso-Occlusive Devices”) discloses a vaso-occlusive structure made with a gold-platinum-tungsten alloy. U.S. Pat. No. 11,517,321 (Mauger et al., Dec. 6, 2022, “System and Methods for Embolized Occlusion of Neurovascular Aneurysms”) discloses an occlusion device with a reinforcing portion with no porosity. U.S. patent application 20230017150 (Lee et al., Jan. 19, 2023, “Hydrogel Stent and Embolization Device for Cerebral Aneurysm”) discloses a hydrogel stent for occluding a cerebral aneurysm.

U.S. Pat. No. 11,559,309 (Rangwala et al., Jan. 24, 2023, “Filamentary Devices for Treatment of Vascular Defects”) discloses a permeable implant whose proximal portion is stiffer. U.S. patent application No. 20230031965 (Sivapatham, Feb. 2, 2023, “Intrasaccular Stent Device for Aneurysm Treatment”) discloses a system for treating an aneurysm in a blood vessel comprising a catheter, a guidewire, a delivery wire, an intrasaccular stent/retaining device removably attached to the delivery wire, and endovascular coiling. U.S. patent application No. 20230039246 (Hossan et al., Feb. 9, 2023, “Non-Braided Biodegradable Flow Diverting Device for Endovascular Treatment of Aneurysm and Associated Fabrication Method”) discloses a biodegradable flow-diverting device that regulates blood flow into an aneurysmal sac.

U.S. patent application No. 20230039773 (Monstadt et al., Feb. 9, 2023, “Implant for Treating Aneurysms”) discloses an implant which is preset to a specific structure. U.S. Pat. No. 11,583,282 (Gorochow et al., Feb. 21, 2023, “Layered Braided Aneurysm Treatment Device”) discloses a method for shaping a tubular braid into a predetermined shape. U.S. Pat. No. 11,589,872 (Mauger, Feb. 28, 2023, “Vascular Occlusion Devices Utilizing Thin Film Nitinol Foils”) discloses an implantable occlusion device wherein mesh components are wrapped around a support structure and slot that enables a disc to be wrapped around the support structure with overlapping portions. U.S. patent application 20230061363 (Yee et al., Mar. 2, 2023, “Embolic Device with Improved Neck Coverage”) discloses an embolic device with a flexible structure which has a series of alternating narrow portions and link portions.

U.S. Pat. No. 11,596,412 (Xu et al., Mar. 7, 2023, “Aneurysm Device and Delivery System”) discloses a braid with a proximal portion which goes across an aneurysm neck and an expandable distal portion. U.S. Pat. No. 11,607,226 (Pedroso et al., Mar. 21, 2023, “Layered Braided Aneurysm Treatment Device with Corrugations”) discloses an implant with a proximal inversion and two segments. U.S. patent application No. 20230107778 (Cox et al., Apr. 6, 2023, “Methods and Devices for Treatment of Vascular Defects”) discloses an expandable body comprising a plurality of elongate filamentary elements each having a first end and a second end, wherein the elements extend from a first end of the device to a second end of the device and back to the first end of the device.

U.S. patent application No. 20230114169 (Hewitt et al., Apr. 13, 2023, “Devices for Treatment of Vascular Defects”) discloses a permeable woven implant with a radially-constrained state for delivery within a catheter and an expanded state thereafter. U.S. patent application No. 20230157696 (Carrillo, May 25, 2023, “Aneurysm Treatment Device and Associated Systems and Methods of Use”) discloses an aneurysm treatment device with a tip portion, a body portion, and a base portion. U.S. patent application No. 20230165587 (Carrillo, Jun. 1, 2023, “Expandable Devices for Treating Body Lumens”) discloses an expandable cage with a plurality of mesh stents which receives embolic material therein. U.S. patent application No. 20230190292 (Choubey et al., Jun. 22, 2023, “Occlusive Devices with Petal-Shaped Regions for Treating Vascular Defects”) discloses an occlusive device for treating an aneurysm with a mesh formed from a tubular braid, including a petal-shaped region formed from a flattened section of the tubular braid.

U.S. Pat. No. 11,685,007 (Li et al., Jun. 27, 2023, “Devices, Systems, and Methods for Treatment of Intracranial Aneurysms”) discloses delivering an occlusive member to an aneurysm cavity and deforming a shape of the occlusive member via introduction of an embolic element to a space between the occlusive member and the aneurysm wall. U.S. patent application No. 20230225735 (Kulak et al., Jul. 20, 2023, “Expandable Devices for Treating Body Lumens”) discloses an occlusive device comprising a mesh having a low-profile state for intravascular delivery to the aneurysm and a deployed state, wherein the mesh comprises a tubular mesh configured to curve along its longitudinal dimension when implanted in an aneurysm cavity. U.S. patent application 20230225735 (Kulak et al., Jul. 20, 2023, “Expandable Devices for Treating Body Lumens”) discloses a tubular mesh which curves along its longitudinal dimension when implanted in an aneurysm cavity.

U.S. patent application No. 20230240686 (Ashby et al., Aug. 3, 2023, “Occlusive Devices with Spiral Struts for Treating Vascular Defects”) discloses an occlusive device with a plurality of spiral struts. U.S. patent application No. 20230252631 (Kashyap et al., Aug. 10, 2023, “Neural Network Apparatus for Identification, Segmentation, and Treatment Outcome Prediction for Aneurysms”) discloses using medical imaging and a neural network to predict outcomes from the potential use of one or more different intrasaccular implant devices. U.S. patent application No. 20230263528 (Jones, Aug. 24, 2023, “Intrasaccular Flow Diverter and Related Methods”) discloses an intrasaccular flow diverter with a plurality of wires which are coiled to form a collapsible, substantially spherical frame.

U.S. patent application No. 20230277184 (Rashidi et al., Sep. 7, 2023, “Occlusive Devices with Thrombogenic Inserts”) discloses an expandable mesh which spans a neck of the aneurysm with an insert configured to promote thrombosis. U.S. patent application No. 20230277184 (Rashidi, Sep. 7, 2023, “Occlusive Devices With Thrombogenic Inserts”) discloses an occlusive device for treating an aneurysm includes an expandable mesh configured to span a neck of the aneurysm, wherein expandable mesh can include an upper wall, a lower wall, and an insert between the upper and lower walls. U.S. patent application No. 20230285031 (Mayer et al, Sep. 14, 2023, “Device for Restricting Blood Flow to Aneurysms”) discloses a non-occlusive device with a coilable section and a docking section.

U.S. patent application No. 20230301660 (Sonmez et al., Sep. 28, 2023, “Systems and Methods for Treating Aneurysms”) discloses delivering an occlusive member to an aneurysm sac in conjunction with an embolic element. U.S. patent application No. 20230397913 (Greenwood et al., Dec. 14, 2023, “Friction Fit Endovascular Implant Detachment Mechanism”) discloses an endovascular implant including an open end and a pinched end, a connector positioned approximate the pinched end, a lock wire, and an outer coil surrounding the lock wire. U.S. patent application No. 20230404590 (Xu et al., Dec. 21, 2023, “Inverting Braided Aneurysm Treatment System Having a Semi-Frustoconically-Shaped Portion”) discloses a tubular braid with a first segment extending from an open end to a first inversion, a second segment extending from the first inversion to a second inversion, and a third segment which extends from the second inversion to a pinched end.

U.S. patent application No. 20230404592 (Nageswaran, Dec. 21, 2023, “Implant with Intrasaccular and Intravascular Portions and Related Technology”) discloses technology configured for treating an aneurysm at a treatment location within a patient's vasculature at which first, second, and third blood vessels converge. U.S. patent application No. 20240016499 (Xu et al., Jan. 18, 2024, “Braided Implant with Atraumatic End”) discloses a tubular braid including an open end and a pinched end. U.S. patent application No. 20240032941 (Shimizu et al., Feb. 1, 2024, “Embolic Material Delivery Device and Related Technology”) discloses an elongate conduit body defining an axial lumen through which the conduit body is configured to convey liquid embolic material toward the aneurysm. U.S. patent application No. 20240050099 (Pecor et al., Feb. 15, 2024, “Occlusive Devices for Treating Vascular Defects and Associated Systems and Methods”) discloses a plurality of braided filaments configured to be implanted in an aneurysm cavity.

U.S. patent application No. 20240065699 (Sloss et al., Feb. 29, 2024, “Twister Implant Detachment Mechanism”) discloses an endovascular implant detachment mechanism with a connector, a push wire, and one or more lock wires. U.S. patent application No. 20240075565 (Li et al., Mar. 7, 2024, “Systems and Methods for Treating Aneurysms”) discloses delivering an occlusive member to an aneurysm sac in conjunction with an embolic element. U.S. patent application No. 20240099720 (Greenwood et al., Mar. 28, 2024, “Braided Implant with Detachment Mechanism”) discloses an occlusive device with a segment including an open end and a proximal end, as well as a push wire that is positioned proximal of the proximal end. U.S. patent application No. 20240108354 (Xu et al., Apr. 4, 2024, “Braided Implant with Integrated Embolic Coil”) discloses a tubular braid with a band positioned near a pinched end which is attached to an embolic coil.

### SUMMARY OF THE INVENTION

This invention is a torus or torus-section shaped intrasaccular aneurysm occlusion device. A torus shape can be geometrically modeled by revolving a convex shape in three-dimensional space around an axis of revolution which is coplanar with the convex shape. A torus section shape can be geometrically modeled by revolving a section (e.g. half or three-quarters) of a convex shape in three-dimensional space around an axis of revolution which is coplanar with the convex shape. In some examples, a convex shape which is revolved can be a circle, an ellipse, or half of a yin-yang symbol. In an example, an torus or torus section shaped aneurysm occlusion device can further comprise one or more embolic members or embolic material which is inserted into the aneurysm sac through a central opening in the neck bridge.

There are several advantages of a torus or torus-section shape for a neck bridge, as compared to other shapes such as a hemispherical, bowl, or cup shape. For example, a torus or torus-section shape forms a central funnel or hyperboloid shaped opening whose walls guide the insertion of a catheter through the neck bridge for delivering embolic members or embolic material into the aneurysm sac. Also, compared to a hemisphere, bowl, or cup shape, a torus shape can provide greater radial-resistance than a hemispherical, bowl, or cup shape. This can help to prevent a torus or torus-section neck bridge from slipping out from an aneurysm sac.

### BRIEF INTRODUCTION TO THE FIGURES

FIG. 1 shows a wire-mesh view of an aneurysm occlusion device with a torus shape which is modeled by revolving a circle.

FIG. 2 shows how the shape of the device in FIG. 1 is formed by revolving the circle.

FIG. 3 shows a wire-mesh view of an aneurysm occlusion device with a torus shape which is modeled by revolving an ellipse whose longitudinal axis is parallel to an axis of revolution.

FIG. 4 shows how the shape of the device in FIG. 3 is formed by revolving the ellipse.

FIG. 5 shows a wire-mesh view of an aneurysm occlusion device with a torus shape which is modeled by revolving an ellipse whose longitudinal axis is perpendicular to an axis of revolution.

FIG. 6 shows how the shape of the device in FIG. 5 is formed by revolving the ellipse.

FIG. 7 shows a wire-mesh view of an aneurysm occlusion device with a torus section shape which is modeled by revolving half of a circle.

FIG. 8 shows how the shape of the device in FIG. 7 is formed by revolving the half circle.

FIG. 9 shows a wire-mesh view of an aneurysm occlusion device with a torus section shape which is modeled by revolving half of an ellipse whose longitudinal axis is parallel to an axis of revolution.

FIG. 10 shows how the shape of the device in FIG. 9 is formed by revolving the half ellipse.

FIG. 11 shows a wire-mesh view of an aneurysm occlusion device with a torus section shape which is modeled by revolving half of an ellipse whose longitudinal axis is perpendicular to an axis of revolution.

FIG. 12 shows how the shape of the device in FIG. 11 is formed by revolving the half ellipse.

FIG. 13 shows a wire-mesh view of an aneurysm occlusion device with a torus shape which is modeled by revolving half of a yin-yang symbol.

FIG. 14 shows how the shape of the device in FIG. 13 is formed by revolving half of the yin-yang symbol.

FIG. 15 shows a wire-mesh view of an aneurysm occlusion device with a torus shape which is modeled by revolving an ellipse, wherein an extension of the longitudinal axis of the ellipse intersects the axis of revolution at an acute angle.

FIG. 16 shows how the shape of the device in FIG. 15 is formed by revolving the ellipse.

FIG. 17 shows a wire-mesh view of an aneurysm occlusion device with a torus section shape which is modeled by revolving a section of a circle, wherein the section is a circle with a gap between the 2 o'clock and 4 o'clock coordinates.

FIG. 18 shows how the shape of the device in FIG. 17 is formed by revolving the section of the circle.

FIG. 19 shows the section of the circle in FIG. 18, including display of clock coordinates.

FIG. 20 shows a wire-mesh view of an aneurysm occlusion device with a torus section shape which is modeled by revolving a section of a circle, wherein the section is a circle with a gap between the 1 o'clock and 3 o'clock coordinates.

FIG. 21 shows how the shape of the device in FIG. 20 is formed by revolving the section of the circle.

FIG. 22 shows the section of the circle in FIG. 21, including display of clock coordinates.

FIG. 23 shows a wire-mesh view of an aneurysm occlusion device with a torus section shape which is modeled by revolving a section of a circle, wherein the section is a circle with a gap between the 9 o'clock and 12 o'clock coordinates.

FIG. 24 shows how the shape of the device in FIG. 23 is formed by revolving the section of the circle.

FIG. 25 shows the section of the circle in FIG. 24, including display of clock coordinates.

FIG. 26 shows a wire-mesh view of an aneurysm occlusion device with a torus section shape which is modeled by revolving a section of a circle, wherein the section is a circle with a gap between the 7 o'clock and 11 o'clock coordinates.

FIG. 27 shows how the shape of the device in FIG. 26 is formed by revolving the section of the circle.

FIG. 28 shows the section of the circle in FIG. 27, including display of clock coordinates.

FIG. 29 shows how a torus-shaped neck bridge can be made by curving the longitudinal axis of a tubular mesh and then connecting the ends of the mesh together.

FIG. 30 shows how a torus-shaped neck bridge can be made by inverting the ends of tubular mesh and then connecting these ends together.

FIG. 31 shows a torus section shaped intrasaccular aneurysm occlusion device with reinforcing rings around its widest circumference and its central opening.

FIG. 32 shows a torus section shaped intrasaccular aneurysm occlusion device with an undulating reinforcing ring around its widest circumference and another reinforcing ring around its central opening.

FIG. 33 shows a torus section shaped intrasaccular aneurysm occlusion device with a valve on its central opening.

FIG. 34 shows a wire-mesh view of an intrasaccular aneurysm occlusion device comprising a proximal torus or torus-section shaped neck bridge and a distal globular mesh or net, wherein a portion of the globular mesh or net is nested within the neck bridge.

FIG. 35 shows a wire-mesh view of an intrasaccular aneurysm occlusion device comprising a proximal torus or torus-section shaped neck bridge and a distal globular mesh or net, wherein the neck bridge is inside the mesh or net.

FIG. 36 shows an intrasaccular aneurysm occlusion device comprising a catheter, a torus shaped neck bridge, and a longitudinal embolic member which is inserted through the neck bridge.

FIG. 37 shows an intrasaccular aneurysm occlusion device comprising a catheter, a torus section shaped neck bridge, and a longitudinal embolic member which is inserted through the neck bridge.

### DETAILED DESCRIPTION OF THE FIGURES

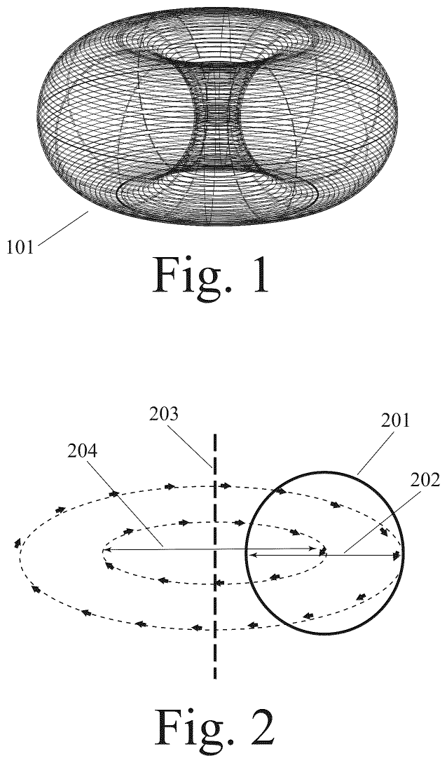

Before discussing the specific embodiments of this invention which are shown in FIGS. 1 through 37, this disclosure provides an introductory section which covers some of the general concepts, components, and methods which comprise this invention. Where relevant, these concepts, components, and methods can be applied as variations to the examples shown in FIGS. 1 through 37 which are discussed afterwards.

In an example, an intrasaccular aneurysm occlusion device can comprise a torus or torus-section shaped neck bridge which is configured to be inserted and radially-expanded within an aneurysm sac to cover the neck of the aneurysm. In an example, embolic members (e.g. embolic coils, string-of-pearls embolic strands, hydrogels, microbeads, or microsponges) or embolic material (e.g. congealing liquid or gel) can be inserted into the aneurysm sac through a central funnel or hyperboloid shaped opening in the neck bridge.

There are several reasons why a torus or torus-section shape for a neck bridge can be advantageous compared to other shapes such as a hemisphere, bowl, or cup. For example, a torus or torus-section shape automatically forms a central funnel or hyperboloid shaped opening which can guide the insertion of a catheter for embolic members or material into the aneurysm sac. The inwardly-curved walls of the funnel shape help to guide the catheter toward the opening more easily than a hole poked through the proximal surface of a hemisphere, bowl, or cup. Also, such an opening is more structurally-secure than a hole poked through a hemisphere, bowl, or cup.

As another advantage compared to a hemisphere, bowl, or cup shape, a torus shape can provide greater radial-resistance. This can help to prevent a torus or torus-section neck bridge from slipping out from the aneurysm sac, as compared to a hemispherical, bowl, or cup shaped neck bridge. Another advantage of a torus or torus-section shape for a neck bridge is that the local concavity at the proximal center of the torus reduces protrusion of the neck bridge into the parent vessel. When a torus or torus-section shaped neck bridge is combined with insertion of embolic members through the neck bridge into the aneurysm sac, it can more effectively fill an aneurysm sac with an irregularly-shaped dome than is possible with a single-piece globular (e.g. spherical) mesh.

A torus shape can be geometrically-modeled by revolving a convex shape (e.g. a circle, an ellipse, an egg shape, or a convex polygon) in three-dimensional space around an axis of revolution which is coplanar with the convex shape. One of the most common torus shapes is formed by revolving a circle in three-dimensional space around an axis of revolution which does not intersect the circle, creating a doughnut shape. As referenced herein, the term “convex shape diameter” with respect to a torus shape is defined as the widest diameter of the convex shape which is perpendicular to the axis of revolution. As referenced herein, the term “revolution diameter” with respect to a torus shape is the diameter of the path formed by the center of the convex shape, as the convex shape is revolved in three-dimensional space. In an example, the revolution diameter can be greater than the convex shape diameter. Although this geometric modeling is useful for defining the shape of a torus-shaped neck bridge, a torus-shaped neck bridge can be made by methods other than revolving a convex shape.

A torus-section shape can be geometrically-modeled by revolving a section (e.g. a selected percentage or clock-hour interval of the perimeter) of a convex shape (e.g. a circle, an ellipse, an egg shape, or a convex polygon) in three-dimensional space around an axis of revolution which is coplanar with the convex shape. As referenced herein, the term “convex shape diameter” with respect to a torus-section shape is defined as the widest diameter of the full perimeter (e.g. not just the section) of the convex shape which is perpendicular to the axis of revolution. As referenced herein, the term “revolution diameter” with respect to a torus-section shape is defined as the diameter of the path formed by the center of the full convex shape (e.g. not just the section) as the section of the convex shape is revolved in three-dimensional space. In an example, the revolution diameter can be greater than the convex shape diameter. Although this geometric modeling is useful for defining the shape of a torus-section-shaped neck bridge, a torus-section-shaped neck bridge can be made by methods other than revolving a section of a convex shape.

In an example, an intrasaccular aneurysm occlusion device can comprise: a torus-shaped neck bridge which is configured to be inserted into an aneurysm sac and then expanded to cover the neck of the aneurysm, wherein the shape of the neck bridge can be modeled by revolving a convex shape in three-dimensional space around an axis of revolution which is coplanar with the convex shape, wherein a convex shape diameter is the widest diameter of the convex shape which is perpendicular to the axis of revolution, wherein a revolution diameter is the widest diameter of the circle formed by the path of the center of the convex shape as the convex shape is revolved in three-dimensional space, and wherein the revolution diameter is greater than the convex shape diameter. In an example, the device can further comprise one or more embolic members or embolic material which is inserted into the aneurysm sac through a central opening in the neck bridge. In an example, the revolution diameter can be 5% to 30% greater than the convex shape diameter.

In an example, the convex shape can be a circle. In an example, the convex shape can be an ellipse with a longitudinal axis which is perpendicular to the axis of revolution. In an example, the convex shape can be an ellipse with a longitudinal axis which is parallel to the axis of revolution. In an example, the convex shape can be an ellipse wherein an extension of a longitudinal axis of the ellipse intersects the axis of revolution at an acute angle. In an example, the convex shape is the perimeter of the yin portion or the yang portion of a yin-yang symbol. In an example, the convex shape can be the perimeter of a teardrop shape.

In an example, an intrasaccular aneurysm occlusion device can comprise: a torus-section-shaped neck bridge which is configured to be inserted into an aneurysm sac and then expanded to cover the neck of the aneurysm, wherein the shape of the neck bridge can be modeled by revolving a section of a convex shape in three-dimensional space around an axis of revolution which is coplanar with the convex shape, wherein a convex shape diameter is the widest diameter of the convex shape which is perpendicular to the axis of revolution, wherein a revolution diameter is the widest diameter of the circle formed by the path of the center of the convex shape as the convex shape is revolved in three-dimensional space, and wherein the revolution diameter is greater than the convex shape diameter. In an example, the device can further comprise one or more embolic members or embolic material which is inserted into the aneurysm sac through a central opening in the neck bridge. In an example, the revolution diameter can be 5% to 30% greater than the convex shape diameter.

In an example, the section of a convex shape can be a semi-circle. In an example, the section of a convex shape can be the perimeter of a circle with a gap within a portion between 9 o'clock and 3 o'clock coordinates. In an example, the section of a convex shape can be the perimeter of a circle with a gap within a portion between 1 o'clock and 5 o'clock coordinates. In an example, the section of a convex shape can be a section of an ellipse with a longitudinal axis which is perpendicular to the axis of revolution. In an example, the section of a convex shape can be a section of an ellipse with a longitudinal axis which is parallel to the axis of revolution. In an example, the section of a convex shape can be a section of an ellipse wherein an extension of a longitudinal axis of the ellipse intersects the axis of revolution at an acute angle. In an example, the section of the convex shape can be the perimeter of an ellipse with a gap within a portion between 1 o'clock and 5 o'clock coordinates.

In an example, an intrasaccular aneurysm occlusion device can comprise: a longitudinal catheter that is configured to be inserted into a blood vessel, wherein this blood vessel is the parent vessel from which an aneurysm has formed; a longitudinal flexible embolic member that is configured to travel through the longitudinal lumen and be inserted into the aneurysm sac; and an expandable toroidal member that is configured to travel through the longitudinal lumen, be inserted into the aneurysm sac, and be expanded within the aneurysm sac; wherein this toroidal member is configured to substantially occlude the aneurysm neck after it is expanded; wherein the longitudinal flexible embolic member is inserted into the aneurysm sac through a central opening in the expandable toroidal member; and wherein the expandable toroidal member prevents the longitudinal flexible member from protruding into the parent vessel of the aneurysm.

In an example, an intrasaccular aneurysm occlusion device can comprise: a catheter which is configured to be inserted into an aneurysm sac; a torus or torus-section shaped neck bridge which is configured to be delivered through the catheter into the aneurysm sac and then radially-expanded to cover the neck of the aneurysm, wherein there is a central proximal opening in the neck bridge; and embolic members or material which are inserted through the central proximal opening into the aneurysm sac.

In an example, an intrasaccular aneurysm occlusion device can comprise: a catheter which is configured to be inserted into an aneurysm sac; a torus-shaped neck bridge (e.g. stent, lattice, mesh, or framework) which is configured to be delivered through the catheter into the aneurysm sac and self-expand within the aneurysm sac to a diameter which is larger than the diameter of the aneurysm neck; one or more embolic members (e.g. embolic coils, string-of-pearls embolic strands, hydrogels, microbeads, or microsponges) which are inserted into the aneurysm sac; a central proximal opening in the neck bridge through which the embolic members are inserted into the aneurysm sac; and a closure mechanism which closes the central proximal opening after the embolic members have been inserted into the aneurysm sac.

In an example, an intrasaccular aneurysm occlusion device can comprise: a catheter which is configured to be inserted into an aneurysm sac; a torus-shaped neck bridge (e.g. stent, lattice, mesh, or framework) which is configured to be delivered through the catheter into the aneurysm sac and self-expand within the aneurysm sac to a diameter which is larger than the diameter of the aneurysm neck; embolic material (e.g. congealing liquid or gel) which is inserted into the aneurysm sac; a central proximal opening in the neck bridge through which the embolic material is inserted into the aneurysm sac; and a closure mechanism which closes the central proximal opening after the embolic material has been inserted into the aneurysm sac.

In an example, an intrasaccular aneurysm occlusion device can comprise: a catheter that is configured to be inserted into a blood vessel, wherein this blood vessel is the parent vessel from which an aneurysm has formed; one or more longitudinal flexible embolic members (e.g. coils or string-of-pearls embolic strands) that travel through the catheter and are inserted into the aneurysm sac; and a torus or torus-section shaped neck bridge that travels through the catheter, is inserted into the aneurysm sac, and is expanded within the aneurysm sac; wherein the neck bridge is configured to substantially occlude the aneurysm neck after it is expanded; wherein the one or more longitudinal flexible embolic member is inserted into the aneurysm sac through a central opening of the neck bridge; and the neck bridge prevents the one or more longitudinal flexible members from protruding into the parent vessel of the aneurysm.

In an example, a torus can be a revolution of a circle or an ellipse in three-dimensional space. In an example, when modeling a torus or torus-section shape of a neck bridge, a convex shape (or portion of the perimeter of this shape) which is revolved to form a torus (or a section of a torus) can be a circle or an ellipse. In an example, a torus or torus-section shaped neck bridge can be created geometrically by rotating a circle or an ellipse around an axis of revolution (e.g. a vertical axis in space) which is to the right of the circle or ellipse. In an example, a torus or torus-section shaped neck bridge can be created geometrically by rotating a circle or an ellipse around an axis of revolution which is coplanar with the circle or ellipse.

In an example, when modeling a torus-section shape of a neck bridge, the lower half of a convex shape (e.g. a semi-circle) can be revolved in three-dimensional space to form a lower-half-torus (which is similar to the shape of a bundt cake mold). In an example, a torus or torus-section shaped neck bridge can be the outer surface of a torus as opposed to a solid torus or torus-section structure. In an example, when modeling a torus or torus-section shape of a neck bridge, a revolution diameter can be greater than the convex shape diameter. In an example, the revolution diameter can be at least 10% greater than the convex shape diameter. In an example, the revolution diameter can be between 10% and 25% greater than the convex shape diameter.

In an example, when modeling a neck bridge with a torus-section shape, a torus-section shape can be modeled by revolving a circle with a gap in its perimeter, wherein the circle is revolved in three-dimensional space around an axis of revolution which is coplanar with the circle, wherein clock-hour coordinates are defined around the perimeter of the circle, wherein the axis of revolution is parallel to a line between the 12 o'clock to 6 o'clock coordinates on the perimeter of the circle, wherein the axis of revolution is closest to the 9 o'clock coordinate on the perimeter of the circle, and wherein the gap is located between the 1 o'clock and 5 o'clock coordinates on the circle.

In an example, when modeling a torus or torus-section shape of a neck bridge, between 70% and 90% of the perimeter of a convex shape (e.g. a circle, ellipse, or convex polygon) can be revolved around a coplanar axis in three-dimensional space to form a section of a torus. In an example, a longitudinal axis (e.g. the longest axis) of a radially-asymmetric convex shape (e.g. an ellipse) which is revolved to form a torus can be 20% to 40% greater than a lateral axis (e.g. the shortest axis) of the convex shape. In an example, a convex shape (e.g. circle) with a 20% to 40% gap (e.g. missing section) on the side of its perimeter which faces away from the axis of revolution can be revolved in three-dimensional space to form a torus-section which is shaped like a yo-yo.

In an example, when modeling a torus or torus-section shape of a neck bridge, an ellipse can be revolved in three-dimensional space around an axis of revolution which is coplanar with the ellipse. In an example, an ellipse can be revolved in three-dimensional space around an axis of revolution which is coplanar with the ellipse, wherein the distal portion of the ellipse tilts away from the axis of revolution. In an example, an ellipse can be revolved in three-dimensional space around an axis of revolution which is coplanar with the ellipse, wherein an extension of the longitudinal axis of the ellipse intersects the axis of revolution at an angle between 20% and 50%. In an example, an ellipse can be revolved in three-dimensional space around an axis of revolution which is coplanar with the ellipse, wherein an extension of the longitudinal axis of the ellipse intersects the axis of revolution at an angle between 20% and 50%, and wherein the axis of revolution is closer to the proximal portion of the ellipse than the distal portion of the ellipse.

In an example, when modeling a torus or torus-section shape of a neck bridge, a convex shape (e.g. circle, ellipse, or convex polygon) with a gap in its perimeter can be revolved around a coplanar axis in three-dimensional space to form a section of a torus, wherein the gap is approximately one-fourth of the perimeter of the convex shape. In an example, a quarter-perimeter gap can be further specified in terms of quadrants of the convex shape (e.g. left distal quadrant, right distal quadrant, left proximal quadrant, and right proximal quadrant). In an example, a quarter-perimeter gap can be further specified in terms of clock-hour coordinates (e.g. between 9 and 12 o'clock; between 12 and 3 o'clock; between 3 and 6 o'clock; and between 6 and 9 o'clock).

In an example, when modeling a torus shape of a neck bridge, an entire convex shape (e.g. circle, ellipse, or convex polygon) is revolved around a coplanar axis in three-dimensional space to form a torus. In an example, when modeling a torus-section shape of a neck bridge, only a section of a convex shape (e.g. circle, ellipse, or convex polygon) is revolved around a coplanar axis in three-dimensional space to form a torus section. In an example, a convex shape (or portion of the perimeter of this shape) which is revolved to form a torus (or a section of a torus) can be an ellipse. In an example, a torus or torus-section shaped neck bridge can have a shape comprising an ellipsoid with a thin gap bisecting the ellipsoid.

In an example, when modeling a neck bridge with a torus-section shape, a torus-section shape can be modeled by revolving a circle with a gap in its perimeter, wherein the circle is revolved in three-dimensional space around an axis of revolution which is coplanar with the circle, wherein clock-hour coordinates are defined around the perimeter of the circle, wherein the axis of revolution is parallel to a line between the 12 o'clock to 6 o'clock coordinates on the perimeter of the circle, wherein the axis of revolution is closest to the 9 o'clock coordinate on the perimeter of the circle, wherein the gap comprises between 20% and 30% of the perimeter of the circle, and wherein the gap is located between the 1 o'clock and 5 o'clock coordinates on the circle.

In an example, when modeling a torus or torus-section shape of a neck bridge, a convex shape (e.g. circle, ellipse, or convex polygon) with a gap in its perimeter can be revolved around a coplanar axis in three-dimensional space to form a section of a torus, wherein the gap is between 10% and 25% of the perimeter of the convex shape. In an example, when modeling a torus or torus-section shape of a neck bridge, a longitudinal axis (e.g. the longest axis) of a radially-asymmetric convex shape (e.g. an ellipse) which is revolved to form a torus can be 30% to 60% greater than a lateral axis (e.g. the shortest axis) of the convex shape.

In an example, when modeling a torus or torus-section shape of a neck bridge, a revolution diameter can be between 20% to 50% greater than a convex shape diameter. In an example, a longitudinal axis (e.g. the longest axis) of a radially-asymmetric convex shape (e.g. an ellipse) which is revolved to form a torus can be twice as long as a lateral axis (e.g. the shortest axis) of the convex shape. In an example, the shape of a torus or torus-section shaped neck bridge can be modeled by revolving an ellipse in three-dimensional space around an axis of revolution which is coplanar with the ellipse, wherein the revolution diameter (defined earlier) is greater than the convex shape diameter (defined earlier), and wherein the longest axis (e.g. longitudinal axis) of the ellipse is parallel to the axis of revolution.

In an example, when modeling a neck bridge with a torus-section shape, a torus-section shape can be modeled by revolving a circle with a gap in its perimeter, wherein the circle is revolved in three-dimensional space around an axis of revolution which is coplanar with the circle, wherein clock-hour coordinates are defined around the perimeter of the circle, wherein the axis of revolution is parallel to a line between the 12 o'clock to 6 o'clock coordinates on the perimeter of the circle, wherein the axis of revolution is closest to the 9 o'clock coordinate on the perimeter of the circle, wherein the gap comprises between 5% and 15% of the perimeter of the circle, and wherein the gap is located between the 1 o'clock and 5 o'clock coordinates on the circle.

In an example, the shape of a neck bridge can be geometrically modeled by revolving a convex serpentine shape (such as a capital letter “S”) in three-dimensional space around an axis of revolution which is coplanar with the serpentine shape. This creates a complex-torus shape with two annular chambers. In an example, this creates a distal annular chamber and a proximal annular chamber, wherein these two chambers are stacked within the device. In an example, the distal annular chamber can have a concavity which opens toward the axis of rotation and the proximal annular chamber can have a concavity which opens away from the axis of rotation.

In an example, rotating the (clock-hour or compass) location of a gap around the perimeter of a convex shape which is revolved in three-dimensional space changes the location of an opening in the surface of the resulting torus shape. For example, assuming that the axis of revolution is closest to the 9 o'clock position on the convex shape, a gap between the 11 and 1 o'clock locations in the perimeter of the convex shape creates a virtually slices off a section of the distal surface of the torus shape. For example, again assuming that the axis of revolution is closest to the 9 o'clock position on the convex shape, a gap between the 2 and 4 o'clock locations in the perimeter of the convex shape creates a central gap in the outer surface of the torus shape, while leaving the central column opening intact, similar to the shape of a yo-yo toy.

In an example, when modeling a torus or torus-section shape of a neck bridge, a convex shape (e.g. circle) with a 35% to 55% gap (e.g. missing section) on the side of its perimeter which faces away from the axis of revolution can be revolved in three-dimensional space to form a torus-section which is shaped like a public-plaza water fountain (e.g. with an upper basin from which water flows out into a lower basin).

In an example, a torus or torus-section shaped neck bridge can be formed by inverting and/or everting the ends of a tubular mesh. In an example, a torus or torus-section shaped neck bridge can be formed by inverting and/or everting a tubular mesh multiple times. In an example, a complex torus shape can be formed comprising a plurality of annular chambers. In an example, a complex torus shape can be formed comprising a stack of two or more annular chambers. In an example, a complex torus shape can be formed comprising a proximal-to-distal stack of two or more annular chambers within a globular mesh. In an example, a complex torus shape with a plurality of annular chambers can be formed by inverting and/or everting the ends of tubular mesh multiple times.

In an example, when modeling a neck bridge with a torus-section shape, a torus-section shape can be modeled by revolving a circle with a gap in its perimeter, wherein the circle is revolved in three-dimensional space around an axis of revolution which is coplanar with the circle, wherein clock-hour coordinates are defined around the perimeter of the circle, wherein the axis of revolution is parallel to a line between the 12 o'clock to 6 o'clock coordinates on the perimeter of the circle, wherein the axis of revolution is closest to the 9 o'clock coordinate on the perimeter of the circle, and wherein the gap is located between the 9 o'clock and 3 o'clock coordinates on the circle.

In an example, the shape of a neck bridge can be geometrically modeled by revolving a teardrop shape in three-dimensional space around an axis of revolution which is coplanar with the teardrop shape. In an example, the one vertex of the teardrop shape can point downward and inward toward the axis of revolution. In an example, an extension of the longitudinal axis of the teardrop shape which goes through the one vertex of the teardrop shape can intersect the axis of revolution at an acute angle. In an example, an extension of the longitudinal axis of the teardrop shape which goes through the one vertex of the teardrop shape can intersect the axis of revolution at a 30 to 50 degree angle.

In an example, when modeling a neck bridge with a torus-section shape, the torus-section shape can be modeled by revolving a circle with a gap in its perimeter, wherein the circle is revolved in three-dimensional space around an axis of revolution which is coplanar with the circle, wherein clock-hour coordinates are defined around the perimeter of the circle, wherein the axis of revolution is parallel to a line between the 12 o'clock to 6 o'clock coordinates on the perimeter of the circle, wherein the axis of revolution is closest to the 9 o'clock coordinate on the perimeter of the circle, and wherein the gap is located between the 1 o'clock and 4 o'clock coordinates on the circle.

In an example, a torus or torus-section shaped neck bridge can be radially-expanded and longitudinally-compressed after insertion into an aneurysm sac. In an example, a torus or torus-section shaped neck bridge can self-expand radially and shrink longitudinally after it has been released from a catheter into an aneurysm sac. In an example, a torus or torus-section shaped neck bridge can be radially-expanded and longitudinally-compressed when an operator pulls, pushes, or rotates a wire attached to the neck bridge after the neck bridge has been inserted into an aneurysm sac. In an example, a torus or torus-section shaped neck bridge can be radially-expanded and longitudinally-compressed when an operator pulls, pushes, or rotates a wire attached to a distal portion of the neck bridge after the neck bridge has been inserted into an aneurysm sac.

In an example, when modeling a torus or torus-section shape of a neck bridge, a convex shape (e.g. circle) with a 35% to 55% gap (e.g. missing section) on the side of its perimeter which faces away from the axis of revolution can be revolved in three-dimensional space to form a torus-section which is shaped like a yo-yo.

In an example, when modeling a neck bridge with a torus-section shape, a torus-section shape can be modeled by revolving a circle with a gap in its perimeter, wherein the circle is revolved in three-dimensional space around an axis of revolution which is coplanar with the circle, wherein clock-hour coordinates are defined around the perimeter of the circle, wherein the axis of revolution is parallel to a line between the 12 o'clock to 6 o'clock coordinates on the perimeter of the circle, wherein the axis of revolution is closest to the 9 o'clock coordinate on the perimeter of the circle, wherein the gap comprises between 10% and 25% of the perimeter of the circle, and wherein the gap is located between the 1 o'clock and 5 o'clock coordinates on the circle.

In an example, the shape of a torus or torus-section shaped neck bridge can be modeled by revolving an ellipse in three-dimensional space around an axis of revolution which is coplanar with the ellipse, wherein the revolution diameter (defined earlier) is greater than the convex shape diameter (defined earlier), wherein a linear extension of the longest axis (e.g. longitudinal axis) of the ellipse intersects the axis of revolution at an acute angle, and wherein the distal end of the ellipse is farther from the axis of revolution than the proximal end of the ellipse.

In an example, when modeling a neck bridge with a torus-section shape, a torus-section shape can be modeled by revolving a circle with a gap in its perimeter, wherein the circle is revolved in three-dimensional space around an axis of revolution which is coplanar with the circle, wherein clock-hour coordinates are defined around the perimeter of the circle, wherein the axis of revolution is parallel to a line between the 12 o'clock to 6 o'clock coordinates on the perimeter of the circle, wherein the axis of revolution is closest to the 9 o'clock coordinate on the perimeter of the circle, and wherein the gap is located between the 10 o'clock and 2 o'clock coordinates on the circle.

In an example, when modeling a torus or torus-section shape of a neck bridge, a longitudinal axis (e.g. the longest dimension) of a convex shape which is revolved to form a torus can be parallel to the axis around which the convex shape is revolved. In an example, the shape of a torus or torus-section shaped neck bridge can be geometrically modeled by revolving a convex serpentine shape (such as a capital letter “S”) in three-dimensional space around an axis of revolution which is coplanar with the serpentine shape.

In an example, when modeling a neck bridge with a torus-section shape, the torus-section shape can be modeled by revolving a circle with a gap in its perimeter, wherein the circle is revolved in three-dimensional space around an axis of revolution which is coplanar with the circle, wherein clock-hour coordinates are defined around the perimeter of the circle, wherein the axis of revolution is parallel to a line between the 12 o'clock to 6 o'clock coordinates on the perimeter of the circle, wherein the axis of revolution is closest to the 9 o'clock coordinate on the perimeter of the circle, and wherein the gap is located between the 12 o'clock and 3 o'clock coordinates on the circle.

In an example, when modeling a torus or torus-section shape of a neck bridge, a convex shape (e.g. circle, ellipse, or convex polygon) with a gap in its perimeter can be revolved around a coplanar axis in three-dimensional space to form a section of a torus, wherein the gap is between 20% and 40% of the perimeter of the convex shape. In an example, a longitudinal axis (e.g. the longest axis) of a radially-asymmetric convex shape (e.g. an ellipse) which is revolved to form a torus can be 10% to 25% greater than a lateral axis (e.g. the shortest axis) of the convex shape. In an example, a torus or torus-section neck bridge can have a chalice and/or wineglass shaped cross-sectional perimeter.

In an example, when modeling a torus or torus-section shape of a neck bridge, half of the perimeter of a convex shape (e.g. circle, ellipse, or convex polygon) can be revolved around a coplanar axis in three-dimensional space to form a section of a torus. In an example, a convex shape (e.g. circle, ellipse, or convex polygon) with a gap in its perimeter can be revolved around a coplanar axis in three-dimensional space to form a section of a torus. In an example, a section (e.g. percentage) of the perimeter of a convex shape can be revolved in three-dimensional space to form a section of a torus.

In an example, the shape of a neck bridge can be geometrically modeled by revolving a convex serpentine shape (such as a capital letter “S”) in three-dimensional space around an axis of revolution which is coplanar with the serpentine shape. This creates a complex-torus shape with two annular chambers. In an example, this creates a distal annular chamber and a proximal annular chamber, wherein these two chambers are stacked within the device. In an example, the distal annular chamber can have a concavity which away from the axis of rotation and the proximal annular chamber can have a concavity which opens toward the axis of rotation.

In an example, when modeling a torus or torus-section shape of a neck bridge, a longitudinal axis (e.g. the longest dimension) of a convex shape which is revolved to form a torus can be perpendicular to the axis around which the convex shape is revolved. In an example, a section (e.g. percentage) of a convex shape (e.g. circle, ellipse, or convex polygon) can be revolved around a coplanar axis in three-dimensional space to form a section of a torus. In an example, a convex shape (e.g. circle, ellipse, or convex polygon) with a gap in its perimeter can be revolved around a coplanar axis in three-dimensional space to form a section of a torus, wherein the gap is approximately one-third of the perimeter of the convex shape.

In an example, when modeling a torus or torus-section shape of a neck bridge, a revolution diameter can be 15% to 35% greater than a convex shape diameter. In an example, a torus-section shaped neck bridge can have a stylized “eaten apple” shape, wherein the central proximal and central distal surfaces of the neck bridge are that same as that of a complete torus, but there is a central circumferential gap comprising between 15% to 35% of the perimeter. In an example, this “eaten apple” shape can be formed by revolving a circle with a gap between the 2 and 4 o'clock coordinates.

In an example, when modeling a torus or torus-section shape of a neck bridge, a section (e.g. percentage) of the perimeter of a convex shape (e.g. circle, ellipse, or convex polygon) can be revolved around a coplanar axis in three-dimensional space to form a section of a torus. In an example, the shape of a torus or torus-section shaped neck bridge can be modeled by revolving an ellipse in three-dimensional space around an axis of revolution which is coplanar with the ellipse, wherein the revolution diameter (defined earlier) is greater than the convex shape diameter (defined earlier). In an example, a revolution diameter can be 40% to 60% greater than a convex shape diameter.

In an example, a torus or torus section shape neck bridge can have a shape which is modeled by revolving an oval in three-dimensional space around an axis of revolution. In an example, a torus or torus section shape neck bridge can have a shape which is modeled by revolving an oval in three-dimensional space around an axis of revolution, wherein the axis of revolution is parallel to the longitudinal axis of the oval. In an example, a torus or torus section shape neck bridge can have a shape which is modeled by revolving an oval in three-dimensional space around an axis of revolution, wherein the revolution diameter is between 5% and 20% greater than the convex shape diameter (e.g. the lateral width of the oval).

In an example, when modeling a neck bridge with a torus-section shape, the torus-section shape can be modeled by revolving a circle with a gap in its perimeter, wherein the circle is revolved in three-dimensional space around an axis of revolution which is coplanar with the circle, wherein clock-hour coordinates are defined around the perimeter of the circle, wherein the axis of revolution is parallel to a line between the 12 o'clock to 6 o'clock coordinates on the perimeter of the circle, wherein the axis of revolution is closest to the 9 o'clock coordinate on the perimeter of the circle, and wherein the gap is located between the 2 o'clock and 4 o'clock coordinates on the circle.

In an example, an intrasaccular aneurysm occlusion device can further comprise a pusher wire which pushes the neck bridge through a catheter. In an example, an intrasaccular aneurysm occlusion device can further comprise: a pusher wire which pushes the neck bridge through a catheter; and a detachment mechanism which connects the pusher wire to the neck bridge, wherein the detachment mechanism can be remotely activated by the device operator to disconnect the neck bridge from the pusher wire. In an example, the detachment mechanism can be melted by the application of electrical energy sent from the device operator. In an example, an intrasaccular aneurysm occlusion device can further comprise an electrolytic detachment mechanism.

In an example, the shape of a torus or torus-section shaped neck bridge can be modeled by revolving an ellipse in three-dimensional space around an axis of revolution which is coplanar with the ellipse, wherein the revolution diameter (defined earlier) is greater than the convex shape diameter (defined earlier), and wherein a linear extension of the longest axis (e.g. longitudinal axis) of the ellipse intersects the axis of revolution at an acute angle in the range of 20 to 45 degrees. In an example, when modeling a torus or torus-section shape of a neck bridge, a revolution diameter can be at least 25% greater than a convex shape diameter. In an example, when modeling a torus or torus-section shape of a neck bridge, between 55% and 70% of the perimeter of a convex shape (e.g. circle, ellipse, or convex polygon) can be revolved around a coplanar axis in three-dimensional space to form a section of a torus.

In an example, the shape of a torus or torus-section shaped neck bridge can be modeled by revolving an ellipse in three-dimensional space around an axis of revolution which is coplanar with the ellipse, wherein the revolution diameter (defined earlier) is greater than the convex shape diameter (defined earlier), and wherein the longest axis (e.g. longitudinal axis) of the ellipse is perpendicular to the axis of revolution. In an example, when modeling a torus or torus-section shape of a neck bridge, a convex shape (e.g. circle, ellipse, or convex polygon) with a gap in its perimeter can be revolved around a coplanar axis in three-dimensional space to form a section of a torus, wherein the gap is half of the perimeter of the convex shape. In an example, a revolution diameter can be 5% to 20% greater than a convex shape diameter.

In an example, when modeling a torus or torus-section shape of a neck bridge, a convex shape (e.g. circle, ellipse, or convex polygon) with a gap in its perimeter can be revolved around a coplanar axis in three-dimensional space to form a section of a torus, wherein the gap is between 30% and 60% of the perimeter of the convex shape. In an example, when modeling a torus or torus-section shape of a neck bridge, a convex shape (e.g. circle) with a 20% to 40% gap (e.g. missing section) on the side of its perimeter which faces away from the axis of revolution can be revolved in three-dimensional space to form a torus-section which is shaped like a public-plaza water fountain.

In an example, the shape of a torus or torus-section shaped neck bridge can be modeled by revolving an ellipse in three-dimensional space around an axis of revolution which is coplanar with the ellipse, wherein the revolution diameter (defined earlier) is greater than the convex shape diameter (defined earlier), and wherein a linear extension of the longest axis (e.g. longitudinal axis) of the ellipse intersects the axis of revolution at an acute angle. In an example, when modeling a torus or torus-section shape of a neck bridge, three-quarters of the perimeter of a convex shape (e.g. circle, ellipse, or convex polygon) can be revolved around a coplanar axis in three-dimensional space to form a section of a torus.

In an example, when modeling a neck bridge with a torus-section shape, the torus-section shape can be modeled by revolving a circle with a gap in its perimeter, wherein the circle is revolved in three-dimensional space around an axis of revolution which is coplanar with the circle, wherein clock-hour coordinates are defined around the perimeter of the circle, wherein the axis of revolution is parallel to a line between the 12 o'clock to 6 o'clock coordinates on the perimeter of the circle, wherein the axis of revolution is closest to the 9 o'clock coordinate on the perimeter of the circle, and wherein the gap is located between the 12 o'clock and 6 o'clock coordinates on the circle.

In an example, a torus or torus section shaped neck bridge can have a shape which can be modeled by revolving a teardrop shape in three-dimensional space around an axis of revolution which is coplanar with the teardrop shape. In an example, a torus or torus section shaped neck bridge can have a shape which can be modeled by revolving a teardrop shape in three-dimensional space around an axis of revolution which is coplanar with the teardrop shape, wherein an extension of the longitudinal axis of the teardrop shape intersects the axis of revolution at an acute angle, wherein a distal surface of the teardrop shape is farther from the axis of revolution than a proximal vertex of the teardrop shape, and wherein the revolution diameter is greater than the teardrop shape diameter (e.g. defined earlier as the convex shape diameter).

In an example, a central opening of a torus or torus section shaped neck bridge can comprise a central column or cylinder which spans from a proximal surface of the neck bridge to a distal surface of the neck bridge. In an example, this column can have tapered, funnel-shaped, double-funnel-shaped and/or hyperboloidal. In an example, a torus or torus section shaped neck bridge wherein the revolution diameter is greater than the convex shape diameter automatically forms a columnar opening through the longitudinal (e.g. proximal to distal) center of the neck bridge. In an example, a torus or torus section shaped neck bridge wherein the revolution diameter is greater than the convex shape diameter automatically forms a columnar opening through the longitudinal (e.g. proximal to distal) center of the neck bridge, wherein embolic members and/or material can be inserted into the aneurysm sac through the columnar opening.

In an example, a torus or torus section shaped neck bridge can have a shape which can be modeled by revolving an egg shape in three-dimensional space around an axis of revolution which is coplanar with the egg shape. In an example, a torus or torus section shaped neck bridge can have a shape which can be modeled by revolving an egg shape in three-dimensional space around an axis of revolution which is coplanar with the egg shape, wherein an extension of the longitudinal axis of the egg shape intersects the axis of revolution at an acute angle, wherein a distal surface of the egg shape is farther from the axis of revolution than a proximal surface of the egg shape, and wherein the revolution diameter is greater than the egg shape diameter (e.g. defined earlier as the convex shape diameter).

In an example, the shape of a torus or torus-section shaped neck bridge can be modeled by revolving a convex shape in three-dimensional space around an axis of revolution which is coplanar with the convex shape, wherein the convex shape is a conic-section shape. In an example, the shape of a torus or torus-section shaped neck bridge can be modeled by revolving a convex shape in three-dimensional space around an axis of revolution which is coplanar with the convex shape, wherein the convex shape is an egg shape.

In an example, a torus-section shaped neck bridge can have a stylized “eaten apple” shape, wherein the central proximal and central distal surfaces of the neck bridge are that same as that of a complete torus, but there is a central circumferential gap comprising between 20% and 40% of the perimeter. In an example, this “eaten apple” shape can be formed by revolving a circle with a gap between the 1 and 5 o'clock coordinates. In an example, the shape of a torus or torus-section shaped neck bridge can be modeled by revolving a convex shape in three-dimensional space around an axis of revolution which is coplanar with the convex shape, wherein the convex shape is a football shape.

In an example, a torus or torus-section shaped neck bridge can be the lower surface of the lower half of a torus. In an example, a torus or torus-section shaped neck bridge can be a circular stent. In an example, a torus or torus-section shaped neck bridge can be a cylindrical stent. In an example, a torus or torus-section shaped neck bridge can be a toroidal stent. In an example, the shape of a torus or torus-section shaped neck bridge can be modeled by revolving a convex shape in three-dimensional space around an axis of revolution which is coplanar with the convex shape, wherein the convex shape is an oval shape. In an example, a torus or torus-section shaped neck bridge can be a tubular stent.

In an example, a “yin and yang” symbol can be modeled geometrically by: (a) using a diameter dividing line to divide a circle with radius x into two half-circle sections; (b) adding a half-circle area with radius one-half x (on a one half of the dividing line) to the first section (and subtracting this area from the second section); and (c) adding a half-circle area with radius one-half x (on the other half of the dividing line) to the second section (and subtracting this area of the first section). Alternatively, a “yin and yang” symbol can be modeled as two interlocking apostrophes with reflected vertical orientations and reflected horizontal orientations. Interior circles with contrasting colors are often added to each of the “yin” and “yang” sections in religious iconography, but are not included in the general shape referenced here.

In an example, a neck bridge can have a compound torus shape which is modeled by revolving a circular “yin and yang” symbol (without an interior circle or dot in each “yin” or “yang” section). In an example, a neck bridge can have a compound torus shape which is modeled by revolving a circular “yin and yang” symbol in three-dimensional space around an axis of revolution which is coplanar with the circular “yin and yang” symbol. In another example, a neck bridge can have a torus shape which is modeled by revolving one section (e.g. “yin” or “yang”) selected from a “yin and yang” symbol. In another example, a neck bridge can have a torus shape which is modeled by revolving one section (e.g. “yin” or “yang”) selected from a “yin and yang” symbol in three-dimensional space around an axis of revolution which is coplanar with the section.

In an example, a torus or torus-section shaped neck bridge can be an ellipsoidal stent. In an example, a torus or torus-section shaped neck bridge can be a toroidal stent. In an example, when modeling a torus or torus-section shape of a neck bridge, a convex shape (e.g. circle) with a gap (e.g. missing section) on the side of its perimeter which faces away from the axis of revolution can be revolved in three-dimensional space to form a torus-section which is shaped like a yo-yo. In an example, the shape of a torus or torus-section shaped neck bridge can be modeled by revolving a convex shape in three-dimensional space around an axis of revolution which is coplanar with the convex shape, wherein the convex shape is an oblate circle. In an example, when modeling a torus or torus-section shape of a neck bridge, a convex shape (e.g. circle) with a side gap on its perimeter can be revolved in three-dimensional space to form a torus-section shape.

In an example, when modeling a torus or torus-section shape of a neck bridge, a convex shape (or portion of the perimeter of this shape) which is revolved to form a torus (or a section of a torus) can have an egg shape. In an example, the shape of a torus or torus-section shaped neck bridge can be modeled by revolving a convex shape in three-dimensional space around an axis of revolution which is coplanar with the convex shape, wherein the convex shape is a convex lens shape. In an example, a convex shape (or portion of the perimeter of this shape) which is revolved to form a torus (or a section of a torus) can be a convex polygon. In an example, the shape of a torus or torus-section shaped neck bridge can be modeled by revolving a convex shape in three-dimensional space around an axis of revolution which is coplanar with the convex shape, wherein the convex shape is a tear-drop shape.

In an example, when modeling a torus or torus-section shape of a neck bridge, a convex shape (e.g. circle) with a gap (e.g. missing section) on the side of its perimeter which faces away from the axis of revolution can be revolved in three-dimensional space to form a torus-section which is shaped like a public-plaza water fountain. In an example, when modeling a torus or torus-section shape of a neck bridge, a convex shape (or portion of the perimeter of this shape) which is revolved to form a torus (or a section of a torus) can be an equilateral hexagon. In an example, a torus or torus-section shaped neck bridge can have the shape of the lower surface of the lower half of a torus.

In an example, a torus or torus-section shaped neck bridge can comprise two meshes and a liquid-impermeable layer between the two meshes. In an example, a torus or torus-section shaped neck bridge can have two or more layers. In an example, a torus or torus-section shaped neck bridge can further comprise a reinforcing ring around the proximal-to-distal longitudinal center of a central double-funnel, hourglass, and/or hyperbolic shaped opening. In an example, there can be a reinforcing ring (e.g. thicker wire or band) at the distal end of a central opening in a torus or torus-section shaped neck bridge. In an example, there can be a reinforcing ring (e.g. thicker wire or band) in the middle of a central opening in a torus or torus-section shaped neck bridge.

In an example, a torus or torus-section shaped neck bridge can comprise wires or filaments made from superelastic material. In an example, a torus or torus-section shaped neck bridge can be an expandable wire frame. In an example, the neck bridge can comprise: a plurality of circumferential members (e.g. wires, strands, or filaments) which are centered on the axis of revolution of the torus or torus section; and a plurality of concave members (e.g. wires, strands, or filaments) which extend from the proximal surface of the neck bridge to the distal surface of the neck bridge, wherein the circumferential members and the concave members are woven or braided together. In an example, a torus or torus-section shaped neck bridge can comprise a plurality of braided or woven filaments. In an example, a torus or torus-section shaped neck bridge can comprise a honeycomb mesh (e.g. mesh with hexagonal openings).

In an example, there can be two nested (e.g. coaxial and/or concentric) cylinders, tubes, rings, and/or bands which are centered around the longitudinal axis of an opening through a torus or torus-section shape neck bridge. In an example, the mesh of the neck bridge can be held (e.g. pinched, compressed, welded, or glued) between the two nested (e.g. coaxial and/or concentric) cylinders, tubes, rings, and/or bands and embolic members or material can be inserted through the center of the inner cylinder, tube, ring, and/or band.

In an example, there can be an inner cylinder, tube, ring, and/or band centered around the longitudinal axis of an opening through a torus or torus-section shape neck bridge and an outer cylinder, tube, ring, and/or band which is also centered around this axis. In an example, the inner and outer cylinders, tubes, rings, and/or bands can be nested, coaxial, and/or concentric. In an example, the mesh of the neck bridge can be held (e.g. pinched, compressed, welded, or glued) between the inner and outer cylinders, tubes, rings, and/or bands. In an example, embolic members or material can be inserted through the center of the inner cylinder, tube, ring, and/or band.

In an example, a torus or torus-section shaped neck bridge can further comprise radio-opaque sections or markers. In an example, a torus or torus-section shaped neck bridge can comprise a mesh, net, braid, or stent. In an example, a torus or torus-section shaped neck bridge can further comprise a reinforcing ring (e.g. relatively-thick wires) around widest portion of the perimeter of the neck bridge. In an example, a torus or torus-section shaped neck bridge can further comprise one or more reinforcing rings (e.g. thicker wires or bands) around the widest circumference of the neck bridge. In an example, a torus or torus-section shaped neck bridge can further comprise a reinforcing ring around a central double-funnel, hourglass, and/or hyperbolic shaped opening.

In an example, a torus or torus-section shaped neck bridge can have two layers. In an example, a torus or torus-section shaped neck bridge can have a single layer. In an example, a torus or torus-section shaped neck bridge can comprise a plurality of braided wire or filaments. In an example, a torus or torus-section shaped neck bridge can comprise a wire mesh, net, braid, or stent. In an example, a proximal portion of torus or torus-section shaped neck bridge can have two layers and a distal portion of the neck bridge can have one layer. In an example, the neck bridge can comprise a honeycomb mesh (e.g. mesh with hexagonal openings).

In an example, a torus or torus-section shaped neck bridge can be a woven or braided wire mesh and/or frame. In an example, the neck bridge can comprise: at least one helical member (e.g. wire, strand, or filament) which is centered on the axis of revolution of the torus or torus section; and a plurality of radial members (e.g. wires, strands, or filaments) along radial vectors which extend outward from the axis of revolution. In an example, a torus or torus-section shaped neck bridge can comprise wires or filaments made from shape memory material. In an example, a torus or torus-section shaped neck bridge can be a wire mesh and/or frame.

In an example, the neck bridge can comprise a plurality of radial struts. In an example, a plurality of radial struts can be embedded into a torus or torus-section shaped neck bridge. In an example, the neck bridge can comprise a plurality of circumferential rings. In an example, a plurality of circumferential rings can be embedded into a torus or torus-section shaped neck bridge. In an example, the neck bridge can comprise a plurality of wire loops. In an example, a plurality of wire loops can be embedded into a torus or torus-section shaped neck bridge.

In an example, the neck bridge can comprise: at least one helical member (e.g. wire, strand, or filament) which is centered on the axis of revolution of the torus or torus section; and a plurality of radial members (e.g. wires, strands, or filaments) along radial vectors which extend outward from the axis of revolution, wherein the helical member and the radial members are woven or braided together. In an example, a torus or torus-section shaped neck bridge can comprise a mesh, network, lattice, or radial array of wires or other stiff fibers. In an example, a torus or torus-section shaped neck bridge can have a single layer. In an example, a torus or torus section shaped neck bridge can comprise: a plurality of rings which are centered around the axis of revolution; and a plurality of rings which are centered on the path of the center of the revolved convex shape as it is revolved in three-dimensional space.

In an example, the neck bridge can comprise: a plurality of circumferential members (e.g. wires, strands, or filaments) which are centered on the axis of revolution of the torus or torus section; and a plurality of concave members (e.g. wires, strands, or filaments) along radial vectors which extend from the proximal surface of the neck bridge to the distal surface of the neck bridge. In an example, a torus or torus-section shaped neck bridge can further comprise a reinforcing ring around the proximal end of a central double-funnel, hourglass, and/or hyperbolic shaped opening. In an example, a torus or torus-section shaped neck bridge can be braided or woven from wires or drawn tubes. In an example, a torus or torus-section shaped neck bridge can further comprise a reinforcing ring around the distal end of a central double-funnel, hourglass, and/or hyperbolic shaped opening.

In an example, a torus or torus-section shaped neck bridge can be made by welding, gluing, or crimping the ends of a tubular mesh to each other. In an example, a torus-shaped neck bridge can be made by inverting the ends of a tubular mesh into the tube and then connecting the ends of the tubular mesh to each other within the tube. In an example, a torus or torus-section shaped neck bridge can be made from a combination of metal components (e.g. wires) and polymer components (e.g. yarns or fibers). In an example, the outer perimeter of a torus or torus-section shaped neck bridge can be more elastic than the central portion of the neck bridge.

In an example, a torus or torus-section shaped neck bridge can be braided or woven from metal wires or micro-tubes. In an example, a torus or torus-section shaped neck bridge can be braided or woven from drawn filled tubing wires. In an example, the outer perimeter of a torus or torus-section shaped neck bridge can have lower porosity than the central portion of the neck bridge. In an example, a torus or torus-section shaped neck bridge can be made by curving a tubular mesh into a circle and then attaching the ends of the tubular mesh to each other.

In an example, portions of a neck bridge can be braided from wire and portions can be made from a polymer. In an example, a method for making a torus or torus-section shaped neck bridge can comprise inverting the ends of a tubular mesh into the mesh and then connecting ends of the mesh together by adhesion, welding, crimping, braiding, or tying. In an example, a first annular member (e.g. ring or band) can be attached to a first end of a tubular mesh and a second annular member (e.g. ring or band (can be attached to a second end of the tubular mesh, wherein a torus-shaped neck bridge is formed by connecting the first annular member and the second annular member together. In an example, this connection can occur before the neck bridge is inserted into a catheter for delivery to an aneurysm sac. In an example, this connection can occur after tubular mesh has been inserted into an aneurysm sac.

In an example, a torus or torus-section shaped neck bridge can comprise a wire frame and a polymer layer. In an example, a torus or torus-section shaped neck bridge can be 3D printed from a polymer. In an example, portions of a neck bridge can be made from wire and portions can be made from a polymer. In an example, a torus or torus-section shaped neck bridge can be made from a polymer. In an example, the outer perimeter of a torus or torus-section shaped neck bridge can be less elastic than the central portion of the neck bridge. In an example, a torus or torus-section shaped neck bridge can be made from nitinol. In an example, the outer perimeter of a torus or torus-section shaped neck bridge can be more elastic than the central portion of the neck bridge. In an example, a torus or torus-section shaped neck bridge can be a woven or braided wire mesh and/or frame.

In an example, a torus or torus-section shaped neck bridge can comprise a plurality of overlapping wire or filament loops. In an example, a torus or torus-section shaped neck bridge can comprise a plurality of overlapping wire or filament loops, wherein the ends of the wires forming the loops are toward the center of the neck bridge. In an example, a torus or torus-section shaped neck bridge can comprise a plurality of overlapping wire or filament loops which extend radially-outward from the center (e.g. the central funnel-shaped opening) of the neck bridge.

In an example, a torus or torus-section shaped neck bridge can have a uniform durometer level. In an example, a torus or torus-section shaped neck bridge can be made from one or more metals and a flexible mesh can be made from one or more polymers. In an example, the outer perimeter of a torus or torus-section shaped neck bridge can have a greater durometer level than the central portion of a torus or torus-section shaped neck bridge. In an example, a torus or torus-section shaped neck bridge can be cut from metal. In an example, the outer perimeter of a torus or torus-section shaped neck bridge can have a lower durometer level than the central portion of a torus or torus-section shaped neck bridge. In an example, a torus or torus-section shaped neck bridge can be made by laser-cutting metal.

In an example, a torus or torus-section neck bridge can further comprise an annular member inside the funnel of a funnel-shaped central proximal opening, wherein this annular member serves as detachable connection mechanism which can be used by a device operator to selectively connect a neck bridge to a delivery catheter or disconnect the neck bridge from the delivery catheter. In an example, a torus or torus-section neck bridge can further comprise an annular member on the proximal half of a central proximal opening of the torus shape, wherein this annular member serves as detachable connection mechanism which can be used by a device operator to selectively connects a neck bridge to a delivery catheter or disconnect the neck bridge from the delivery catheter.

In an example, the outer perimeter of a torus or torus-section shaped neck bridge can have a greater durometer level than the central portion of the neck bridge. In an example, a torus or torus-section shaped neck bridge can comprise a 3D-printed mesh, net, braid, or stent. In an example, the outer perimeter of a torus or torus-section shaped neck bridge can have a lower durometer level than the central portion of the neck bridge. In an example, a torus or torus-section shaped neck bridge can be made from metal and polymer components. In an example, the outer perimeter of a torus or torus-section shaped neck bridge can have a greater durometer level than the central portion of the neck bridge.

In an example, a torus or torus-section shaped neck bridge can be cut, braided, or 3D printed. In an example, a torus or torus-section shaped neck bridge can have uniform porosity. In an example, a torus or torus-section shaped neck bridge can be made by connecting the ends of a tubular mesh to each other. In an example, a torus-shaped neck bridge can be made by curving a tubular mesh so that its longitudinal axis becomes circular and then connecting the ends of the tubular mesh to each other. In an example, a method for making a torus or torus-section shaped neck bridge can comprise: attaching a first part of a locking mechanism to a first end of a tubular mesh; attaching a second part of the locking mechanism to a second end of the tubular mesh; inverting the first and second ends of the tubular mesh into the mesh; and connecting the first and second parts of the locking mechanism to each other.

In an example, a torus or torus-section shaped neck bridge can comprise a wire frame and a polymer mesh. In an example, the outer perimeter of a torus or torus-section shaped neck bridge can have greater porosity than the central portion of the neck bridge. In an example, a method for making a torus or torus-section shaped neck bridge can comprise curving the tubular mesh so that its longitudinal axis becomes circular and then connecting the ends of the mesh together by adhesion, welding, crimping, braiding, or tying. In an example, a torus or torus-section shaped neck bridge can comprise braided wire and at least one polymer layer.

In an example, a torus or torus-section shaped neck bridge can be 3D printed. In an example, a tubular mesh from which a torus or torus-section shaped neck bridge is made can be tapered. In an example, a neck bridge can be made with a nitinol (NiTi) alloy. In an example, a torus or torus-section shaped neck bridge can comprise two braided wire layers and a polymer layer between the two wire layers. In an example, a torus or torus-section shaped neck bridge can a made by laser cutting. In an example, a torus or torus-section shaped neck bridge can have uniform elasticity. In an example, a torus or torus-section shaped neck bridge can be a metal stent or a polymer stent. In an example, the outer perimeter of a torus or torus-section shaped neck bridge can be less elastic than the central portion of the neck bridge.

In an example, a method for making a torus or torus-section shaped neck bridge can comprise: attaching a first part of a locking mechanism to a first end of a tubular mesh; attaching a second part of the locking mechanism to a second end of the tubular mesh; curving the tubular mesh so that its longitudinal axis becomes circular; and connecting the first and second parts of the locking mechanism to each other. In an example, a method for making a torus or torus-section shaped neck bridge can comprise: attaching a first part of an annular locking mechanism to a first end of a tubular mesh; attaching a second part of the annular locking mechanism to a second end of the tubular mesh; curving the tubular mesh so that its longitudinal axis becomes circular; and connecting the first and second parts of the annular locking mechanism to each other.

In an example, a torus or torus-section shaped neck bridge can comprise a wire frame and a polymer mesh. In an example, the outer perimeter of a torus or torus-section shaped neck bridge can have lower porosity than the central portion of the neck bridge. In an example, a torus or torus-section shaped neck bridge can be braided or woven from metal wires. In an example, the outer perimeter of a torus or torus-section shaped neck bridge can have greater porosity than the central portion of the neck bridge. In an example, a neck bridge can be made with platinum, gold, silver, cobalt, chromium, or titanium. In an example, a torus or torus-section shaped neck bridge can have a uniform durometer level. In an example, a torus or torus-section shaped neck bridge can be made from metal. In an example, the outer perimeter of a torus or torus-section shaped neck bridge can have a lower durometer level than the central portion of the neck bridge.

In an example, a torus or torus-section shaped neck bridge can be radially-compressed and longitudinally-elongated for insertion into a catheter for delivery through a person's vasculature and insertion into an aneurysm sac. In an example, a torus or torus-section shaped neck bridge can radially expand within the aneurysm sac to a width which is greater than the width of the aneurysm neck. In an example, a torus or torus-section shaped neck bridge can self-expand in a radial manner within the aneurysm sac. In an example, a torus or torus-section shaped neck bridge can self-expand because it comprises shape memory material. In an example, a torus or torus-section shaped neck bridge can self-expand radially and contract longitudinally after it has been inserted into an aneurysm sac and exits a catheter. In an example, a torus or torus-section shaped neck bridge can self-expand (e.g. expand radially) in an aneurysm sac after being released from a delivery catheter.

In an example, a torus or torus-section shaped neck bridge can further comprise a closure mechanism (e.g. a valve) which enables closure of an opening through the neck bridge after embolic members and/or material has been inserted through the opening into the aneurysm sac, wherein this closure mechanism can be remotely operated (e.g. remotely closed) by a person pulling, pushing, or rotating a wire attached to the closure mechanism. In an example, an active valve can be remotely opened and/or closed by an operator by the application of electromagnetic energy. In an example, a torus or torus-section shaped neck bridge can have a central double-funnel, hourglass, and/or hyperbolic shaped opening through which string-of-pearls embolic strands are inserted into the aneurysm sac.

In an example, a torus or torus-section shaped neck bridge can have a central double-funnel, hourglass, and/or hyperbolic shaped opening through which congealing liquid or gel is inserted into the aneurysm sac. In an example, this device can further comprise a valve in the central opening of a torus or torus-section shaped neck bridge through which one or more embolic coils are inserted. In an example, a torus or torus-section shaped neck bridge can further comprise a closure mechanism (e.g. a valve) which enables closure of an opening through the neck bridge after embolic members and/or material has been inserted through the opening into the aneurysm sac, wherein this closure mechanism is a leaflet valve.

In an example, an active valve can be remotely opened and/or closed by an operator by pulling a filament. In an example, a cross-sectional area of a valve can be between 5% to 30% of the maximum cross-sectional area of the torus or torus-section shaped neck bridge. In an example, a valve in a central opening can be a bi-leaflet valve or tri-leaflet valve, analogous to a heart valve. In an example, a torus or torus-section shaped neck bridge can further comprise a closure mechanism (e.g. a valve) which enables closure of an opening through the neck bridge after embolic members and/or material has been inserted through the opening into the aneurysm sac, wherein this closure mechanism is a tri-leaflet valve.

In an example, an active valve can be remotely opened and/or closed by an operator by cutting, pulling, or pushing a flap or plug. In an example, a torus or torus-section shaped neck bridge can further comprise a closure mechanism (e.g. a valve) which enables closure of an opening through the neck bridge after embolic members and/or material has been inserted through the opening into the aneurysm sac, wherein this closure mechanism is a one-way valve. In an example, an active valve in an opening in the neck bridge can be remotely opened and/or closed by device operator. In an example, a torus or torus-section shaped neck bridge can further comprise a closure mechanism (e.g. a valve) which enables closure of an opening through the neck bridge after embolic members and/or material has been inserted through the opening into the aneurysm sac, wherein this closure mechanism is on the distal end of the opening.

In an example, an intrasaccular aneurysm occlusion device can comprise: a torus or torus-section shaped neck bridge which is configured to be inserted into an aneurysm sac and expand to cover the neck of the aneurysm; a catheter through which the neck bridge is delivered to the aneurysm sac; and a decoupling mechanism which connects the neck bridge to the catheter, wherein the decoupling mechanism is located in a centra funnel or hyperboloidal opening in the neck bridge, and wherein the decoupling mechanism can be remotely activated (e.g. to decouple the neck bridge and the catheter) by the device operator.

In an example, a torus or torus-section shaped neck bridge can further comprise two coaxial and/or concentric rings (e.g. inner and outer rings) around the central double-funnel, hourglass, and/or hyperbolic shaped opening, wherein ends of wires comprising the neck bridge are held (e.g. pinched or adhered) between the two rings (e.g. between the inner and outer rings) and wherein embolic member and/or material is inserted through the inner ring into the aneurysm sac. In an example, a valve through an opening in a neck bridge can be a leaflet valve. In an example, a torus or torus-section shaped neck bridge can further comprise a closure mechanism (e.g. a valve) which enables closure of an funnel-shaped or hyperboloidal opening through the neck bridge after embolic members and/or material has been inserted through the opening into the aneurysm sac, wherein this closure mechanism is on the distal end of this opening. In an example, an opening through a neck bridge can have a hyperbolic cross-section.

In an example, a torus or torus-section shaped neck bridge can further comprise a closure mechanism (e.g. a valve) which enables closure of an funnel-shaped or hyperboloidal opening through the neck bridge after embolic members and/or material has been inserted through the opening into the aneurysm sac, wherein this closure mechanism is in the longitudinal (e.g. proximal-to-distal) center of this opening. In an example, embolic members or material can be inserted into an intrasaccular arcuate expandable member through (a one-way valve in) the central opening of a torus or torus-section shaped neck bridge. In an example, a torus or torus-section shaped neck bridge can further comprise a closure mechanism (e.g. a valve) which enables closure of an funnel-shaped or hyperboloidal opening through the neck bridge after embolic members and/or material has been inserted through the opening into the aneurysm sac, wherein this closure mechanism is on the proximal end of this opening.

In an example, the cross-sectional area of the central opening in the torus or torus-section shaped neck bridge can be between 5% to 30% of the maximum cross-sectional area of the torus or torus-section shaped neck bridge. In an example, a torus or torus-section shaped neck bridge can have a central opening through which embolic members and/or material (e.g. coils, beads, string-of-pearls embolic strands, microsponges, hydrogels, or congealing liquid/gel) are inserted into the aneurysm sac. In an example, a torus or torus-section shaped neck bridge can have a central double-funnel, hourglass, and/or hyperbolic shaped opening through which embolic members and/or material (e.g. coils, beads, string-of-pearls embolic strands, microsponges, hydrogels, or congealing liquid/gel) are inserted into the aneurysm sac.

In an example, a torus or torus-section shaped neck bridge can further comprise a closure mechanism (e.g. a valve) which enables closure of an opening through the neck bridge after embolic members and/or material has been inserted through the opening into the aneurysm sac. In an example, a valve through an opening in a neck bridge can be a bi-leaflet valve or tri-leaflet valve, analogous to a heart valve. In an example, a torus or torus-section shaped neck bridge can further comprise a closure mechanism (e.g. a valve) which enables closure of an opening through the neck bridge after embolic members and/or material has been inserted through the opening into the aneurysm sac, wherein this closure mechanism can be remotely operated (e.g. remotely closed) by a person implanting the device.

In an example, a valve through an opening in a neck bridge can passively open when embolic members or material is pushed through it and can passively close when insertion of the embolic members or material has been completed. An advantage of a torus (or torus section) shape for a neck bridge is that the double-funnel, hourglass, and/or hyperbolic shaped opening which is formed at the center of the torus (or torus section) can guide insertion of a catheter through the neck bridge to facilitate insertion of embolic members and/or material into the aneurysm sac. In an example, a torus or torus-section shaped neck bridge can have a central double-funnel, hourglass, and/or hyperbolic shaped opening through which embolic coils are inserted into the aneurysm sac.

In an example, a cross-sectional area of a valve can be between 5% to 15% of the maximum cross-sectional area of the torus or torus-section shaped neck bridge. In an example, a valve in a central opening can be a leaflet valve. In an example, a torus or torus-section shaped neck bridge can further comprise a closure mechanism (e.g. a valve) which enables closure of an opening through the neck bridge after embolic members and/or material has been inserted through the opening into the aneurysm sac, wherein this closure mechanism can be remotely operated (e.g. remotely closed) by a person delivering electrical energy to the closure mechanism.

In an example, an active valve can be remotely opened and/or closed by an operator by pushing, pulling, or rotating a wire. In an example, a torus (or torus section) shaped neck bridge can have advantages over a hemispherical, bowl-shaped, or flower-shaped neck bridge. In an example, a valve in a neck bridge can allow embolic coils to be inserted into the aneurysm after the neck bridge has been expanded in the aneurysm, but can close to reduce blood flow into the aneurysm after coil insertion is complete. In an example, a torus or torus-section shaped neck bridge can further comprise two coaxial and/or concentric rings around the central double-funnel, hourglass, and/or hyperbolic shaped opening.

In an example, a valve through an opening in a neck bridge can be in a hyperbolic opening through the neck bridge. In an example, a torus or torus-section shaped neck bridge can have a central double-funnel, hourglass, and/or hyperbolic shaped opening through which beads or microsponges are inserted into the aneurysm sac. In an example, a central portion of a torus or torus-section shaped neck bridge can comprise an upward-rising cone, analogous to the cone of a volcano, with the opening being where the crater of a volcano would be. In an example, a valve allows an embolic member to be inserted into an aneurysm after the neck bridge has been expanded in the aneurysm, but the valve closes to reduce blood flow into the aneurysm after the embolic member has passed through the valve.

In an example, a torus or torus-section shaped neck bridge can have a central double-funnel, hourglass, and/or hyperbolic shaped opening through which hydrogels are inserted into the aneurysm sac. In an example, this device can further comprise a valve in the central opening of a torus or torus-section shaped neck bridge. In an example, a torus or torus-section shaped neck bridge can be created geometrically by rotating an upward-opening arc (e.g. a section of a circle). In an example, an intrasaccular aneurysm occlusion device with a torus shaped neck bridge can further comprise a catheter which is removably attached to a central opening in torus. In an example, a valve through an opening in a neck bridge can be in the cross-sectional center of the neck bridge. In an example, a torus or torus-section shaped neck bridge can have a central double-funnel, hourglass, and/or hyperbolic shaped opening. In an example, the cross-sectional area of the central opening in the torus or torus-section shaped neck bridge can be between 5% to 15% of the maximum cross-sectional area of the torus or torus-section shaped neck bridge.

In an example, a longitudinal flexible embolic member can be a helical coil. In an example, embolic members which are inserted through a neck bridge into an aneurysm sac can be string-of-pearls embolic members (e.g. longitudinal series of embolic components which are connected by a flexible filament or wire). In an example, a method for occluding a cerebral aneurysm can comprise: inserting a mesh or stent into an cerebral aneurysm, wherein the mesh or stent self-expands into a toroidal shape within the aneurysm sac; inserting embolic material or members into the distal portion of the aneurysm sac through an opening in the mesh or stent; and then closing the opening in the mesh or stent. In an example, a plurality of embolic members can be inserted into the aneurysm sac through a central opening and the neck bridge can have a sufficiently tight fit with the aneurysm walls that none of the embolic members escape into the parent blood vessel. In an example, embolic members (e.g. coils, hydrogels, microsponges, beads, or string-of-pearls strands) can be inserted into the aneurysm sac through a central opening in the neck bridge.

In an example, an intrasaccular aneurysm occlusion device with a torus shaped neck bridge can further comprise a catheter which passes through a central opening in torus to deliver embolic members and/or material into the aneurysm sac. In an example, an intrasaccular aneurysm occlusion device with a torus or torus-section shaped neck bridge can have two catheters: a first catheter to deliver the neck bride and a second catheter to deliver embolic members or material through an opening in the neck bridge.

In an example, an intrasaccular aneurysm occlusion device can comprise: a torus or torus-section shaped neck bridge which is configured to be inserted into an aneurysm sac and expand to cover the neck of the aneurysm; a catheter through which the neck bridge is delivered to the aneurysm sac; and a decoupling mechanism which connects the neck bridge to the catheter, wherein the decoupling mechanism can be remotely activated (e.g. to decouple the neck bridge and the catheter) by the device operator. In an example, an intrasaccular aneurysm occlusion device with a torus or torus-section shaped neck bridge can further comprise an electrical detachment mechanism.

In an example, a distal globular mesh can be separate from the neck bridge. In an example, a distal globular mesh can be attached to the outer perimeter of a torus or torus-section shaped neck bridge. In an example, a distal globular mesh can be attached to the neck bridge. In an example, a distal globular mesh can be attached to the distal surface of the neck bridge. In an example, the distal globular mesh can be made from a polymer and the torus or torus-section shaped neck bridge can be made from metal. In an example, a distal globular mesh can be made from a polymer and the neck bridge can be made from metal.

In an example, an intrasaccular aneurysm occlusion device can comprise: a torus-shaped neck bridge which is configured to be inserted into an aneurysm sac and then expanded to cover the neck of the aneurysm, wherein the shape of the neck bridge can be modeled by revolving a convex shape in three-dimensional space around an axis of revolution which is coplanar with the convex shape, wherein a convex shape diameter is the widest diameter of the convex shape which is perpendicular to the axis of revolution, wherein a revolution diameter is the widest diameter of the circle formed by the path of the center of the convex shape as the convex shape is revolved in three-dimensional space, and wherein the revolution diameter is greater than the convex shape diameter. In an example, the device can further comprise one or more embolic members or embolic material which is inserted into the aneurysm sac through a central opening in the neck bridge. In an example, the revolution diameter can be 5% to 30% greater than the convex shape diameter.

In an example, the convex shape can be a circle. In an example, the convex shape can be an ellipse with a longitudinal axis which is perpendicular to the axis of revolution. In an example, the convex shape can be an ellipse with a longitudinal axis which is parallel to the axis of revolution. In an example, the convex shape can be an ellipse wherein an extension of a longitudinal axis of the ellipse intersects the axis of revolution at an acute angle. In an example, the convex shape is the perimeter of the yin portion or the yang portion of a yin-yang symbol. In an example, the convex shape can be the perimeter of a teardrop shape.

In an example, an intrasaccular aneurysm occlusion device can comprise: a torus-section-shaped neck bridge which is configured to be inserted into an aneurysm sac and then expanded to cover the neck of the aneurysm, wherein the shape of the neck bridge can be modeled by revolving a section of a convex shape in three-dimensional space around an axis of revolution which is coplanar with the convex shape, wherein a convex shape diameter is the widest diameter of the convex shape which is perpendicular to the axis of revolution, wherein a revolution diameter is the widest diameter of the circle formed by the path of the center of the convex shape as the convex shape is revolved in three-dimensional space, and wherein the revolution diameter is greater than the convex shape diameter. In an example, the device can further comprise one or more embolic members or embolic material which is inserted into the aneurysm sac through a central opening in the neck bridge. In an example, the revolution diameter can be 5% to 30% greater than the convex shape diameter.

In an example, the section of a convex shape can be a semi-circle. In an example, the section of a convex shape can be the perimeter of a circle with a gap within a portion between 9 o'clock and 3 o'clock coordinates. In an example, the section of a convex shape can be the perimeter of a circle with a gap within a portion between 1 o'clock and 5 o'clock coordinates. In an example, the section of a convex shape can be a section of an ellipse with a longitudinal axis which is perpendicular to the axis of revolution. In an example, the section of a convex shape can be a section of an ellipse with a longitudinal axis which is parallel to the axis of revolution. In an example, the section of a convex shape can be a section of an ellipse wherein an extension of a longitudinal axis of the ellipse intersects the axis of revolution at an acute angle. In an example, the section of the convex shape can be the perimeter of an ellipse with a gap within a portion between 1 o'clock and 5 o'clock coordinates.

In an example, an intrasaccular aneurysm occlusion device can comprise: a longitudinal catheter that is configured to be inserted into a blood vessel, wherein this blood vessel is the parent vessel from which an aneurysm has formed; a longitudinal flexible embolic member that is configured to travel through the longitudinal lumen and be inserted into the aneurysm sac; and an expandable toroidal member that is configured to travel through the longitudinal lumen, be inserted into the aneurysm sac, and be expanded within the aneurysm sac; wherein this toroidal member is configured to substantially occlude the aneurysm neck after it is expanded; wherein the longitudinal flexible embolic member is inserted into the aneurysm sac through a central opening in the expandable toroidal member; and wherein the expandable toroidal member prevents the longitudinal flexible member from protruding into the parent vessel of the aneurysm.

Having completed the introductory section, this disclosure now describes the examples of intrasaccular aneurysm occlusion devices which are shown in FIGS. 1 through 37.

FIGS. 1 and 2 show an example of an intrasaccular aneurysm occlusion device comprising a torus or torus-section shaped neck bridge which is configured to be inserted into an aneurysm sac and then expanded to cover the neck of the aneurysm. FIG. 1 shows a wire-mesh, oblique-side-view diagram of the neck bridge. FIG. 2 illustrates how the shape of the neck bridge can be modeled by revolving a convex shape in three-dimensional space. This three-dimensional modeling shows how the shape of the device can be specified, but the device need not actually be made by revolving a convex shape in three-dimensions. The device can be made by 3D printing, weaving, braiding, tube inversion, or other methods.

Specifically, FIG. 1 shows an example of an intrasaccular aneurysm occlusion device comprising: a torus or torus-section shaped neck bridge 101 which is configured to be inserted into an aneurysm sac and then expanded to cover the neck of the aneurysm, wherein the shape of the neck bridge can be modeled by revolving a convex shape 201 in three-dimensional space around an axis of revolution 203 which is coplanar with the convex shape, wherein a convex shape diameter 202 is the widest diameter of the convex shape which is perpendicular to the axis of revolution, wherein a revolution diameter 204 is the widest diameter of the shape (e.g. circle) formed by the path of the center of the convex shape as the convex shape is revolved in three-dimensional space. In an example, this device can further comprise: a catheter to deliver the neck bridge to the aneurysm sac; embolic members or material which is inserted into the aneurysm sac through a central opening in the neck bridge; and/or a valve in the central opening which prevents embolic members or material from escaping.

In this example, the neck bridge is a complete torus. In this example, the convex shape 201 is a circle. In this example, the revolution diameter 204 is greater than the convex shape diameter 202. Relevant variations discussed elsewhere in this disclosure or in priority-linked disclosures can also be applied to this example.

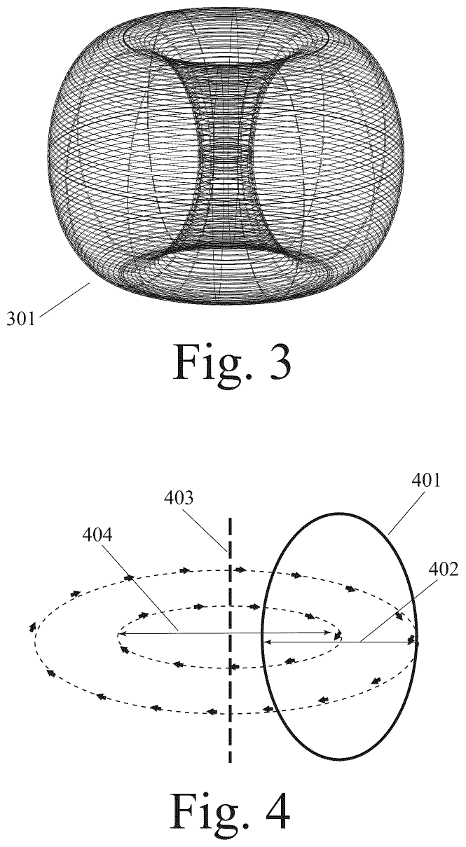

FIGS. 3 and 4 show an example of an intrasaccular aneurysm occlusion device comprising a torus or torus-section shaped neck bridge which is configured to be inserted into an aneurysm sac and then expanded to cover the neck of the aneurysm. FIG. 3 shows a wire-mesh, oblique-side-view diagram of the neck bridge. FIG. 4 illustrates how the shape of the neck bridge can be modeled by revolving a convex shape in three-dimensional space. This three-dimensional modeling shows how the shape of the device can be specified, but the device need not actually be made by revolving a convex shape in three-dimensions. The device can be made by 3D printing, weaving, braiding, tube inversion, or other methods.

Specifically, FIG. 3 shows an example of an intrasaccular aneurysm occlusion device comprising: a torus or torus-section shaped neck bridge 301 which is configured to be inserted into an aneurysm sac and then expanded to cover the neck of the aneurysm, wherein the shape of the neck bridge can be modeled by revolving a convex shape 401 in three-dimensional space around an axis of revolution 403 which is coplanar with the convex shape, wherein a convex shape diameter 402 is the widest diameter of the convex shape which is perpendicular to the axis of revolution, wherein a revolution diameter 404 is the widest diameter of the shape (e.g. circle) formed by the path of the center of the convex shape as the convex shape is revolved in three-dimensional space. In an example, this device can further comprise: a catheter to deliver the neck bridge to the aneurysm sac; embolic members or material which is inserted into the aneurysm sac through a central opening in the neck bridge; and/or a valve in the central opening which prevents embolic members or material from escaping.

In this example, the neck bridge is a complete torus. In this example, the convex shape 401 is an ellipse whose longest axis is parallel to the axis of revolution 403. In this example, the revolution diameter 404 is greater than the convex shape diameter 402. Relevant variations discussed elsewhere in this disclosure or in priority-linked disclosures can also be applied to this example.

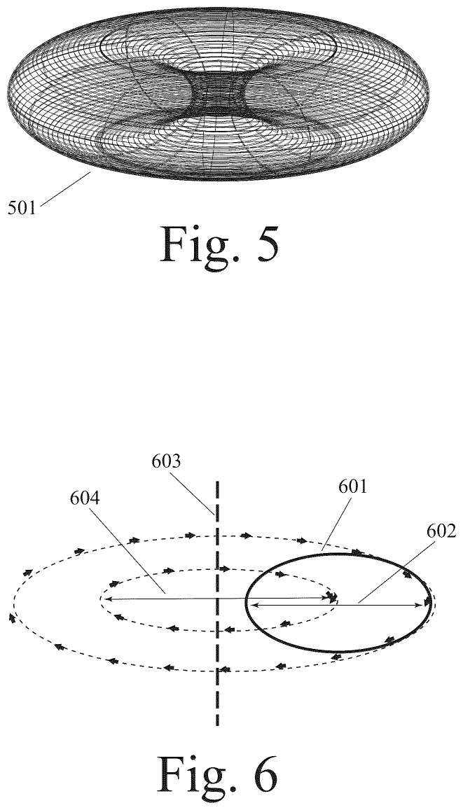

FIGS. 5 and 6 show an example of an intrasaccular aneurysm occlusion device comprising a torus or torus-section shaped neck bridge which is configured to be inserted into an aneurysm sac and then expanded to cover the neck of the aneurysm. FIG. 5 shows a wire-mesh, oblique-side-view diagram of the neck bridge. FIG. 6 illustrates how the shape of the neck bridge can be modeled by revolving a convex shape in three-dimensional space. This three-dimensional modeling shows how the shape of the device can be specified, but the device need not actually be made by revolving a convex shape in three-dimensions. The device can be made by 3D printing, weaving, braiding, tube inversion, or other methods.

Specifically, FIG. 5 shows an example of an intrasaccular aneurysm occlusion device comprising: a torus or torus-section shaped neck bridge 501 which is configured to be inserted into an aneurysm sac and then expanded to cover the neck of the aneurysm, wherein the shape of the neck bridge can be modeled by revolving a convex shape 601 in three-dimensional space around an axis of revolution 603 which is coplanar with the convex shape, wherein a convex shape diameter 602 is the widest diameter of the convex shape which is perpendicular to the axis of revolution, wherein a revolution diameter 604 is the widest diameter of the shape (e.g. circle) formed by the path of the center of the convex shape as the convex shape is revolved in three-dimensional space. In an example, this device can further comprise: a catheter to deliver the neck bridge to the aneurysm sac; embolic members or material which is inserted into the aneurysm sac through a central opening in the neck bridge; and/or a valve in the central opening which prevents embolic members or material from escaping.

In this example, the neck bridge is a complete torus. In this example, the convex shape 601 is an ellipse whose longest axis is perpendicular to the axis of revolution 603. In this example, the revolution diameter 604 is greater than the convex shape diameter 602. Relevant variations discussed elsewhere in this disclosure or in priority-linked disclosures can also be applied to this example.

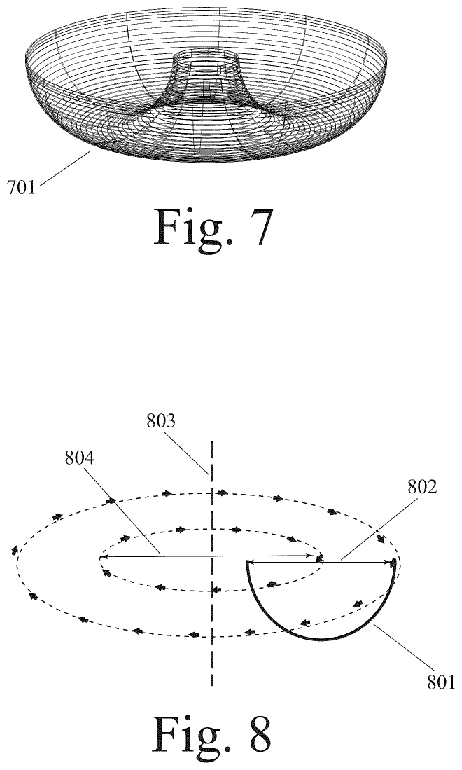

FIGS. 7 and 8 show an example of an intrasaccular aneurysm occlusion device comprising a torus or torus-section shaped neck bridge which is configured to be inserted into an aneurysm sac and then expanded to cover the neck of the aneurysm. FIG. 7 shows a wire-mesh, oblique-side-view diagram of the neck bridge. FIG. 8 illustrates how the shape of the neck bridge can be modeled by revolving a section of a convex shape in three-dimensional space. This three-dimensional modeling shows how the shape of the device can be specified, but the device need not actually be made by revolving a convex shape in three-dimensions. The device can be made by 3D printing, weaving, braiding, tube inversion, or other methods.

Specifically, FIG. 7 shows an example of an intrasaccular aneurysm occlusion device comprising: a torus or torus-section shaped neck bridge 701 which is configured to be inserted into an aneurysm sac and then expanded to cover the neck of the aneurysm, wherein the shape of the neck bridge can be modeled by revolving a section of a convex shape 801 in three-dimensional space around an axis of revolution 803 which is coplanar with the convex shape, wherein a convex shape diameter 802 is the widest diameter of the convex shape which is perpendicular to the axis of revolution, wherein a revolution diameter 804 is the widest diameter of the shape (e.g. circle) formed by the path of the center of the convex shape as the convex shape is revolved in three-dimensional space. In an example, this device can further comprise: a catheter to deliver the neck bridge to the aneurysm sac; embolic members or material which is inserted into the aneurysm sac through a central opening in the neck bridge; and/or a valve in the central opening which prevents embolic members or material from escaping.

In this example, the neck bridge is a section of a torus. In this example, the neck bridge is the lower half of a torus. In this example, the convex shape 801 is a circle. In this example, the section of the convex shape is the lower half of the circle, which is a semicircle between the 3 o'clock and 9 o'clock coordinates on the circle. In this example, the revolution diameter 804 is greater than the convex shape diameter 802. Relevant variations discussed elsewhere in this disclosure or in priority-linked disclosures can also be applied to this example.

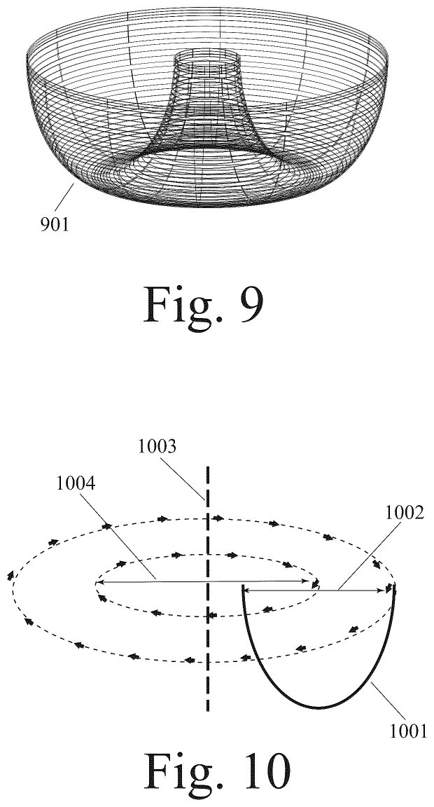

FIGS. 9 and 10 show an example of an intrasaccular aneurysm occlusion device comprising a torus or torus-section shaped neck bridge which is configured to be inserted into an aneurysm sac and then expanded to cover the neck of the aneurysm. FIG. 9 shows a wire-mesh, oblique-side-view diagram of the neck bridge. FIG. 10 illustrates how the shape of the neck bridge can be modeled by revolving a section of a convex shape in three-dimensional space. This three-dimensional modeling shows how the shape of the device can be specified, but the device need not actually be made by revolving a convex shape in three-dimensions. The device can be made by 3D printing, weaving, braiding, tube inversion, or other methods.

Specifically, FIG. 9 shows an example of an intrasaccular aneurysm occlusion device comprising: a torus or torus-section shaped neck bridge 901 which is configured to be inserted into an aneurysm sac and then expanded to cover the neck of the aneurysm, wherein the shape of the neck bridge can be modeled by revolving a section of a convex shape 1001 in three-dimensional space around an axis of revolution 1003 which is coplanar with the convex shape, wherein a convex shape diameter 1002 is the widest diameter of the convex shape which is perpendicular to the axis of revolution, wherein a revolution diameter 1004 is the widest diameter of the shape (e.g. circle) formed by the path of the center of the convex shape as the convex shape is revolved in three-dimensional space. In an example, this device can further comprise: a catheter to deliver the neck bridge to the aneurysm sac; embolic members or material which is inserted into the aneurysm sac through a central opening in the neck bridge; and/or a valve in the central opening which prevents embolic members or material from escaping.

In this example, the neck bridge is a section of a torus. In this example, the neck bridge is the lower half of a torus. This shape is similar to the shape of a mold for a Bundt cake. In this example, the convex shape 1001 is an ellipse whose longest dimension is an ellipse whose longest axis is parallel to the axis of revolution 1003. In this example, the section of the convex shape is the lower half of the ellipse, between the 3 o'clock and 9 o'clock coordinates on the ellipse. In this example, the revolution diameter 1004 is greater than the convex shape diameter 1002. Relevant variations discussed elsewhere in this disclosure or in priority-linked disclosures can also be applied to this example.

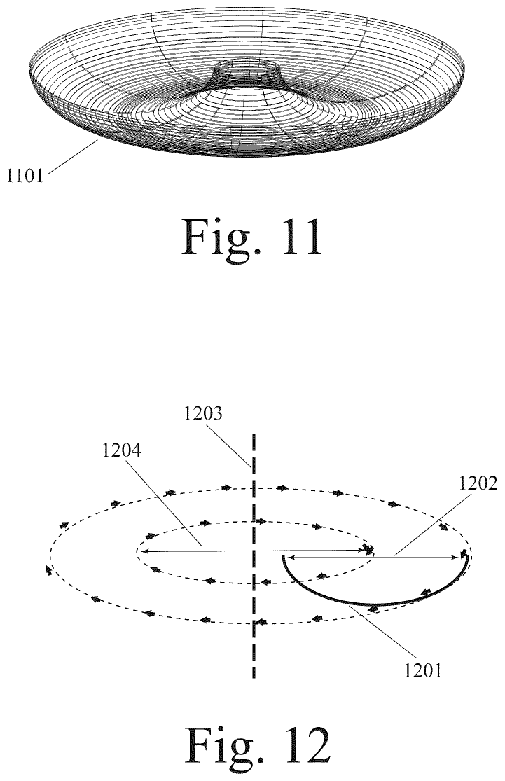

FIGS. 11 and 12 show an example of an intrasaccular aneurysm occlusion device comprising a torus or torus-section shaped neck bridge which is configured to be inserted into an aneurysm sac and then expanded to cover the neck of the aneurysm. FIG. 11 shows a wire-mesh, oblique-side-view diagram of the neck bridge. FIG. 12 illustrates how the shape of the neck bridge can be modeled by revolving a section of a convex shape in three-dimensional space. This three-dimensional modeling shows how the shape of the device can be specified, but the device need not actually be made by revolving a convex shape in three-dimensions. The device can be made by 3D printing, weaving, braiding, tube inversion, or other methods.

Specifically, FIG. 11 shows an example of an intrasaccular aneurysm occlusion device comprising: a torus or torus-section shaped neck bridge 1101 which is configured to be inserted into an aneurysm sac and then expanded to cover the neck of the aneurysm, wherein the shape of the neck bridge can be modeled by revolving a section of a convex shape 1201 in three-dimensional space around an axis of revolution 1203 which is coplanar with the convex shape, wherein a convex shape diameter 1202 is the widest diameter of the convex shape which is perpendicular to the axis of revolution, wherein a revolution diameter 1204 is the widest diameter of the shape (e.g. circle) formed by the path of the center of the convex shape as the convex shape is revolved in three-dimensional space. In an example, this device can further comprise: a catheter to deliver the neck bridge to the aneurysm sac; embolic members or material which is inserted into the aneurysm sac through a central opening in the neck bridge; and/or a valve in the central opening which prevents embolic members or material from escaping.

In this example, the neck bridge is a section of a torus. In this example, the neck bridge is the lower half of a torus. In this example, the convex shape 1201 is an ellipse whose longest dimension is an ellipse whose longest axis is perpendicular to the axis of revolution 1203. In this example, the section of the convex shape is the lower half of the ellipse, spanning between the 3 o'clock and 9 o'clock coordinates on the ellipse. In this example, the revolution diameter 1204 is greater than the convex shape diameter 1202. Relevant variations discussed elsewhere in this disclosure or in priority-linked disclosures can also be applied to this example.

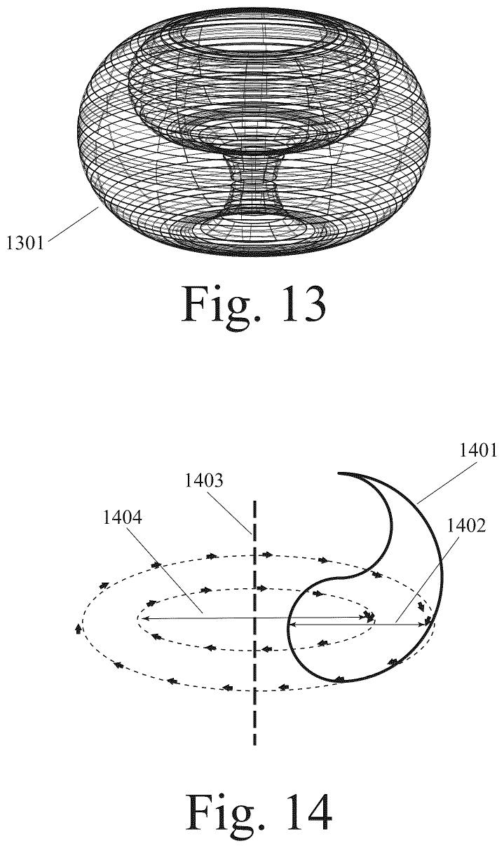

FIGS. 13 and 14 show an example of an intrasaccular aneurysm occlusion device comprising a torus or torus-section shaped neck bridge which is configured to be inserted into an aneurysm sac and then expanded to cover the neck of the aneurysm. FIG. 13 shows a wire-mesh, oblique-side-view diagram of the neck bridge. FIG. 14 illustrates how the shape of the neck bridge can be modeled by revolving a convex shape in three-dimensional space. This three-dimensional modeling shows how the shape of the device can be specified, but the device need not actually be made by revolving a convex shape in three-dimensions. The device can be made by 3D printing, weaving, braiding, tube inversion, or other methods.

Specifically, FIG. 13 shows an example of an intrasaccular aneurysm occlusion device comprising: a torus or torus-section shaped neck bridge 1301 which is configured to be inserted into an aneurysm sac and then expanded to cover the neck of the aneurysm, wherein the shape of the neck bridge can be modeled by revolving a convex shape 1401 in three-dimensional space around an axis of revolution 1403 which is coplanar with the convex shape, wherein a convex shape diameter 1402 is the widest diameter of the convex shape which is perpendicular to the axis of revolution, wherein a revolution diameter 1404 is the widest diameter of the shape (e.g. circle) formed by the path of the center of the convex shape as the convex shape is revolved in three-dimensional space. In an example, this device can further comprise: a catheter to deliver the neck bridge to the aneurysm sac; embolic members or material which is inserted into the aneurysm sac through a central opening in the neck bridge; and/or a valve in the central opening which prevents embolic members or material from escaping.

In this example, the neck bridge is a complete torus. In this example, the convex shape 1401 is one half (e.g. yin or yang) of a circular yin-yang symbol. In this example, the revolution diameter 1404 is greater than the convex shape diameter 1402. Relevant variations discussed elsewhere in this disclosure or in priority-linked disclosures can also be applied to this example.

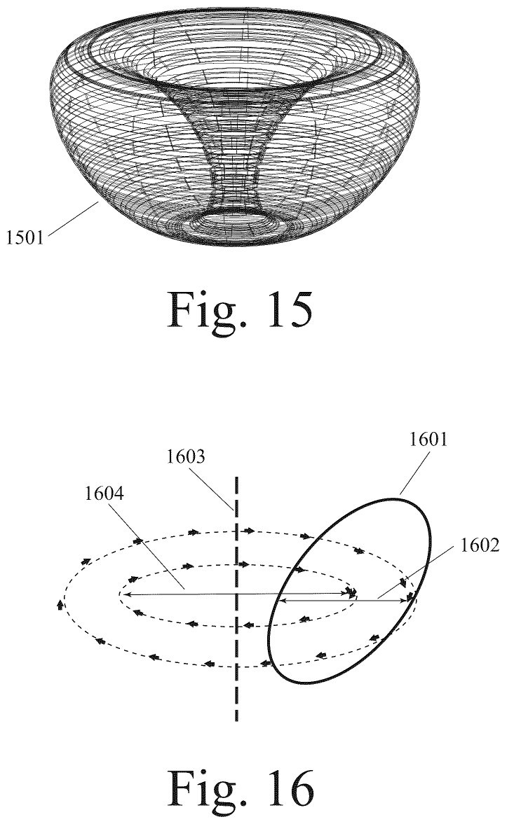

FIGS. 15 and 16 show an example of an intrasaccular aneurysm occlusion device comprising a torus or torus-section shaped neck bridge which is configured to be inserted into an aneurysm sac and then expanded to cover the neck of the aneurysm. FIG. 15 shows a wire-mesh, oblique-side-view diagram of the neck bridge. FIG. 16 illustrates how the shape of the neck bridge can be modeled by revolving a convex shape in three-dimensional space. This three-dimensional modeling shows how the shape of the device can be specified, but the device need not actually be made by revolving a convex shape in three-dimensions. The device can be made by 3D printing, weaving, braiding, tube inversion, or other methods.

Specifically, FIG. 15 shows an example of an intrasaccular aneurysm occlusion device comprising: a torus or torus-section shaped neck bridge 1501 which is configured to be inserted into an aneurysm sac and then expanded to cover the neck of the aneurysm, wherein the shape of the neck bridge can be modeled by revolving a convex shape 1601 in three-dimensional space around an axis of revolution 1603 which is coplanar with the convex shape, wherein a convex shape diameter 1602 is the widest diameter of the convex shape which is perpendicular to the axis of revolution, wherein a revolution diameter 1604 is the widest diameter of the shape (e.g. circle) formed by the path of the center of the convex shape as the convex shape is revolved in three-dimensional space. In an example, this device can further comprise: a catheter to deliver the neck bridge to the aneurysm sac; embolic members or material which is inserted into the aneurysm sac through a central opening in the neck bridge; and/or a valve in the central opening which prevents embolic members or material from escaping.

In this example, the neck bridge is a complete torus. In this example, the convex shape 1601 is an ellipse. In this example, an extension of the longitudinal axis of the ellipse intersects the axis of revolution at an acute angle. In this example, the distal end of the ellipse is farther from the axis of revolution than the proximal end of the ellipse. In this example, the revolution diameter 1604 is greater than the convex shape diameter 1602. Relevant variations discussed elsewhere in this disclosure or in priority-linked disclosures can also be applied to this example.

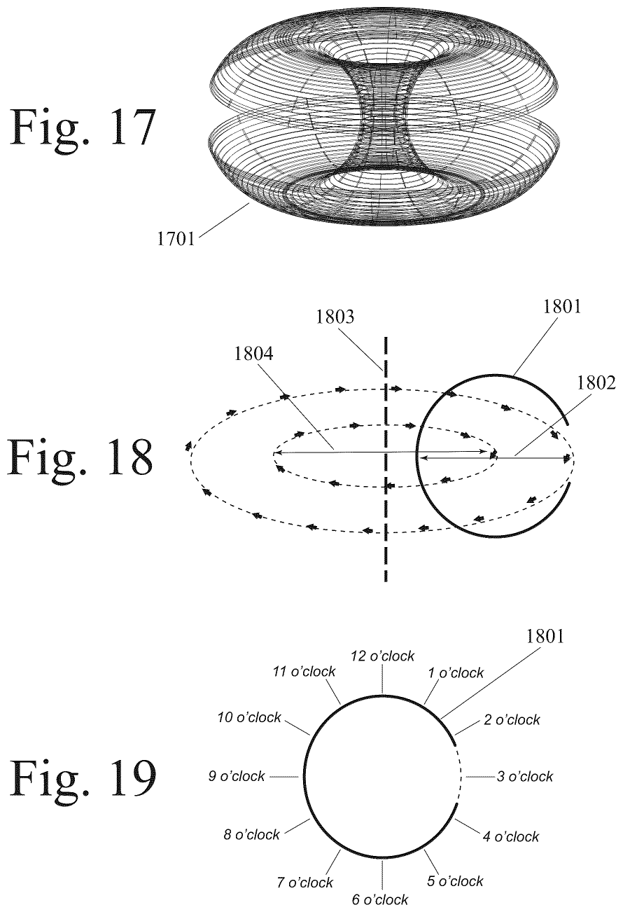

FIGS. 17 through 19 show an example of an intrasaccular aneurysm occlusion device comprising a torus or torus-section shaped neck bridge which is configured to be inserted into an aneurysm sac and then expanded to cover the neck of the aneurysm. FIG. 17 shows a wire-mesh, oblique-side-view diagram of the neck bridge. FIG. 18 illustrates how the shape of the neck bridge can be modeled by revolving a section of a convex shape in three-dimensional space. This three-dimensional modeling shows how the shape of the device can be specified, but the device need not actually be made by revolving a convex shape in three-dimensions. The device can be made by 3D printing, weaving, braiding, tube inversion, or other methods. FIG. 19 illustrates the locations of clock-hour coordinates around the perimeter of the convex shape. These coordinates are useful for specifying the location of a gap in the convex shape, wherein this gap creates the section of the convex shape which is revolved.

Specifically, FIG. 17 shows an example of an intrasaccular aneurysm occlusion device comprising: a torus or torus-section shaped neck bridge 1701 which is configured to be inserted into an aneurysm sac and then expanded to cover the neck of the aneurysm, wherein the shape of the neck bridge can be modeled by revolving a section of a convex shape 1801 in three-dimensional space around an axis of revolution 1803 which is coplanar with the convex shape, wherein a convex shape diameter 1802 is the widest diameter of the convex shape which is perpendicular to the axis of revolution, wherein a revolution diameter 1804 is the widest diameter of the shape (e.g. circle) formed by the path of the center of the convex shape as the convex shape is revolved in three-dimensional space. In an example, this device can further comprise: a catheter to deliver the neck bridge to the aneurysm sac; embolic members or material which is inserted into the aneurysm sac through a central opening in the neck bridge; and/or a valve in the central opening which prevents embolic members or material from escaping.

In this example, the neck bridge is a section of a torus. In this example, the neck bridge is shaped like a torus with a central circumferential gap between its proximal and distal surfaces. This shape of this torus section is similar to the shape of a yo-yo toy. This shape can be particularly useful for occluding an aneurysm sac with an arcuate (e.g. bent) central axis. In this example, the section of the convex shape which is revolved is shaped like the letter “C”. As shown in FIG. 19, this section is a circle with a gap between the 2 o'clock and 4 o'clock coordinates (but not spanning the full distance between these coordinates). In this example, the revolution diameter is greater than the convex shape diameter. Relevant variations discussed elsewhere in this disclosure or in priority-linked disclosures can also be applied to this example.

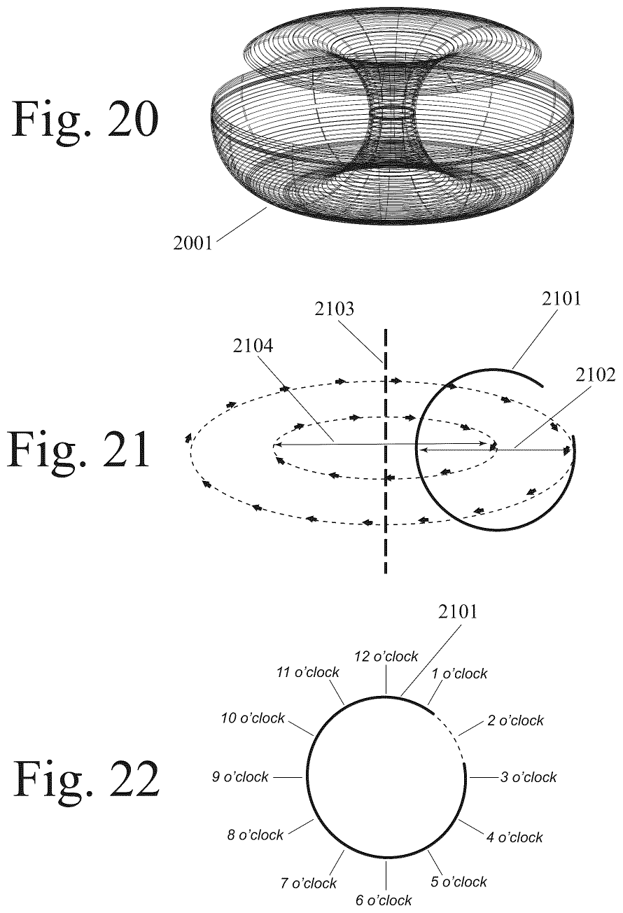

FIGS. 20 through 22 show an example of an intrasaccular aneurysm occlusion device comprising a torus or torus-section shaped neck bridge which is configured to be inserted into an aneurysm sac and then expanded to cover the neck of the aneurysm. FIG. 20 shows a wire-mesh, oblique-side-view diagram of the neck bridge. FIG. 21 illustrates how the shape of the neck bridge can be modeled by revolving a section of a convex shape in three-dimensional space. This three-dimensional modeling shows how the shape of the device can be specified, but the device need not actually be made by revolving a convex shape in three-dimensions. The device can be made by 3D printing, weaving, braiding, tube inversion, or other methods. FIG. 22 illustrates the locations of clock-hour coordinates around the perimeter of the convex shape. These coordinates are useful for specifying the location of a gap in the convex shape, wherein this gap creates the section of the convex shape which is revolved.

Specifically, FIG. 20 shows an example of an intrasaccular aneurysm occlusion device comprising: a torus or torus-section shaped neck bridge 2001 which is configured to be inserted into an aneurysm sac and then expanded to cover the neck of the aneurysm, wherein the shape of the neck bridge can be modeled by revolving a section of a convex shape 2101 in three-dimensional space around an axis of revolution 2103 which is coplanar with the convex shape, wherein a convex shape diameter 2102 is the widest diameter of the convex shape which is perpendicular to the axis of revolution, wherein a revolution diameter 2104 is the widest diameter of the shape (e.g. circle) formed by the path of the center of the convex shape as the convex shape is revolved in three-dimensional space. In an example, this device can further comprise: a catheter to deliver the neck bridge to the aneurysm sac; embolic members or material which is inserted into the aneurysm sac through a central opening in the neck bridge; and/or a valve in the central opening which prevents embolic members or material from escaping.

In this example, the neck bridge is a section of a torus. In this example, the neck bridge is shaped like a torus with an upper-circumferential gap between its proximal and distal surfaces. The shape of this torus section shape is somewhat similar to those of Ancient Roman and/or Italian plaza water fountains. This shape can be particularly useful for occluding an aneurysm sac with an arcuate (e.g. bent) central axis. In this example, the section of the convex shape which is revolved has a shape like the letter “C” which has been rotated counter-clockwise by approximately 30 degrees. As shown in FIG. 22, this section is a circle with a gap between the 1 o'clock and 3 o'clock coordinates (but not spanning the full distance between these coordinates). In this example, the revolution diameter is greater than the convex shape diameter. Relevant variations discussed elsewhere in this disclosure or in priority-linked disclosures can also be applied to this example.

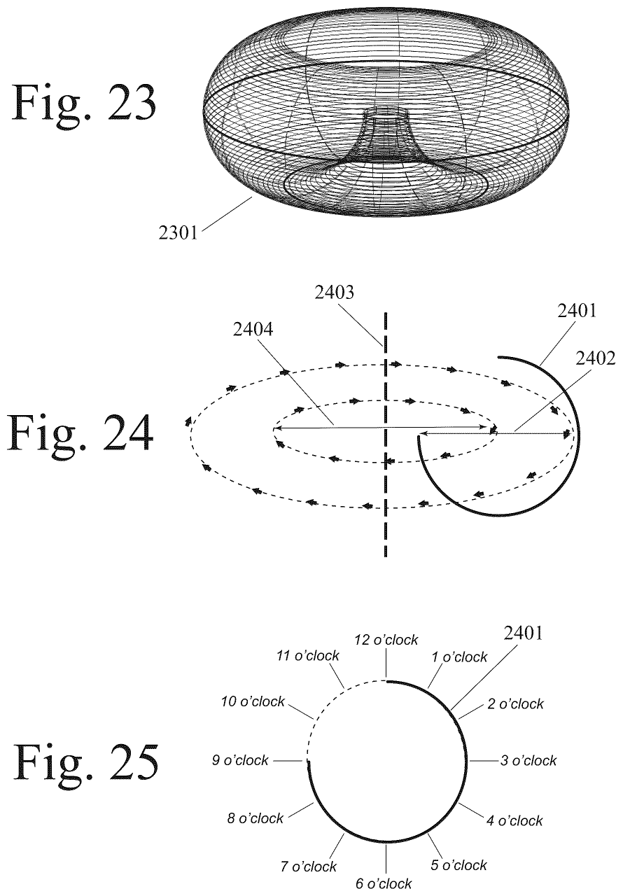

FIGS. 23 through 25 show an example of an intrasaccular aneurysm occlusion device comprising a torus or torus-section shaped neck bridge which is configured to be inserted into an aneurysm sac and then expanded to cover the neck of the aneurysm. FIG. 23 shows a wire-mesh, oblique-side-view diagram of the neck bridge. FIG. 24 illustrates how the shape of the neck bridge can be modeled by revolving a section of a convex shape in three-dimensional space. This three-dimensional modeling shows how the shape of the device can be specified, but the device need not actually be made by revolving a convex shape in three-dimensions. The device can be made by 3D printing, weaving, braiding, tube inversion, or other methods. FIG. 25 illustrates the locations of clock-hour coordinates around the perimeter of the convex shape. These coordinates are useful for specifying the location of a gap in the convex shape, wherein this gap creates the section of the convex shape which is revolved.

Specifically, FIG. 23 shows an example of an intrasaccular aneurysm occlusion device comprising: a torus or torus-section shaped neck bridge 2301 which is configured to be inserted into an aneurysm sac and then expanded to cover the neck of the aneurysm, wherein the shape of the neck bridge can be modeled by revolving a section of a convex shape 2401 in three-dimensional space around an axis of revolution 2403 which is coplanar with the convex shape, wherein a convex shape diameter 2402 is the widest diameter of the convex shape which is perpendicular to the axis of revolution, wherein a revolution diameter 2404 is the widest diameter of the shape (e.g. circle) formed by the path of the center of the convex shape as the convex shape is revolved in three-dimensional space. In an example, this device can further comprise: a catheter to deliver the neck bridge to the aneurysm sac; embolic members or material which is inserted into the aneurysm sac through a central opening in the neck bridge; and/or a valve in the central opening which prevents embolic members or material from escaping.

In this example, the neck bridge is a section of a torus. In this example, the neck bridge is shaped like a torus with a central circular opening in its distal surface. As shown in FIG. 25, the revolved section is a circle with a gap between the 9 o'clock and 12 o'clock coordinates. In this example, the revolution diameter is greater than the convex shape diameter. Relevant variations discussed elsewhere in this disclosure or in priority-linked disclosures can also be applied to this example.

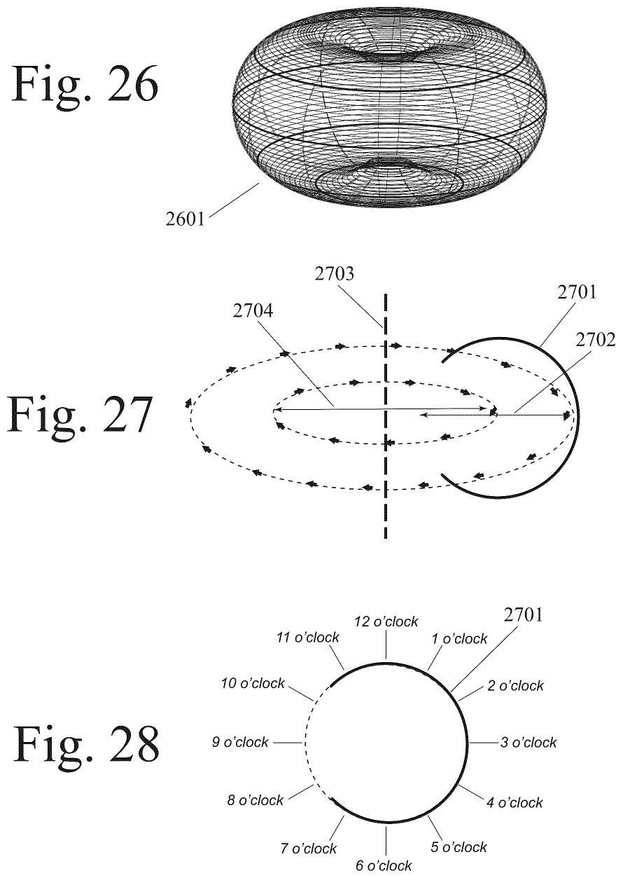

FIGS. 26 through 28 show an example of an intrasaccular aneurysm occlusion device comprising a torus or torus-section shaped neck bridge which is configured to be inserted into an aneurysm sac and then expanded to cover the neck of the aneurysm. FIG. 26 shows a wire-mesh, oblique-side-view diagram of the neck bridge. FIG. 27 illustrates how the shape of the neck bridge can be modeled by revolving a section of a convex shape in three-dimensional space. This three-dimensional modeling shows how the shape of the device can be specified, but the device need not actually be made by revolving a convex shape in three-dimensions. The device can be made by 3D printing, weaving, braiding, tube inversion, or other methods. FIG. 28 illustrates the locations of clock-hour coordinates around the perimeter of the convex shape. These coordinates are useful for specifying the location of a gap in the convex shape, wherein this gap creates the section of the convex shape which is revolved.

Specifically, FIG. 26 shows an example of an intrasaccular aneurysm occlusion device comprising: a torus or torus-section shaped neck bridge 2601 which is configured to be inserted into an aneurysm sac and then expanded to cover the neck of the aneurysm, wherein the shape of the neck bridge can be modeled by revolving a section of a convex shape 2701 in three-dimensional space around an axis of revolution 2703 which is coplanar with the convex shape, wherein a convex shape diameter 2702 is the widest diameter of the convex shape which is perpendicular to the axis of revolution, wherein a revolution diameter 2704 is the widest diameter of the shape (e.g. circle) formed by the path of the center of the convex shape as the convex shape is revolved in three-dimensional space. In an example, this device can further comprise: a catheter to deliver the neck bridge to the aneurysm sac; embolic members or material which is inserted into the aneurysm sac through a central opening in the neck bridge; and/or a valve in the central opening which prevents embolic members or material from escaping.

In this example, the neck bridge is a section of a torus. As shown in FIG. 28, the revolved section is a circle with a gap between the 7 o'clock and 11 o'clock coordinates. This shape is similar to the classic shape of a pumpkin. In this example, the revolution diameter is greater than the convex shape diameter. Relevant variations discussed elsewhere in this disclosure or in priority-linked disclosures can also be applied to this example.

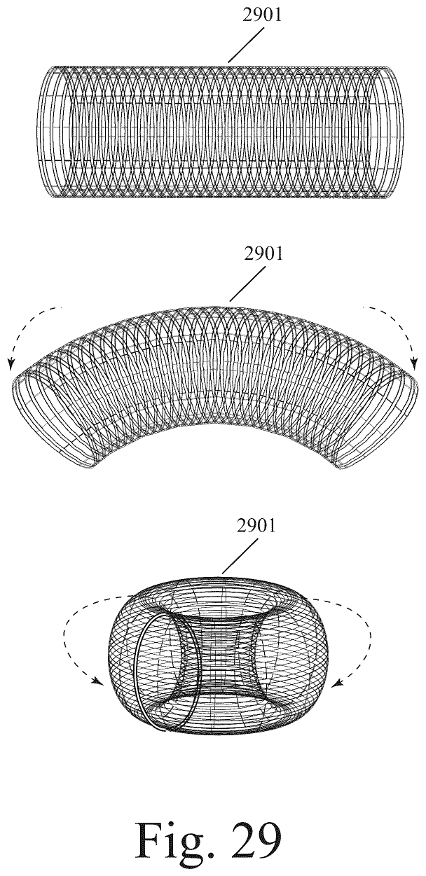

FIG. 29 shows an example of one method for making a torus or torus-section shaped neck bridge. In this example, the neck bridge is made by curving a tubular mesh 2901. The upper-third of FIG. 29 shows the tubular mesh before it is curved. The middle-third of FIG. 29 shows the longitudinal axis of the tubular mesh after it has been curved into an arc. The lower-third of FIG. 29 shows the longitudinal axis of the tubular mesh having been curved into a complete circle and the ends of the tubular mesh having been connected to each other, thereby forming a torus-shaped neck bridge. In an example, a method for making a torus-shaped neck bridge can comprise: curving a tubular mesh so that its longitudinal axis becomes circular; and then connecting the ends of the tubular mesh to each other.

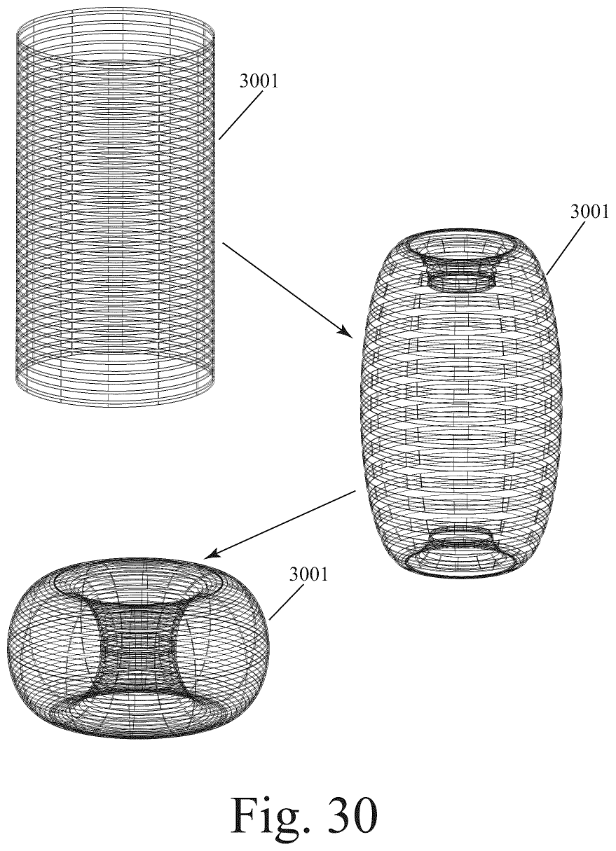

FIG. 30 shows an example of another method for making a torus or torus-section shaped neck bridge from a tubular mesh 3001. The upper-left third of FIG. 30 shows a tubular mesh before inversion. The middle-right of FIG. 30 shows the ends of the tubular mesh being inverted into the tube. The lower-left of FIG. 30 shows the ends of the tube having been further inverted and connected to each other, thereby forming a torus-shaped neck bridge. In an example, a method for making a torus-shaped neck bridge can comprise: inverting the ends of a tubular mesh into the tube; and then connecting the ends of the tubular mesh to each other within the tube. In other examples, a torus or torus-section shaped neck bridge can be made by another method such as 3D printing, weaving, braiding, or laser cutting.

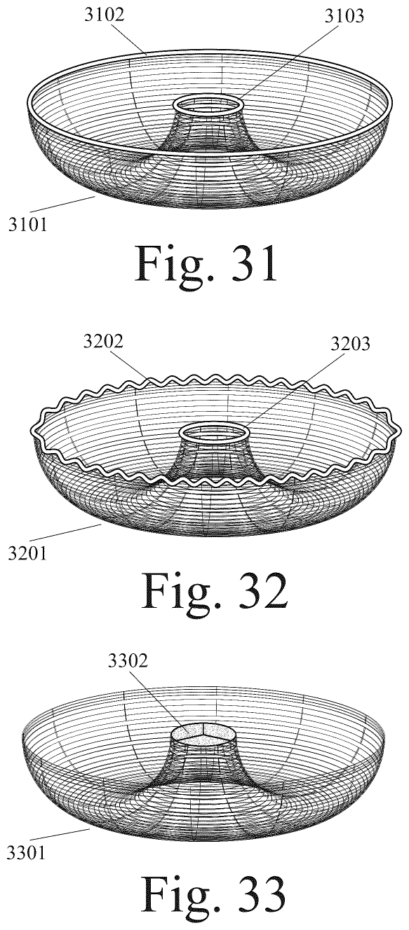

FIG. 31 shows an example of an intrasaccular aneurysm occlusion device comprising: a torus or torus-section shaped neck bridge 3101; wherein the neck bridge further comprises a first reinforcing ring 3102 (e.g. thicker wire or band) around its widest circumference; and wherein the neck bridge further comprises a second reinforcing ring 3103 (e.g. thicker wire or band) around the distal end of a central opening. This neck bridge is configured to be inserted into an aneurysm sac and then expanded to cover the neck of the aneurysm. Relevant variations discussed elsewhere in this disclosure or in priority-linked disclosures can also be applied to this example.

FIG. 32 shows an example of an intrasaccular aneurysm occlusion device comprising: a torus or torus-section shaped neck bridge 3201; wherein the neck bridge further comprises an undulating, sinusoidal, and/or zigzagging reinforcing ring 3202 (e.g. thicker wire or band) around its widest circumference; and wherein the neck bridge further comprises a second reinforcing ring 3203 (e.g. thicker wire or band) around the distal end of a central opening of the neck bridge. This neck bridge is configured to be inserted into an aneurysm sac and then expanded to cover the neck of the aneurysm. Relevant variations discussed elsewhere in this disclosure or in priority-linked disclosures can also be applied to this example.

FIG. 33 shows an example of an intrasaccular aneurysm occlusion device comprising: a torus or torus-section shaped neck bridge 3301; wherein the neck bridge further comprises a valve 3302 in (or on) a central opening of the neck bridge. This neck bridge is configured to be inserted into an aneurysm sac and then expanded to cover the neck of the aneurysm. In an example, this valve can be a passive valve which opens and/or closes without action by the operator of the device. In an example, this valve can be an active valve which is remotely opened and/or closed by the operator of the device (e.g. by applying electrical energy, pushing or pulling a wire, or rotation a wire). Relevant variations discussed elsewhere in this disclosure or in priority-linked disclosures can also be applied to this example.

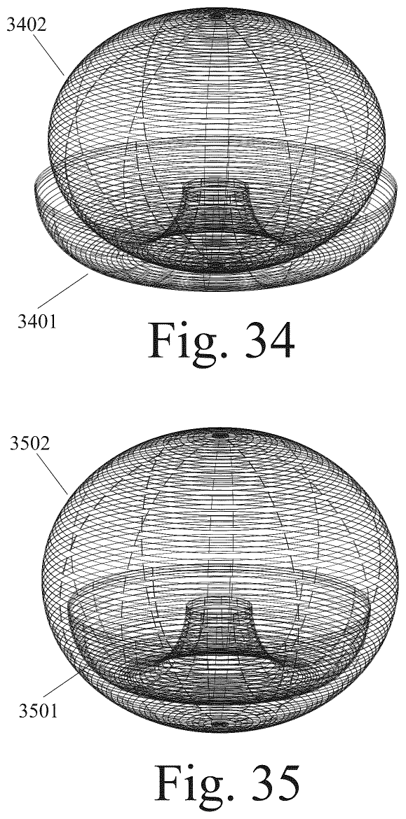

FIG. 34 shows a wire-mesh, oblique-side-view diagram of an intrasaccular aneurysm occlusion device comprising: a proximal torus or torus-section shaped neck bridge 3401 which is configured to be inserted into an aneurysm sac and then expanded to cover the neck of the aneurysm; and a distal globular mesh or net 3402, wherein a portion (e.g. less than 50%) of the distal globular mesh or net is nested within the proximal torus or torus-section shaped neck bridge. In an example, embolic members and/or material can be inserted through a central opening in the neck bridge into the distal global mesh or net. Relevant variations discussed elsewhere in this disclosure or in priority-linked disclosures can also be applied to this example.

FIG. 35 shows a wire-mesh, oblique-side-view diagram of an intrasaccular aneurysm occlusion device comprising: a proximal torus or torus-section shaped neck bridge 3501 which is configured to be inserted into an aneurysm sac and then expanded to cover the neck of the aneurysm; and a distal globular mesh or net 3502, wherein the proximal torus or torus-section shaped neck bridge is inside the distal globular mesh or net. In an example, embolic members and/or material can be inserted through a central opening in the neck bridge into the distal global mesh or net. Relevant variations discussed elsewhere in this disclosure or in priority-linked disclosures can also be applied to this example.

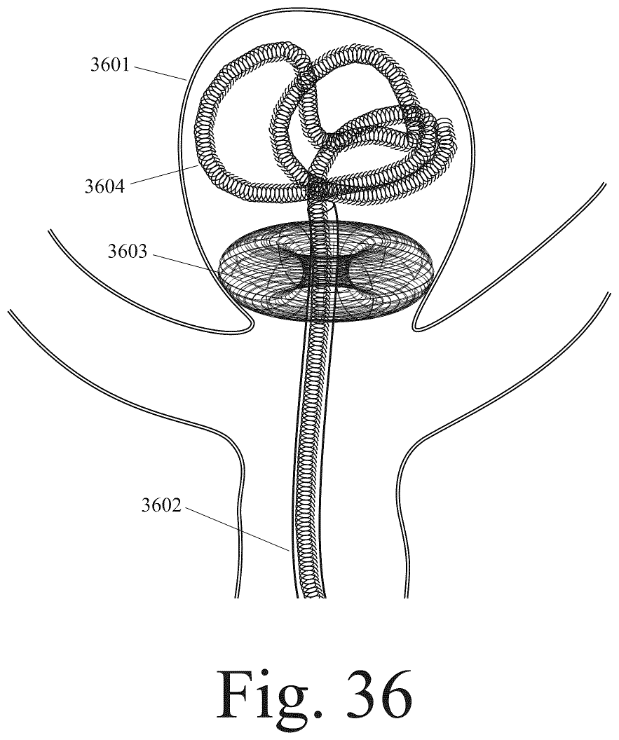

FIG. 36 shows an example of a device to occlude an aneurysm comprising: a longitudinal catheter 3602 that is configured to be inserted into a blood vessel, wherein this blood vessel is the parent vessel from which an aneurysm 3601 has formed; a longitudinal flexible embolic member 3604 (e.g. embolic coil) that is configured to travel through the longitudinal lumen and be inserted into the aneurysm sac; and an expandable toroidal member 3603 (e.g. neck bridge) that is configured to travel through the longitudinal lumen, be inserted into the aneurysm sac, and be expanded within the aneurysm sac; wherein this toroidal member is configured to substantially occlude the aneurysm neck after it is expanded; wherein the longitudinal flexible embolic member is inserted into the aneurysm sac through a central opening in the expandable toroidal member; and wherein the expandable toroidal member prevents the longitudinal flexible member from protruding into the parent vessel of the aneurysm.

In this example, the longitudinal flexible embolic member is a coil. In this example, the toroidal member (e.g. neck bridge) self-expands. In an example, the toroidal member can comprise shape memory material. In an example, a plurality of embolic members can be inserted into the aneurysm sac through a central opening of the toroidal member (e.g. neck bridge). In an example the toroidal member can have a sufficiently tight fit with the aneurysm walls that none of the embolic members escape into the parent blood vessel. In an example, this device can further comprise a valve in the central opening of the toroidal member.

In an example, a proximal torus (e.g. torus-shaped neck bridge) can comprise a mesh, network, lattice, or radial array of wires or other stiff fibers. In an example, embolic members can be inserted through (a one-way valve in) a central opening (e.g. hole) in a proximal torus. In an example, the central opening of a proximal torus can be closed after embolic members have been inserted through it. In an example, a method for occluding a cerebral aneurysm can comprise: inserting a mesh or stent into an cerebral aneurysm, wherein the mesh or stent self-expands into a toroidal shape within the aneurysm sac; inserting embolic material or members into the distal portion of the aneurysm sac through an opening in the mesh or stent; and then closing the opening in the mesh or stent.

In an example, an intrasaccular aneurysm occlusion device can comprise: a catheter; one or more embolic coils which are inserted through the catheter into an aneurysm sac; a toroidal neck bridge (e.g. stent, lattice, mesh, or framework) which expands (e.g. self-expands) within the aneurysm sac, expanding to a diameter which is larger than the diameter of the aneurysm neck; a (central) opening in the neck bridge through which the coils are inserted into the aneurysm sac; and a closure mechanism which closes the opening after the coils have been inserted into the aneurysm sac.

In an example, the toroidal mesh can be the outer surface of a torus. This is analogous to the outer surface of a bagel. Following this analogy, the central opening in the toroidal mesh is analogous to the hole in a bagel, although probably not as relatively large as the hole in a bagel. In an example, the cross-sectional area of the central opening in the toroidal mesh can be between 5% to 15% of the maximum cross-sectional area of the toroidal mesh. In an example, the cross-sectional area of the central opening in the toroidal mesh can be between 10% to 30% of the maximum cross-sectional area of the toroidal mesh. In an example, the central opening can have a hyperbolic cross-section. In an example, a toroidal mesh can radially expand within the aneurysm sac to a width which is greater than the width of the aneurysm neck. In an example, a toroidal mesh can be created geometrically (e.g. modeled) by rotating a circle or ellipse around a vertical axis (in three-dimensional space) which is to the right of the circle or ellipse.

In an example, a toroidal mesh (e.g. neck bridge) can have uniform porosity. In an example, a toroidal mesh can have a uniform durometer level. In an example, a toroidal mesh can have uniform elasticity. In an example, the outer perimeter of the toroidal mesh can have greater porosity than the central portion of the toroidal mesh. In an example, the outer perimeter of the toroidal mesh can have a greater durometer level than the central portion of the toroidal mesh. In an example, the outer perimeter of the toroidal mesh can be more elastic than the central portion of the toroidal mesh. In an example, the outer perimeter of the toroidal mesh can have lower porosity than the central portion of the toroidal mesh. In an example, the outer perimeter of the toroidal mesh can have a lower durometer level than the central portion of the toroidal mesh. In an example, the outer perimeter of the toroidal mesh can be less elastic than the central portion of the toroidal mesh.

In an example, a valve in a central opening of a torus-shaped neck bridge can be a leaflet valve. In an example, a valve in a central opening can be a bi-leaflet valve or tri-leaflet valve, analogous to a heart valve. In an example, a valve can passively open when an embolic coil is pushed through it and passively close when the end of the coil passes or when a portion of the coil is detached and removed. In an example, such a valve allows embolic coils to be inserted into the aneurysm after the toroidal mesh has been expanded in the aneurysm, but closes to reduce blood flow into the aneurysm after the end of the coil has passed through the valve.

In an alternative example, an active valve can be remotely opened and/or closed by the operator of the device. In an example, an active valve can be remotely opened and/or closed by an operator by the application of electromagnetic energy. In an example, an active valve can be remotely opened and/or closed by an operator by pulling a filament. In an example, an active valve can be remotely opened and/or closed by an operator by pushing, pulling, or rotating a wire. In an example, an active valve can be remotely opened and/or closed by an operator by cutting, pulling, or pushing a flap or plug. Relevant variations discussed elsewhere in this disclosure or in priority-linked disclosures can also be applied to this example.

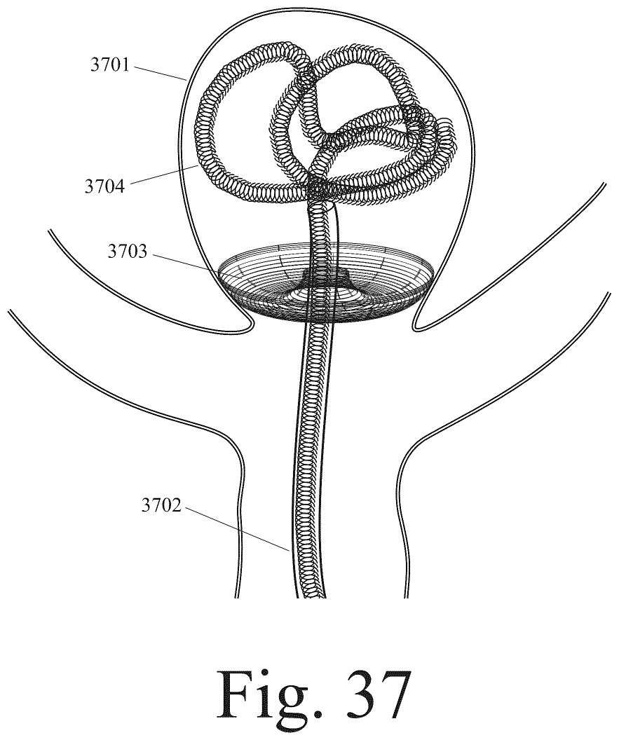

FIG. 37 shows an intrasaccular device for occluding a cerebral aneurysm comprising: a catheter 3702; a half-torus mesh (e.g. neck bridge) 3703 which is configured to be radially expanded within an aneurysm 3701 to bridge the neck of the aneurysm, wherein there is a central opening in the half-torus mesh; and one or more embolic coils (or other embolic members) 3704 which are inserted through the central opening into the aneurysm. In an example, this device can further comprise a valve in the central opening through which the one or more embolic coils are inserted.

In an example, a half-torus mesh can be the lower surface of the lower half of a torus. This is analogous to the lower surface of a half of a bagel lying flat on a surface. Following this analogy, the central opening in the half-torus is analogous to the hole in a half bagel, although probably not as relatively large as the hole in a half bagel. In an example, the cross-sectional area of the central opening in the half-torus mesh can be between 5% to 15% of the maximum cross-sectional area of the half-torus mesh. In an example, the cross-sectional area of the central opening in the half-torus mesh can be between 10% to 30% of the maximum cross-sectional area of the half-torus mesh.

In an example, a half-torus mesh can be created geometrically (e.g. modeled) by rotating an upward-opening arc (e.g. a section of a circle) around a vertical axis (in three-dimensional space) which is to the right of the arc. In an example, the central portion of a half-torus mesh can comprise an upward-rising cone, analogous to the cone of a volcano, with the opening being where the crater of a volcano would be. In an example, the half-torus mesh can radially expand within the aneurysm sac to a width which is greater than the width of the aneurysm neck.

In an example, a half-torus mesh can have uniform porosity. In an example, a half-torus mesh can have a uniform durometer level. In an example, a half-torus mesh can have uniform elasticity. In an example, the outer perimeter of the half-torus mesh can have greater porosity than the central portion of the half-torus mesh. In an example, the outer perimeter of the half-torus mesh can have a greater durometer level than the central portion of the half-torus mesh. In an example, the outer perimeter of the half-torus mesh can be more elastic than the central portion of the half-torus mesh. In an example, the outer perimeter of the half-torus mesh can have lower porosity than the central portion of the half-torus mesh. In an example, the outer perimeter of the half-torus mesh can have a lower durometer level than the central portion of the half-torus mesh. In an example, the outer perimeter of the half-torus mesh can be less elastic than the central portion of the half-torus mesh.

In an example, a valve in a central opening of a half-torus neck bridge can be a leaflet valve. In an example, a valve in a central opening can be a bi-leaflet valve or tri-leaflet valve, analogous to a heart valve. In an example, a valve can passively open when an embolic coil is pushed through it and passively close when the end of the coil passes or when a portion of the coil is detached and removed. In an example, such a valve allows embolic coils to be inserted into the aneurysm after the half-torus mesh has been expanded in the aneurysm, but closes to reduce blood flow into the aneurysm after the end of the coil has passed through the valve.

In an alternative example, an active valve can be remotely opened and/or closed by the operator of the device. In an example, an active valve can be remotely opened and/or closed by an operator by the application of electromagnetic energy. In an example, an active valve can be remotely opened and/or closed by an operator by pulling a filament. In an example, an active valve can be remotely opened and/or closed by an operator by pushing, pulling, or rotating a wire. In an example, an active valve can be remotely opened and/or closed by an operator by cutting, pulling, or pushing a flap or plug. Relevant variations discussed elsewhere in this disclosure or in priority-linked disclosures can also be applied to this example.

I claim:

1. An intrasaccular aneurysm occlusion device comprising:
a torus-shaped neck bridge which is configured to be inserted into an aneurysm sac and then expanded to cover the neck of the aneurysm, wherein the shape of the neck bridge can be modeled by revolving a convex shape in three-dimensional space around an axis of revolution which is coplanar with the convex shape, wherein a convex shape diameter is the widest diameter of the convex shape which is perpendicular to the axis of revolution, wherein a revolution diameter is the widest diameter of the circle formed by the path of the center of the convex shape as the convex shape is revolved in three-dimensional space, and wherein the revolution diameter is greater than the convex shape diameter.

2. The device in claim 1 wherein the device further comprises one or more embolic members or embolic material which is inserted into the aneurysm sac through a central opening in the neck bridge.

3. The device in claim 1 wherein the revolution diameter is 5% to 30% greater than the convex shape diameter.

4. The device in claim 1 wherein the convex shape is a circle.

5. The device in claim 1 wherein the convex shape is an ellipse with a longitudinal axis which is perpendicular to the axis of revolution.

6. The device in claim 1 wherein the convex shape is an ellipse with a longitudinal axis which is parallel to the axis of revolution.

7. The device in claim 1 wherein the convex shape is an ellipse wherein an extension of a longitudinal axis of the ellipse intersects the axis of revolution at an acute angle.

8. The device in claim 1 wherein the convex shape is the perimeter of the yin portion or the yang portion of a yin-yang symbol.

9. The device in claim 1 wherein the convex shape is the perimeter of a teardrop shape.

10. An intrasaccular aneurysm occlusion device comprising:
a torus-section-shaped neck bridge which is configured to be inserted into an aneurysm sac and then expanded to cover the neck of the aneurysm, wherein the shape of the neck bridge can be modeled by revolving a section of a convex shape in three-dimensional space around an axis of revolution which is coplanar with the convex shape, wherein a convex shape diameter is the widest diameter of the convex shape which is perpendicular to the axis of revolution, wherein a revolution diameter is the widest diameter of the circle formed by the path of the center of the convex shape as the convex shape is revolved in three-dimensional space, and wherein the revolution diameter is greater than the convex shape diameter.

11. The device in claim 10 wherein the device further comprises one or more embolic members or embolic material which is inserted into the aneurysm sac through a central opening in the neck bridge.

12. The device in claim 10 wherein the revolution diameter is 5% to 30% greater than the convex shape diameter.

13. The device in claim 10 wherein the section of a convex shape is a semi-circle.

14. The device in claim 10 wherein the section of a convex shape is the perimeter of a circle with a gap within a portion between 9 o'clock and 3 o'clock coordinates.

15. The device in claim 10 wherein the section of a convex shape is the perimeter of a circle with a gap within a portion between 1 o'clock and 5 o'clock coordinates.

16. The device in claim 10 wherein the section of a convex shape is a section of an ellipse with a longitudinal axis which is perpendicular to the axis of revolution.

17. The device in claim 10 wherein the section of a convex shape is a section of an ellipse with a longitudinal axis which is parallel to the axis of revolution.

18. The device in claim 10 wherein the section of a convex shape is a section of an ellipse wherein an extension of a longitudinal axis of the ellipse intersects the axis of revolution at an acute angle.

19. The device in claim 10 wherein the section of the convex shape is the perimeter of an ellipse with a gap within a portion between 1 o'clock and 5 o'clock coordinates.

20. An intrasaccular aneurysm occlusion device comprising:
a longitudinal catheter that is configured to be inserted into a blood vessel, wherein this blood vessel is the parent vessel from which an aneurysm has formed;
a longitudinal flexible embolic member that is configured to travel through the longitudinal lumen and be inserted into the aneurysm sac; and
an expandable toroidal member that is configured to travel through the longitudinal lumen, be inserted into the aneurysm sac, and be expanded within the aneurysm sac; wherein this toroidal member is configured to substantially occlude the aneurysm neck after it is expanded; wherein the longitudinal flexible embolic member is inserted into the aneurysm sac through a central opening in the expandable toroidal member; and wherein the expandable toroidal member prevents the longitudinal flexible member from protruding into the parent vessel of the aneurysm.
